SEQUENCE LISTING (4 sequences)

SEQ ID NO: 1 - DNA, 64 bp, Artificial Sequence
  Description of Artificial Sequence Synthetic oligonucleotide
tgctgaagaactggctaatgaacggtgttttggccactgactgacaccgttcaagccagttctt

SEQ ID NO: 2 - DNA, 64 bp, Artificial Sequence
  Description of Artificial Sequence Synthetic oligonucleotide
cctgaagaactggcttgaacggtgtcagtcagtggccaaaacaccgttcattagccagttcttc

SEQ ID NO: 3 - DNA, 65 bp, Artificial Sequence
  Description of Artificial Sequence Synthetic oligonucleotide
tgctggaaatgtactgcgcgtggagacgttttggccactgactgacgtctccacgcagtacattt

SEQ ID NO: 4 - DNA, 65 bp, Artificial Sequence
  Description of Artificial Sequence Synthetic oligonucleotide
cctggaaatgtactgcgcgtggagacgttttggccactgactgacgtctccacgcagtacatttc

(19) United States
(12) Patent Application Publication
(10) Pub. No.: US 2013/0023491 A1
(43) Pub. Date: Jan. 24, 2013

(54) BETA-CELL REPLICATION PROMOTING COMPOUNDS AND METHODS OF THEIR USE

(71) Applicants: Justin P. Annes, Brookline, MA (US); Douglas A. Melton, Lexington, MA (US); Lee L. Rubin, Wellesley, MA (US); Gordon Weir, Cambridge, MA (US)

(72) Inventors: Justin P. Annes, Brookline, MA (US); Douglas A. Melton, Lexington, MA (US); Lee L. Rubin, Wellesley, MA (US); Gordon Weir, Cambridge, MA (US)

(73) Assignees: President and Fellows of Harvard College, Cambridge, MA (US); Joslin Diabetes Center, Inc., Boston, MA (US); The Brigham and Women's Hospital, Inc., Boston, MA (US)

(21) Appl. No.: 13/516,278

(22) PCT Filed: Dec. 17, 2010
(86) PCT No.: PCT/US2010/061075

§ 371 Date: Sep. 17, 2012

Related U.S. Application Data
(60) Provisional application No. 61/288,001, filed on Dec. 18, 2009.

(51) Int. Cl. A61K 31/7064; A61K 31/5377; A61P 3/10; A61K 31/52; C40B 30/06; C12N 9/12; C12N 9/99; A61K 31/7056

(52) U.S. Cl. 514/49; 435/184; 514/234.2; 514/52; 514/263.4; 506/10; 435/194

(57) ABSTRACT

In the invention provides for a method of stimulating or increasing β-cell replication or growth, by contacting a β-cell with an inhibitor of adenosine kinase (ADK), an inhibitor of S-Adenosylhomocysteine hydrolase (SAHH) or an activator of AMP activated protein kinase (AMPK).

### CROSS REFERENCE TO RELATED APPLICATIONS

This application claims benefit under 35 U.S.C. §119(e) of the U.S. Provisional Application No. 61/288,001 filed Dec. 18, 2009, the content of which is incorporated herein by reference in its entirety.

### GOVERNMENT SUPPORT

This invention was made with government support under grant no. DK072505 and DK084206 awarded by the National Institutes of Health. The government has certain rights in the invention.

### FIELD OF THE INVENTION

The invention relates to compositions and methods of promoting β-cell replication and/or growth.

### BACKGROUND OF THE INVENTION

There are two forms of diabetes mellitus: (1) insulin dependent or Type 1 diabetes (a.k.a., Juvenile Diabetes, Brittle Diabetes, Insulin Dependent Diabetes Mellitus (IDDM)) and (2) non-insulin-dependent or Type II diabetes (a.k.a., NIDDM). Type 1 diabetes develops most often in young people but can appear in adults. Type 2 diabetes develops most often in middle aged and older adults, but can appear in young people. Diabetes is a disease derived from multiple causative factors and characterized by elevated levels of plasma glucose (hyperglycemia) in the fasting state or after administration of glucose during an oral glucose tolerance test. A decrease in β-cell mass occurs in both Type I and Type II diabetes.

Type 1 diabetes is an autoimmune disease condition characterized by high blood glucose levels caused by a total lack of insulin, i.e., a loss of pancreatic β-cell function and mass. Type 1 diabetes occurs when a person's immune system attacks the insulin producing β-cells in the pancreas and destroys them. It is believed that the Interleukin 12 (IL-12) family of cytokines and downstream activation of Signal Transducers and Activators of Transcription (STAT) family members, e.g., STAT-4, which are believed to be regulators of T cell differentiation involved in immune responses, play a major role in the processes that lead to autoimmune β-cell destruction. The pancreas then produces little or no insulin. The most common Type 1 diabetes symptoms experienced include excessive thirst (polydipsia), frequent urination (polyuria), extreme hunger (polyphagia), extreme fatigue, and weight loss. These symptoms are caused by hyperglycemia and a breakdown of body fats. Persons diagnosed with Type 1 diabetes typically exhibit blood sugar levels over 300 mg and ketones present in their urine. Restoration of β-cell mass and insulin production can fully reverse the diabetic state. Evidence suggests that people with long standing Type 1 diabetes have β-cells that continue to form but are undesirably destroyed by continued autoimmune destruction. Therefore, compositions and methods for increasing β-cell replication would provide an effective way to restore normal β-cell mass levels and reverse or cure Type 1 diabetes.

LADA is a newly recognized subset of Type 1 diabetes and is thought to account for up to 10%-20% of all cases of diabetes. LADA is also known as Type 1.5 diabetes. LADA is often present in people initially diagnosed with Type 2 diabetes. Although it has characteristics similar to adult onset Type 1 diabetes, the β-cell destruction is considered to be less aggressive in its progression.

Type 2 diabetes results from a combination of insulin resistance and impaired insulin secretion but ultimately many people with Type 2 diabetes show markedly reduced pancreatic β-cell mass and function which, in turn, causes Type 2 diabetic persons to have a “relative” deficiency of insulin because pancreatic β-cells are producing some insulin, but the insulin is either too little or isn't working properly to adequately allow glucose into cells to produce energy. Recent autopsy studies have shown clear evidence of ongoing β-cell death (apoptosis) in people with Type 2 diabetes. Therefore, therapeutic approaches to provide more β-cells could provide a significant treatment for reversing or curing Type 2 diabetes.

Uncontrolled Type 2 diabetes leads to excess glucose in the blood, resulting in hyperglycemia, or high blood sugar. A person with Type 2 diabetes experiences fatigue, increased thirst, frequent urination, dry, itchy skin, blurred vision, slow healing cuts or sores, more infections than usual, numbness and tingling in feet. Without treatment, a person with Type 2 diabetes will become dehydrated and develop a dangerously low blood volume. If Type 2 diabetes remains uncontrolled for a long period of time, more serious symptoms may result, including severe hyperglycemia (blood sugar over 600 mg) lethargy, confusion, shock, and ultimately “hyperosmolar hyperglycemic non-ketotic coma” Persistent or uncontrolled hyperglycemia is associated with increased and premature morbidity and mortality. As such, therapeutic control of glucose homeostasis, lipid metabolism, obesity, and hypertension are critically important in the clinical management and treatment of diabetes mellitus.

The object of diabetes treatments is to prevent the occurrence of the above-mentioned chronic complications, slow disease progression by improving hyperglycemic status, or reversing/curing it. Conventional methods for treating diabetes have included administration of fluids and insulin in the case of Type 1 diabetes and administration of various hypoglycemic agents in the case of Type 2 diabetes. Hypoglycemic agents such as insulin preparations, insulin secretagogues, insulin sensitizers and a-glucosidase inhibitors have been widely applied as the method for the clinical treatment. Examples include acarbose (PrecoseJ), glimeprimide (AmarylJ), metformin (GlucophageV), nateglinide (StarlixV), pioglitazone (ActosV), repaglinide (PrandinJ), rosiglitazone (AvandiaV), sulfonylureas, Orlistat (XenicalV), exenatide (Byetta), and the like. Many of the known hypoglycemic agents, however, exhibit undesirable side effects and are toxic in certain cases. For example, in the case of the diabetic patients with seriously lowered pancreatic insulin secretion, effectiveness of insulin secretagogues and insulin sensitizers is diminished. Similarly, in the case of the diabetic patients whose insulin resistance is significantly high, effectiveness of insulin preparations and insulin secretagogues is diminished.

In principle, diabetes mellitus could be treated by a successful transplant of the tissue containing cells that secrete or produce insulin, i.e., the islets of Langerhans. Transplantation of insulin producing cells has been tried as a method to reverse or cure Type 1 diabetes, but there are significant risks associated with the surgery and with the toxic immunosuppression type drugs that need to be taken to prevent or mitigate allograft rejection and autoimmune reoccurrence. In addition, there are over 1 million people with Type 1 diabetes in the United States today, but the supply of cadaveric pancreatic tissue for islets is limited. For instance, only 6,000 organs are available per year and 2 or 3 organs are needed to provide enough islets to reverse Type 1 diabetes in one person. Therefore, providing a new source of functioning (insulin producing) β-cells is urgently needed.

Only one example of a high-throughput screening assay for compounds that can induce β-cell proliferation is described in the art. The screening method utilizes growth-arrested, reversibly immortalized mouse β-cells. The major drawback with this approach is the compounds identified in this screening assay may not have the same effect on primary β-cells, e.g., identified compounds are specific for inducing proliferation in growth-arrested, reversibly immortalized mouse β-cells only. Thus, there is a need for methods for screening compounds that can induce proliferation of primary β-cell, e.g., β-cells that have not undergone transformation.

### SUMMARY OF THE INVENTION

In one aspect the invention provides for a method of increasing β-cell replication in a population of pancreatic cells, the method comprising: contacting a population of pancreatic cells with an inhibitor of adenosine kinase (ADK), an inhibitor of S-Adenosylhomocysteine Hydrolase (SAHH), or an activator of AMP activated protein kinase (AMPK).

In another aspect, the invention provides for a method of screening for a candidate compound for increasing β-cell replication, the method comprising: (a) contacting a population of pancreatic cells with a test compound; (b) selecting the compound that: (i) increases the total number of cells in the culture, (ii) increases the total number of cells expressing at least one β-cell marker in the culture, as compared to an untreated control, (iii) increases the ratio of cells expressing at least one β-cell marker to the total number of cells in the culture, as compared to an untreated control, (iv) increases the number of cells expressing at least one cell-replication marker, as compared to an untreated control, or (iv) increases the ratio of cells expressing at least one cell-replication marker, as compared to an untreated control; and wherein pancreatic cells are primary pancreatic cells.

### BRIEF DESCRIPTION OF THE DRAWINGS

FIG. 1 depicts a bar graph showing % increase in PH3 over PDX1 by A10 and B8.

FIGS. 2a and 2b depict a line graphs showing the effect of compound A10 (5-IT) concentration on β-cell (FIG. 2a) and mouse dermal fibroblast (FIG. 2b) proliferation. EC50 for β-cell was determined to be 0.47 μM.

FIG. 3 depicts a line graph showing the effect of compound B8 concentration on β-cell proliferation (EC50=9.1 μM).

FIG. 4 depicts a line graph showing the effect of compounds A10 and B8 on Ki-67 fold induction over a period of 4 days after treatment. Compounds were added to the cells on day 0 and media was changed on day 2.

FIG. 5 depicts a bar graph showing the effect of media serum concentration on Ki-67 induction in β-cells by 1 μM of A10.

FIG. 6 depicts a line graph showing that glucose does not effect induction of Ki-67 and PDX+ expression by A10 in β-cells.

FIG. 7 depicts a line graph showing that adenosine does not lead to induction of PDX+ and Ki-67 in Rat islets in a concentration dependent manner.

FIGS. 8a and 8b depict bar graphs showing the effect of adenosine receptor antagonists on A10 induced increase in Ki-67. FIG. 9a, antagonists were tested at concentrations of 2 μM, 1 μM, 0.2 μM, 0.04 μM, or 0.0 μM (no antagonist). FIG. 9b, antagonists tested at concentration of 0.2 μM.

FIG. 9 depicts a bar graph showing the effect of adenosine kinase inhibitor A10 and adenosine monophosphate activate kinase activator AICAR on β-cell replication.

FIG. 10 depicts a bar graph showing the effect of AMPK-inhibition on A10 induced β-cell replication.

FIG. 11a and 11b depict structures of some exemplary nucleoside based (FIG. 11a) and non-nucleoside based (FIG. 11b) adenosine kinase inhibitors activators.

FIG. 12 is a bar graph showing increase in beta-cell replication in vivo.

FIG. 13a-13d show that adenosine kinase inhibitors (ADK-Is) induce proliferation of rat, mouse and porcine β-cells. FIG. 13a show the chemical structures and names of some exemplary ADK-Is that promote β-cell replication. FIG. 13b shows the dose-response curves showing the relationship between murine (top) and porcine (bottom) β-cell proliferation in response to ABT-702 treatment. FIG. 13c shows the dose-response curves for rat islet cultures showing the relationship between β-cell proliferation and compound treatment with 5-IT (left; EC50=4.7 μM) and ABT-702 (right; EC50=7.0 μM). FIG. 13d shows the quantitation of β-cell number after treatment with DMSO or 5-IT (2 μM) for 96 h and 144 h. Error bars represent standard deviation, *P<0.01 compared to the vehicle treatment condition.

FIGS. 14a and 14b show that β-cells express nuclear adenosine kinase (ADK) which acts as a cell-autonomous negative regulator of proliferation. FIG. 14a is a western blot showing siRNA-mediated ADK knockdown evaluated using H4IIE (rat hepatocyte cell-line) lysate from cells stably transduced with either a negative control siRNA (lanes 1,3) or an ADK-directed siRNA (lanes 2,4). Loading was standardized using γ-tubulin. FIG. 14b is a bar graph showing quantitation of β-cell replication after infection with virus containing either a control siRNA sequence (left) or an ADK-directed siRNA sequence (right). The replication rate of uninfected cells and infected cells within the same well were analyzed separately on the basis of virus-encoded GFP expression. Error bars represent SEM (n=8 independent wells); *P<0.01.

FIGS. 15a and 15b show that induction of β-cell replication by ADK-Is is additive to glucose and glucagon-like peptide 1 receptor (GLP-1R) agonists. FIG. 15a is a bar graph showing the quantitation of the β-cell replication rate after culture for 24 h, 48 h or 96 h in the presence of various glucose concentrations plus DMSO or 5-IT (2 μM). Values are normalized to the 5 mM glucose plus DMSO treatment condition at each time point. The standard deviation was less than 10% for each treatment condition, error bars not shown.*P<0.01 when 5-IT treated wells are compared to DMSO treated wells at the same glucose concentration and time point; **P<0.01 when DMSO or 5-IT treated wells are compared to the 5 mM glucose treated condition at the same time point and with same treatment (DMSO or 5-IT). FIG. 15b is a bar graph showing the quantitation of the β-cell replication rate after treatment with DMSO, 5-IT, GLP-1, Ex4, 5-IT plus GLP-1 or 5-IT plus Ex4 for 24 h. The concentration of 5-IT was 2 μM. The concentrations of GLP-1 and Ex-4 were 20 nM (left bar) and 4 nM (right bar). Values normalized to the DMSO treatment condition. Error bars represent standard deviation; *P<0.01 and **P<0.03 for the indicated comparisons.

FIGS. 16a-16e show that ADK-Is selectively promote β-cell replication in vitro and in vivo. FIG. 18a shows bar graphs depicting quantitation of the in vitro replication rates of δ-cells (somatostatin⁺; left panel), α-cells (glucagon⁺; center panel) and fibroblasts (vimentin⁺; right panel) after treatment with DMSO, 5-IT (2 μM) or ABT-702 (15 μM). *P<0.01 and **P<0.05 compared to DMSO treated condition. FIG. 16b is a bar graph showing quantitation of the replication rate of isolated murine hepatocytes grown in the presence of EGF (40 ng/ml) and HGF (20 ng/ml) plus DMSO or ABT-702 (15 μM). FIG. 16c is a bar graph showing quantitation of in vivo replication rates of islet β-cells in mice 24 h after treatment with BRDU and either vehicle or ABT-702. FIG. 16d is a bar graph showing quantitation of in vivo replication rates of exocrine cells in mice 24 h after treatment with BRDU and either vehicle or ABT-702. FIG. 16e is a bar graph showing quantitation of in vivo replication rates of hepatocytes in mice 24 h after treatment with BRDU and either vehicle or ABT-702. Error bars represent the standard deviation, p-values obtained using two-tailed t test.

FIGS. 17a and 17b are bar graphs showing β-cell replication is increased by ADK-Is. FIG. 17a shows quantitation of β-cell replication after treatment with DMSO or 5-IT (2 μM). β-cells were identified by the presence of PDX-1 and insulin staining. *P<0.001. FIG. 17b shows quantitation of β-cell replication after treatment with DMSO, 7-Iodo-2,3-dideoxy-7-deazadenosine (20 μM, 10 μM) or Aristeromycin (6 μM, 1 μM). Error bars represent standard deviation.

FIG. 18a is a bar graph showing quantitation of β-cell replication using the co-expression of PDX-1 and phosphohiston-H3. Cultures were treated with DMSO, 5-IT (2 μM) or ABT-702 (15 μM).). *P<0.001 compared to DMSO treated condition.

FIG. 18b is a bar graph showing quantitation of β-cell replication using a two day BRDU pulse in the presence of DMSO, 5-IT (2 μM) or ABT-702 (15 μM) followed by a two day chase without compound treatment. The data is displayed as the fold-increase in BRDU⁺ PDX-1⁺ cells in compound treated wells compared to DMSO-treated wells. *P<0.002 compared to DMSO.

FIG. 19 is a bar graph showing automated quantitation of the percentage of ADK-positive islet cells after infection with a control siRNA or an ADK-directed siRNA. Error bars represent standard deviation; *P<0.001.

FIG. 20 is a bar graph showing in vivo treatment of mice with ABT-702 increases β-cell replication but not exocrine cells. Quantitation of the in vivo β-cell replication rate using insulin immunostaining to identify β-cells and BRDU immunostaining to identify replicating cells (left). Quantitation of the extra-islet exocrine cell replication rate was simultaneously performed using the same tissue sections (right).

FIG. 21 depicts a schematic outline of the screening protocol used to identify compounds that promote β-cell replication.

FIG. 22 depicts a bar graph showing the effect of SAHH inhibitor vidarabine on β-cell proliferation.

FIG. 23 is a schematic depicting an overview of the ADK pathway.

### DETAILED DESCRIPTION OF THE INVENTION

In one aspect the invention provides for a method of increasing β-cell replication in a population of pancreatic cells, the method comprising: contacting a population, or preparation, of pancreatic cells with an inhibitor of adenosine kinase (ADK), an inhibitor of S-Adenosylhomocysteine Hydrolase (SAHH), or an activator of AMP activated protein kinase (AMPK).

As used herein, “increasing β-cell replication” means that β-cells replicate at a faster rate and/or more frequently. In some embodiments of this and other aspects of the invention, β-cell replication is increased by at least 5%, 10%, 20%, 30%, 40%, 50%, 50%, 70%, 80%, 90%, 1-fold, 1.1-fold, 1.5-fold, 2-fold, 3-fold, 4-fold, 5-fold, 10-fold, 50-fold, 100-fold or more higher relative to an untreated control. The % or fold increase in β-cell replication can be determined by measuring number of replicating β-cells while in contact with a compound described herein relative to a control where the β-cells are not in contact with the compound. Increase in replication can also be based on ratios of replicating cells to total number of cells in the respective treated and untreated control. In some embodiments, total number of cells in the treated and untreated controls are used to determine the replication frequency.

In some embodiments, “increasing β-cell replication” also includes an increase in β-cell number due to differentiation of β-cell progenitors into β-cells. In an alternative embodiment, “increasing β-cell replication” does not include an increase in β-cell number due to differentiation of β-cell progenitors into β-cells.

As used herein, the term “β-cell” includes primary pancreatic β-cells, pancreatic β-like cells derived from dedifferentiated cells, e.g. from induced pluripotent stem cells (iPSCs), or pancreatic β-like cells that have been directly reprogrammed from a cell of endodermal origin (e.g. a liver cell or an exocrine pancreatic cell). In one embodiment, a β-cell is not an immortalized cell line (e.g. proliferate indefinitely in culture). In one embodiment, the β-cell is not a transformed cell, e.g, a cell that exhibits a transformation property, such as growth in soft agar, or absence of contact inhibition.

The term “pancreatic β-like cell,” as used herein, refers to a cell which expresses at least 15% of the amount of insulin expressed by an endogenous pancreatic beta-cell, or at least about 20%, or at least about 30%, or at least about 40%, or at least about 50%, or at least about 60%, or at least about 70%, or at least about 80%, or at least about 90%, or at least about 100% or greater than 100%, such as at least about 1.5-fold, or at least about 2-fold, or at least about 2.5-fold, or at least about 3-fold, or at least about 4-fold or at least about 5-fold or more than about 5-fold the amount of the insulin secreted by an endogenous pancreatic beta-cell. In one embodiment, the pancreatic β-like cell exhibits at least one, or at least two characteristics of an endogenous pancreatic beta-cell, for example, but not limited to, secretion of insulin in response to glucose, and expression of beta-cell markers, such as for example, c-peptide, Pdx-1 and glut-2. The pancreatic β-like cell is sometimes referred herein to as a “reprogrammed β-cell”, which are used interchangeably herein with the term “pancreatic β-like cell”. In one embodiment, the pancreatic β-like cell is not an immortalized cell (e.g. proliferate indefinitely in culture). In one embodiment, the pancreatic β-like cell is not a transformed cell, e.g, a cell that exhibits a transformation property, such as growth in soft agar, or absence of contact inhibition.

As used herein, the term “de-differentiated cell” refers to a cell that has been reprogrammed from a differentiated cell. The term “reprogrammed” or “reprogramming” as used herein refers to the process that alters or reverses the differentiation state of a somatic cell. The cell can either be partially or terminally differentiated prior to the reprogramming. Reprogramming encompasses complete reversion of the differentiation state of a somatic cell to a pluripotent cell. Such complete reversal of differentiation produces an induced pluripotent (iPS) cell. Reprogramming also encompasses partial reversion of the differentiation state, for example to a multipotent state or to a somatic cell that is neither pluripotent or multipotent, but is a cell that has lost one or more specific characteristics of the differentiated cell from which it arises, e.g. direct reprogramming of a differentiated cell to a different somatic cell type. Reprogramming generally involves alteration, e.g., reversal, of at least some of the heritable patterns of nucleic acid modification (e.g., methylation), chromatin condensation, epigenetic changes, genomic imprinting, etc., that occur during cellular differentiation as a zygote develops into an adult.

The methods described herein, are applicable to pancreatic β-like cells that have been derived from reprogrammed (de-differentiated) cells. For example, obtained from an iPS cell that has been differentiated into a pancreatic beta-like cell using factors and conditions known to those of skill in the art. The pancreatic β-like cells can also be derived by direct reprogramming of endoderm/exocrine somatic cells without reversion to the pluripotent stem cell state (e.g. iPS cell), for example as described in Zhou, et al. Nature, Vol 455, Oct. 2, 2008, pages 627-633), herein incorporated by reference in its entirety.

As used herein, the terms “iPS cell” and “induced pluripotent stem cell” are used interchangeably and refers to a pluripotent stem cell artificially derived, i.e. dedifferentiated (reprogrammed) from a non-pluripotent cell, typically an adult somatic cell, for example, by inducing a forced expression of one or more genes.

As used herein, the term “endogenous pancreatic beta-cell”, alternatively a “primary pancreatic beta-cell” refers to an insulin producing cell of the pancreas of a mammal, or a cell of a pancreatic beta-cell (beta cell) phenotype of a mammal. The phenotype of a pancreatic beta-cell is well known by persons of ordinary skill in the art, and include, for example, secretion of insulin in response to an increase in glucose level, expression of markers such as c-peptide, PDX-1 polypeptide and Glut 2, as well as distinct morphological characteristics such as organized in islets in pancreas in vivo, and typically have small spindle like cells of about 9-15 μm diameter. Endogenous pancreatic beta-cells can be found in the islets of Langerhans. In methods of the invention, the primary pancreatic beta-cells can be contacted in vitro as part of the islets of Langerhans.

As used herein, the term “insulin producing cell” includes primary beta-cells as that term is described herein, as well as pancreatic beta-like cells as that term is described herein, that synthesize (i.e., transcribe the insulin gene, translate the proinsulin mRNA, and modify the proinsulin mRNA into the insulin protein), express (i.e., manifest the phenotypic trait carried by the insulin gene), or secrete (release insulin into the extracellular space) insulin in a constitutive or inducible manner.

The term “a cell of endoderm origin” as used herein refers to a cell of endoderm origin includes any cell which has developed from an endoderm cell, which is a cell from one of the three primary gem layers in the very early embryo that differentiates to give rise to the embryonic gut then to the linings of the respiratory and digestive tracts and to the liver and pancreas. Studies in diverse model organisms and humans have revealed evolutionarily conserved inductive signals and transcription factor networks that elicit the differentiation of liver and pancreatic cells and provide guidance for how to promote hepatocyte and β cell differentiation from diverse stem and progenitor cell types.

In some embodiments of this and other aspects of the invention, activity of the adenosine kinase is inhibited or lowered by at least 5%, at least 10%, at least 20%, at least 30%, at least 40%, at least 50%, at least 60%, at least 70%, at least 80%, at least 90%, at least 95%, at least 98%, or 100% (e.g. complete loss of activity) relative to an uninhibited control. Without wishing to be bound by theory, activity of the adenosine kinase can be measured by measuring the phosphorylation of adenosine by utilizing methods known in the art for measuring such phosphorylation reactions.

In some embodiments of this and other aspects of the invention, the adenosine kinase inhibitor has an IC50 of less than or equal to 500 nM, less than or equal to 250 nM, less than or equal to 100 nM, less than or equal to 50 nM, less than or equal to 10 nM, less than or equal to 1 nM, less than or equal to 0.1 nM, less than or equal to 0.01 nM, or less than or equal to 0.001 nM.

In some embodiments of this and other aspects of the invention, activity of the AMP-activated kinase is increased by at least 5%, at least 10%, at least 20%, at least 30%, at least 40%, at least 50%, at least 60%, at least 70%, at least 80%, at least 90%, at least 1-fold, at least 1.1-fold, at least 1.5-fold, at least 2-fold, at least 3-fold, at least 4-fold, at least 5-fold, or more relative to an unactivated control.

In some embodiments of this and other aspects of the invention, activator of AMP-activated kinase has an EC50 of less than or equal to 500 nM, less than or equal to 250 nM, less than or equal to 100 nM, less than or equal to 50 nM, less than or equal to 10 nM, less than or equal to 1 nM, less than or equal to 0.1 nM, less than or equal to 0.01 nM, or less than or equal to 0.001 nM.

In some embodiments of this and other aspects of the invention, activity of the SAHH is inhibited or lowered by at least 5%, at least 10%, at least 20%, at least 30%, at least 40%, at least 50%, at least 60%, at least 70%, at least 80%, at least 90%, at least 95%, at least 98%, or 100% (e.g. complete loss of activity) relative to an uninhibited control. Activity of the SAHH can be measured using the methods described, for example in, U.S. patent application Ser. No. 10/836,953 and 11/389,393, content of both of which is herein incorporated by reference.

In some embodiments of this and other aspects of the invention, the SAHH inhibitor has an IC50 of less than or equal to 500 nM, less than or equal to 250 nM, less than or equal to 100 nM, less than or equal to 50 nM, less than or equal to 10 nM, less than or equal to 1 nM, less than or equal to 0.1 nM, less than or equal to 0.01 nM, or less than or equal to 0.001 nM.

It will be recognized that many compounds described herein can exert multiple effects on a cell. For example, SAH analogues and tubercidin act at various points in methylation pathways. Thus, division of the compounds and molecules into certain groups, e.g. ADK inhibitors or SAHH inhibitors, is not intended to be an exact and non-overlapping scientific division. Rather such information is set forth to assist those in the art with understanding certain scientific data that were considered by the inventors. For example, many of the ADK inhibitors described herein are also amenable to the invention as SAHH inhibitors.

In some embodiments of this and other aspects of the invention, the adenosine kinase inhibitor is of formula (I):

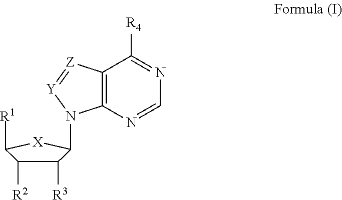

wherein:

R¹ is H, optionally substituted C₁-C₆ alkyl, optionally substituted C₂-C₆ alkenyl, optionally substituted C₂-C₆ alkynyl, OR⁵, SR⁵, N(R⁶)₂, (CH₂)ₘR⁷, or R¹ and R² together with the atoms they are attached to form 5-8 membered heterocycle which can be optionally substituted;

R² and R³ are each independently H, OR⁵, SR⁵, N(R⁵)₂, or R² and R³ together with the atoms they are attached to form 5-8 membered heterocyclyl which can be optionally substituted;

R⁴ is H, halogen, CN, N₂, OR⁵, SR⁵, N(R⁵)₂, optionally substituted C₁-C₆ optionally substituted C₂-C₆ alkenyl, optionally substituted C₂-C₆ alkynyl, optionally substituted aryl, optionally substituted heteroaryl, optionally substituted cyclyl, optionally substituted heterocyclyl, optionally substituted arylalkyl, or optionally substituted heteroarylalkyl;

R⁵ is independently for each occurrence H, C(O)R⁷, C(O)OR⁷, C(O)N(R⁷)₂, optionally substituted C₁-C₆ alkyl, optionally substituted C₂-C₆ alkenyl, optionally substituted C₂-C₆ alkynyl, optionally substituted aryl, optionally substituted heteroaryl, optionally substituted cyclyl, optionally substituted heterocyclyl, optionally substituted arylalkyl, optionally substituted heteroarylalkyl, or the two R⁵ taken together with the nitrogen atom to which they are attached form a 5-to-7 membered ring optionally comprising 1-3-additional heteroatoms selected from N, O or S;

R⁶ is R⁵, OR⁵, SR⁵, N(R⁵)₂, N₂, CN, halogen, or

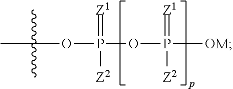

R⁷ is independently for each occurrence H, optionally substituted C₁-C₆ alkyl, optionally substituted C₂-C₆ alkenyl, optionally substituted C₂-C₆ alkynyl, optionally substituted aryl, optionally substituted heteroaryl, optionally substituted cyclyl, optionally substituted heterocyclyl, optionally substituted arylalkyl, or optionally substituted heteroarylalkyl;

X is O, S, NH, or CH₂;

Y and Z are each independently N or CR⁸;

R⁸ is independently for each occurrence H, halogen, CN, C(O)R⁷, C(O)OR⁷, C(O)N(R⁷)₂, optionally substituted C₁-C₆ alkyl, optionally substituted C₂-C₆ alkenyl, optionally substituted C₂-C₆ alkynyl, optionally substituted aryl, optionally substituted heteroaryl, optionally substituted cyclyl, optionally substituted heterocyclyl, optionally substituted arylalkyl, or optionally substituted heteroarylalkyl;

Z¹ is independently for each occurrence O or S;

Z² is independently for each occurrence OM, SM, OR⁵, SR⁵, N(R⁵)₂, optionally substituted C₁-C₆ alkyl, optionally substituted C₂-C₆ alkenyl, optionally substituted C₂-C₆ alkynyl, optionally substituted aryl, optionally substituted heteroaryl, optionally substituted cyclyl, optionally substituted heterocyclyl, optionally substituted arylalkyl, or optionally substituted heteroarylalkyl;

M is an alkali metal cation;

m is 1, 2, 3, or 4;

n is 0, 1, or 2; and

pharmaceutically acceptable salts and amides thereof.

In some preferred embodiments, M is Na⁺.

In some embodiments, R¹ is optionally substituted C₁-C₆ alkyl, OR⁵, N(R⁵)₂, or (CH₂)ₘR⁶. Preferably C₁-C₆ alkyl is methyl. When R¹ is N(R⁵)₂, at least one of R⁵ is H, preferably both R⁵ are H. When R¹ is OR⁵, R⁵ can be H or C₁-C₆ alkyl, preferably R⁵ is H.

When R¹ is (CH₂)ₘR⁶, m is 1 or 2, preferably m is 1. In some embodiments, R⁶ is OR⁵ or N(R⁵)₂. When R⁶ is N(R⁵)₂, at least one of R⁵ is H, preferably both R⁵ are H. When R⁶ is OR⁵, R⁵ can be H or optionally substituted C₁-C₆ alkyl, preferably R⁵ is H. Most preferably R¹ is CH₃, CH₂OH or NH₂.

In some embodiments, R¹ and R², in the compounds of formula (I), together with the atoms they are attached to form a 5-8 membered heterocycle, wherein the backbone of the heterocycle comprises

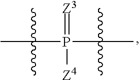

wherein Z³ is independently for each occurrence O or S and Z⁴ is H, OM, SM, OR⁵, SR⁵, N(R⁵)₂, optionally substituted C₁-C₆ alkyl, optionally substituted C₂-C₆ alkenyl, optionally substituted C₂-C₆ alkynyl, optionally substituted aryl, optionally substituted heteroaryl, optionally substituted cyclyl, optionally substituted heterocyclyl, optionally substituted arylalkyl, or optionally substituted heteroarylalkyl.

In some embodiments, at least one of Y or Z is CR⁸, preferably Y is CR⁸ and more preferably Y is CH₂. In one embodiment, Y is CR⁸ and Z is N.

In some other embodiments, both of Y and Z are CR⁸. Preferably Y is CH₂ and Z is CR⁸, wherein R⁸ is selected from the group consisting of CN, halogen, aryl, and heteroaryl. Preferably the aryl group is a phenyl group, which can be optionally substituted. When R⁸ is halogen, I and Br are preferred, with I being more preferred.

In still some other embodiments, R⁴ is a halogen or N(R⁵)₂. Halogens include, Br, F, I, or Cl, preferably halogen is Cl. When R⁴ is N(R⁵), both R⁵ can be H, or preferably one R⁵ is H and other R⁵ is selected from the group consisting of aryl and heteroaryl. Preferably the aryl group is a phenyl group. In some instances the phenyl group is an optionally substituted phenyl group, e.g., 4-fluoro-phenyl group.

In yet still some other embodiments, X is selected from the group consisting of O, NH and CH₂. In some preferred embodiments, X is O.

In some embodiments, R² and R³ are both OR⁵. Preferably R² and R³ are both OH.

In some embodiments, the compound of formula (I) has the stereochemical configuration shown in formula (Ia):

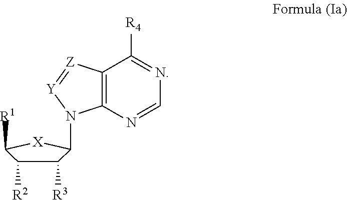

In other embodiments, the compound of formula (I) has the stereochemical configuration shown in formula (Ib):

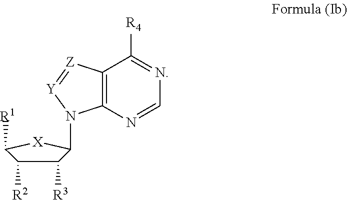

In some embodiments, the compound of formula (I) is not 7-deaza-7-iodo-2′-deoxyadenosine, 7-deaza-2′-deoxyadenosine, 7-deaza-2′,3′-dideoxyadenosine, Sangivamycin, Tubercidin, or adenosine.

In some embodiments, the compound of formula (I) is aristeromycin, 5′-deoxyadenosine, 5′-aminoadenosine, 5′-deoxy-5-iodotubercidin, 5-iodotubercidin (also referred to as A10 or 5-IT herein), 7-deaza-7-iodo-2′,3′-dideoxyadenosine (also referred to as dideoxy-7-iodo-deazaadenosie or d7IdAdo herein), nor-aristeromycin, nor-tubercidin, A-134974, Toyocamycin, GP-515 ((2R,3R,4S,5R)-2-(4-amino-3-bromo-1H-pyrazolo[3,4-d]pyrimidin-1-yl)-5-(aminomethyl)-tetrahydrofuran-3,4-diol), GP-3269 ((2R,3R,4S,5R)-2-(4-(4-fluorophenylamino)-5-phenyl-7H-pyrrolo[2,3-d]pyrimidin-7-yl)-tetrahydro-5-methylfuran-3,4-diol), GP-683 ((2R,3S,4R,5R)-tetrahydro-2-methyl-5-(5-phenyl-4-(phenylamino)-7H-pyrrolo[2,3-d]pyrimidin-7-yl)furan-3,4-diol), GP-947 ((2S,3S,4R,5R)-tetrahydro-2-methyl-5-(5-phenyl-4-(phenylamino)-7H-pyrrolo[2,3-d]pyrimidin-7-yl)furan-3,4-diol), compound 1 ((2R,3R,4S,5R)-2-(4-amino-5-iodo-7H-pyrrolo[2,3-d]pyrimidin-7-yl)-5-(aminomethyl)-tetrahydrofuran-3,4-diol), compound 2 ((2R,3R,4S,5R)-2-(4-chloro-5-iodo-7H-pyrrolo[2,3-d]pyrimidin-7-yl)-5-(aminomethyl)-tetrahydrofuran-3,4-diol), compound 3 ((1S,2R,3S,5R)-3-amino-5-(6-amino-9H-purin-9-yl)cyclopentane-1,2-diol), compound 4 ((1S,2R,3S,5R)-3-amino-5-(7-amino-3H-[1,2,3]triazolo[4,5-d]pyrimidin-3-yl)cyclopentane-1,2-diol), or compound 5 ((1S,2R,3S,4R)-4-(4-amino-5-iodo-7H-pyrrolo[2,3-d]pyrimidin-7-yl)cyclopentane-1,2,3-triol).

In some embodiments of this and other aspects of the invention, the adenosine kinase inhibitor is of formula (II):

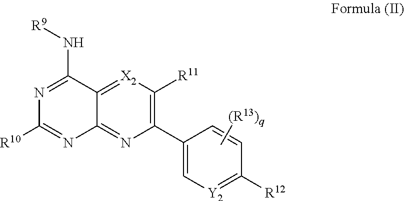

wherein:

each R⁹ is independently H, optionally substituted C₁-C₆ alkyl, optionally substituted C₂-C₆ alkenyl, optionally substituted C₂-C₆ alkynyl, optionally substituted aryl, optionally substituted heteroaryl, optionally substituted cyclyl, optionally substituted heterocyclyl, optionally substituted arylalkyl, optionally substituted heteroarylalkyl, or the two R⁹ taken together with the nitrogen atom to which they are attached form a 5-to-7 membered ring which optionally comprises 1-3-additional heteroatoms selected from N, O or S;

R¹⁰, R¹¹ and R¹² are each independently H, OR¹⁴, N(R¹⁴)₂, N₂, NO₂, CN, halogen, optionally substituted C₁-C₆ alkyl, optionally substituted C₂-C₆ alkenyl, optionally substituted C₂-C₆ alkynyl, optionally substituted aryl, optionally substituted heteroaryl, optionally substituted cyclyl, optionally substituted heterocyclyl, optionally substituted arylalkyl, or optionally substituted heteroarylalkyl;

R¹³ is independently for each occurrence halogen, CN, NH₂, or optionally substituted C₁-C₆ alkyl;

R¹⁴ is H, optionally substituted C₁-C₆ alkyl, optionally substituted C₂-C₆ alkenyl, optionally substituted C₂-C₆ alkynyl, optionally substituted aryl, optionally substituted heteroaryl, optionally substituted cyclyl, optionally substituted heterocyclyl, optionally substituted arylalkyl, optionally substituted heteroarylalkyl, or the two R¹⁴ taken together with the nitrogen atom to which they are attached form a 5-to-7 membered ring which optionally comprises 1-3-additional heteroatoms selected from N, O or S;

X₂ is N or CR¹⁵;

R¹⁵ is NHR¹⁶, optionally substituted C₁-C₆ alkyl, optionally substituted C₂-C₆ alkenyl, optionally substituted C₂-C₆ alkynyl, optionally substituted aryl, optionally substituted heteroaryl, optionally substituted cyclyl, optionally substituted heterocyclyl, optionally substituted arylalkyl, or optionally substituted heteroarylalkyl;

R¹⁶ is H, optionally substituted C₁-C₆ alkyl, optionally substituted C₂-C₆ alkenyl, optionally substituted C₂-C₆ alkynyl, optionally substituted aryl, optionally substituted heteroaryl, optionally substituted cyclyl, optionally substituted heterocyclyl, optionally substituted arylalkyl, or optionally substituted heteroarylalkyl;

Y₂ is N or CH;

q is 0, 1, 2, or 3; and

pharmaceutically acceptable salts and amides thereof.

In some embodiments, R⁹ is H or optionally substituted C₁-C₆ alkyl.

In preferred embodiments, R¹⁹ is H.

In some embodiments, R¹¹ is selected from the group consisting of H, optionally substituted aryl, optionally substituted heteroaryl, optionally substituted arylalkyl, and optionally substituted heteroarylalkyl. When R¹¹ is optionally substituted aryl, optionally substituted heteroaryl, optionally substituted arylalkyl or optionally substituted heteroarylalkyl. R¹⁴ can be selected from the group consisting of phenyl, thiophen-2-yl, 1-methyl-2-oxobenzoxazolin-5-yl, 2-(dimethylamino)-5-pyrimidinyl, 2-(N-formyl-N-methyl amino)-3-pyrimidinyl, 2-(N-(2-methoxyethyl)-N-methylamino)-5-pyrimidinyl, 5-dimethylamino-2-pyridinyl, 5-(N-(2-methoxyethyl)-N-methylamino)-2-pyridinyl, 2-(Nmethylamino)-5-pyrimidinyl, 2-(1-morpholinyl)-5pyrimidinyl, 2-(1-pyrrolidinyl)-5-pyrimidinyl, 2-dimethylamino-5-pyrimidinyl, 2-furanyl, 2-oxobenzoxazolin-5-yl, 2-pyridyl, 3-(dimethylamino)phenyl, 3-amino-4-methoxyphenyl, 3-bromo-4-(dimethylamino)phenyl, 3-methoxyphenyl, 3-methyl-4-(N-acetyl-N-methylamino)phenyl, 3-methyl-4-(N-formyl-Nmethylamino)phenyl, 3-methyl-4-(N-methyl-N-(trifluoroacetyl)amino)phenyl, 3-methyl-4-(N-methylamino)phenyl, 3-methyl-4-pyrrolidinylphenyl, 3-pyridyl, 3,4-dichlorophenyl, 3,4-methylenedioxyphenyl, 3,4,5-trimethoxyphenyl, 4-(acetylamino)phenyl, 4-(dimethylamino)-3-fluorophenyl, 4-(dimethylamino)phenyl, 4-(imidazol-1-yl)phenyl, 4-(methylthio)phenyl, 4-(morpholinyl)phenyl, 4-(N-(2-(dimethylamino)ethyl)amino)phenyl, 4-(N-(2-methoxyethyl)amino)phenyl, 4-(Nacetyl-N-methylamino)phenyl, 4-(N-ethyl-N-formylamino)phenyl, 4-(N-ethylamino)phenyl, 4-(N-formyl-N-(2-methoxyethyl)amino)phenyl, 4-(N-isopropylamino)phenyl, 4-(N-methyl-N-((2-dimethylamino)ethyl)amino)phenyl, 4-(N-methyl-N-(2-(N-phthalimidyl)acetyl)amino)phenyl, 4-(N-methyl-N-(2-cyano)ethylamino)phenyl, 4-(N-methylN-(2-methoxyethyl)amino)phenyl, 4-(N-methyl-N-(3-methoxy)propionylamino)phenyl, 4-(N-methyl-Nacetylamino)phenyl, 4(N-methyl-N-formylamino)phenyl, 4-(N-methyl-N-trifluoroacetylamino)phenyl, 4-(Nmorpholinyl)phenyl, 4-(thiophen-2-yl)phenyl, 4-(ureido)phenyl, 4-(2-(dimethylamino)acetylamino)phenyl, 4-(2-(2-methoxy)acetylamino)ethyl)amino)phenyl, 4-(2-methoxy)ethoxyphenyl, 4-(2-oxo-1-oxazolidinyl)phenyl, 4-(4-methoxy-2-butyl)phenyl, 4-(4-methylpiperidinyl)phenyl, 4-(5-pyrimidinyl)phenyl, 4-butoxyphenyl, 4-carboxamidophenyl, 4-chlorophenyl, 4-cyanophenyl, 4-diethylaminophenyl, 4-diethylmalonylallylphenyl), 4-dimethylaminophenyl, 4-ethoxyphenyl, 4-ethylphenyl, 4-hydroxyphenyl, 4-imidazolylphenyl, 4-iodophenyl, 4-isopropylphenyl, 4-methoxyphenyl, 4-methylaminophenyl, 4-methylsulfonylphenyl, 4-morpholinylphenyl, 4-N-(2-(dimethylamino)ethyl)-Nformylamino)phenyl, 4-N-(3-methoxypropionyl)-N-5 isopropyl-amino)phenyl, 4-N-ethyl-N-(2-methoxyethyl)amino)phenyl, 4-N-formylpiperidinylphenyl, 4-nitrophenyl, 4-piperidinylphenyl, 4-pyridylphenyl, 4-pyrrolidinylphenyl, 4-t-butylacrylphenyl, 5-(dimethylamino)thiophen-2-yl, 5-amino-2-pyridyl, 5-dimethylamino-2-pyrazinyl, 10 3-dimethylaminopyridazin-6-yl, 5-dimethylamino-2pyridyl, 5-pyrimidinylphenyl, 6-(N-methyl-Nformylamino)-3-pyridinyl, 6-(N-methyl-N-(2-methoxyethyl)amino)-3-pyridinyl, 6-(2-oxo-oxazolidinyl)-3-pyridinyl, 6-dimethylamino-3-pyridinyl, 6-imidazolyl-3-pyridinyl, 6-morpholinyl-3-pyridinyl, 6-pyrrolidinyl-3pyridinyl, (2-propyl)-3-pyridinyl, and (4-formylamino)phenyl, (thiophen-2-yl)methyl, (thiophen-3-yl)methyl, butyl, cycloheptyl, pentyl, thiophen-2-yl, 1-(3-bromophenyl)ethyl, 2-(N-phenylmethoxycarbonyl)aminophenyl, 2-(3-bromophenyl)ethyl, 2-(3-cyanophenyl)methyl, 2-(4-bromophenyl)ethyl, 2-(5-chloro-2-(thiophen3-yl)phenyl, 2-bromophenyl, 2-furanyl, 2-methylpropyl, 2-phenylethyl, phenylmethyl, 2,3-dimethoxyphenyl, 2,3-methylenedioxyphenyl, 3-(furan-2-yl)phenyl, 3-(thiophen-2-yl)phenyl, 3-(2-pyridyl)phenyl, 3-(3-methoxybenzyl)phenyl, 3-(amino)propynyl, 3-benzyloxyphenyl, 3-bromo-4-fluorophenyl, 3-bromo-5-iodophenyl, 3-bromo-5-methoxyphenyl, 3-bromophenyl, 3-bromophenylmethyl, 3-carboxamidophenyl, 3-chlorophenyl, 3-cyanophenyl, 3-diethylmalonylallylphenyl, 3-dimethylaminophenyl, 3-ethoxyphenyl, 3-fluoro-5-trifluoromethylphenyl, 3-fluorophenyl, 3-hydroxyphenyl, 3-iodophenyl, 3-methoxyethyoxyphenyl, 3-methoxyphenyl, 3-methylphenyl, 3-methylsulfonylphenyl, 3-methylthiophenyl, 3-t-butylacrylphenyl, 3-trifloromethyoxyphenyl, 3-trifluoromethylphenyl, 3-vinylpyridinylphenyl, 3,4-dichlorophenyl, 3,4-dimethoxyphenyl, 3,4-methylenedioxyphenyl, 3,4,5trimethoxyphenyl, 3,5-di(trifluoromethyl)phenyl, 3,5-dibromophenyl, 3,5-dichlorophenyl, 3,5-dimethoxyphenyl, 3,5-dimethylphenyl, 4(2-propyl)phenyl, 4-(2-propyl)oxyphenyl, 4-benzyloxyphenyl, 4-bromophenyl, 4-bromothiophen-2-yl, 4-butoxyphenyl, 4-dimethylaminophenyl, 4-fluoro-3-trifluoromethylphenyl, 4-methoxyphenyl, 4-neopentylphenyl, 4-phenoxyphenyl, 5-bromothiophen-2-yl, cyclohexyl, cyclopropyl, hexyl, methyl, phenyl, (2-bromo-5-chlorophenyl)methyl, (2-bromophenyl)methyl, 6-cyclopropylmethylamino-3-pyridinyl, and (5-chloro-2-(3-methoxyphenyl)phenyl)methyl. In some embodiments, R¹¹ is H.

In some embodiments, R¹² is selected from the group consisting of N(R¹⁴)₂, OR¹⁴, optionally substituted aryl, optionally substituted heteroaryl, optionally substituted arylalkyl, and optionally substituted heteroarylalkyl. When R¹² is N(R¹⁴)₂, one or both R¹⁴ can be H. Preferably at least one R¹⁴ is not H, e.g., one R¹⁴ is H and the other R¹⁴ is optionally substituted C₁-C₆ alkyl, optionally substituted aryl, optionally substituted heteroaryl, optionally substituted arylalkyl or optionally substituted heteroarylalkyl, most preferably both R¹⁴ are independently C₁-C₆ alkyl. In some embodiments, R¹² is selected from the group consisting of dialkylamino (e.g. dimethylamino) and optionally substituted 5-8 membered heterocyclyl, wherein the heterocyclyl comprises at least one nitrogen atom (e.g. morpholine, pyridine).

In some preferred embodiments, q is 0.

In some embodiments, R¹⁴ is an optionally substituted C₁-C₆ alkyl, e.g., optionally substituted methyl or optionally substituted ethyl. The C₁-C₆ alkyl can be substituted with C₁-C₆ alkyl, C₂-C₆ alkenyl, aryl, or cyclyl, each which can also be optionally substituted. In some embodiments, R¹⁴ is a substituted methyl or substituted ethyl, wherein the methyl and or ethyl is substituted with an optionally substituted aryl. In some embodiments, R¹⁴ is cyclyl, e.g. cyclopentane, cyclohexane, or cycloheptane. Preferably cyclyl is cyclohexane. In some embodiments, R¹⁴ is heterocyclyl, preferably heterocyclyl comprises at least one O or N atom. In some other embodiments, R¹⁴ is optionally substituted aryl. For example, optionally substituted phenyl, e.g., an aryl substituted with at least one halogen.

In some embodiments, X₂ is CR¹⁵ and R¹⁵ is selected from the group consisting of NHR¹⁶, optionally substituted C₁-C₆ alkyl, optionally substituted C₂-C₆ alkenyl, optionally substituted C₂-C₆ alkynyl, optionally substituted aryl, optionally substituted heteroaryl, optionally substituted cyclyl, optionally substituted heterocyclyl, optionally substituted arylalkyl, and optionally substituted heteroarylalkyl. Preferably R¹⁶ is an optionally substituted alkyl, e.g., optionally substituted methyl, optionally substituted ethyl, for example methyl substituted with aryl or heteroaryl, e.g., indolyl.

In some embodiments, R¹⁵ is selected from the group consisting of phenyl, thiophen-2-yl, 1-methyl-2-oxobenzoxazolin-5-yl, 2-(dimethylamino)-5-pyrimidinyl, 2-(N-formyl-N-methyl amino)-3-pyrimidinyl, 2-(N-(2-methoxyethyl)-N-methylamino)-5-pyrimidinyl, 5-dimethylamino-2-pyridinyl, 5-(N-(2-methoxyethyl)-N-methylamino)-2-pyridinyl, 2-(Nmethylamino)-5-pyrimidinyl, 2-(1-morpholinyl)-5pyrimidinyl, 2-(1-pyrrolidinyl)-5-pyrimidinyl, 2-dimethylamino-5-pyrimidinyl, 2-furanyl, 2-oxobenzoxazolin-5-yl, 2-pyridyl, 3-(dimethylamino)phenyl, 3-amino-4-methoxyphenyl, 3-bromo-4-(dimethylamino)phenyl, 3-methoxyphenyl, 3-methyl-4-(N-acetyl-N-methylamino)phenyl, 3-methyl-4-(N-formyl-Nmethylamino)phenyl, 3-methyl-4-(N-methyl-N-(trifluoroacetyl)amino)phenyl, 3-methyl-4-(N-methylamino)phenyl, 3-methyl-4-pyrrolidinylphenyl, 3-pyridyl, 3,4-dichlorophenyl, 3,4-methylenedioxyphenyl, 3,4,5-trimethoxyphenyl, 4-(acetylamino)phenyl, 4-(dimethylamino)-3-fluorophenyl, 4-(dimethylamino)phenyl, 4-(imidazol-1-yl)phenyl, 4-(methylthio)phenyl, 4-(morpholinyl)phenyl, 4-(N-(2-(dimethylamino)ethyl)amino)phenyl, 4-(N-(2-methoxyethyl)amino)phenyl, 4-(Nacetyl-N-methylamino)phenyl, 4-(N-ethyl-N-formylamino)phenyl, 4-(N-ethylamino)phenyl, 4-(N-formyl-N-(2-methoxyethyl)amino)phenyl, 4-(N-isopropylamino)phenyl, 4-(N-methyl-N-((2-dimethylamino)ethyl)amino)phenyl, 4-(N-methyl-N-(2-(N-phthalimidyl)acetyl)amino)phenyl, 4-(N-methyl-N-(2-cyano)ethylamino)phenyl, 4-(N-methylN-(2-methoxyethyl)amino)phenyl, 4-(N-methyl-N-(3-methoxy)propionylamino)phenyl, 4-(N-methyl-Nacetylamino)phenyl, 4(N-methyl-N-formylamino)phenyl, 4-(N-methyl-N-trifluoroacetylamino)phenyl, 4-(N-morpholinyl)phenyl, 4-(thiophen-2-yl)phenyl, 4-(ureido)phenyl, 4-(2-(dimethylamino)acetylamino)phenyl, 4-(2-(2-methoxy)acetylamino)ethyl)amino)phenyl, 4-(2-methoxy)ethoxyphenyl, 4-(2-oxo-1-oxazolidinyl)phenyl, 4-(4-methoxy-2-butyl)phenyl, 4-(4-methylpiperidinyl)phenyl, 4-(5-pyrimidinyl)phenyl, 4-butoxyphenyl, 4-carboxamidophenyl, 4-chlorophenyl, 4-cyanophenyl, 4-diethylaminophenyl, 4-diethylmalonylallylphenyl), 4-dimethylaminophenyl, 4-ethoxyphenyl, 4-ethylphenyl, 4-hydroxyphenyl, 4-imidazolylphenyl, 4-iodophenyl, 4-isopropylphenyl, 4-methoxyphenyl, 4-methylaminophenyl, 4-methylsulfonylphenyl, 4-morpholinylphenyl, 4-N-(2-(dimethylamino)ethyl)-Nformylamino)phenyl, 4-N-(3-methoxypropionyl)-N-5 isopropyl-amino)phenyl, 4-N-ethyl-N-(2-methoxyethyl)amino)phenyl, 4-N-formylpiperidinylphenyl, 4-nitrophenyl, 4-piperidinylphenyl, 4-pyridylphenyl, 4-pyrrolidinylphenyl, 4-t-butylacrylphenyl, 5-(dimethylamino)thiophen-2-yl, 5-amino-2-pyridyl, 5-dimethylamino-2-pyrazinyl, 10 3-dimethylaminopyridazin-6-yl, 5-dimethylamino-2-pyridyl, 5-pyrimidinylphenyl, 6-(N-methyl-Nformylamino)-3-pyridinyl, 6-(N-methyl-N-(2-methoxyethyl)amino)-3-pyridinyl, 6-(2-oxo-oxazolidinyl)-3-pyridinyl, 6-dimethylamino-3-pyridinyl, 6-imidazolyl-3-pyridinyl, 6-morpholinyl-3-pyridinyl, 6-pyrrolidinyl-3pyridinyl, (2-propyl)-3-pyridinyl, and (4-formylamino)phenyl, (thiophen-2-yl)methyl, (thiophen-3-yl)methyl, butyl, cycloheptyl, pentyl, thiophen-2-yl, 1-(3-bromophenyl)ethyl, 2-(N-phenylmethoxycarbonyl)aminophenyl, 2-(3-bromophenyl)ethyl, 2-(3-cyanophenyl)methyl, 2-(4-bromophenyl)ethyl, 2-(5-chloro-2-(thiophen3-yl)phenyl, 2-bromophenyl, 2-furanyl, 2-methylpropyl, 2-phenylethyl, phenylmethyl, 2,3-dimethoxyphenyl, 2,3-methylenedioxyphenyl, 3-(furan-2-yl)phenyl, 3-(thiophen-2-yl)phenyl, 3-(2-pyridyl)phenyl, 3-(3-methoxybenzyl)phenyl, 3-(amino)propynyl, 3-benzyloxyphenyl, 3-bromo-4-fluorophenyl, 3-bromo-5-iodophenyl, 3-bromo-5-methoxyphenyl, 3-bromophenyl, 3-bromophenylmethyl, 3-carboxamidophenyl, 3-chlorophenyl, 3-cyanophenyl, 3-diethylmalonylallylphenyl, 3-dimethylaminophenyl, 3-ethoxyphenyl, 3-fluoro-5-trifluoromethylphenyl, 3-fluorophenyl, 3-hydroxyphenyl, 3-iodophenyl, 3-methoxyethyoxyphenyl, 3-methoxyphenyl, 3-methylphenyl, 3-methylsulfonylphenyl, 3-methylthiophenyl, 3-t-butylacrylphenyl, 3-trifloromethyoxyphenyl, 3-trifluoromethylphenyl, 3-vinylpyridinylphenyl, 3,4-dichlorophenyl, 3,4-dimethoxyphenyl, 3,4-methylenedioxyphenyl, 3,4,5-trimethoxyphenyl, 3,5-di(trifluoromethyl)phenyl, 3,5-dibromophenyl, 3,5-dichlorophenyl, 3,5-dimethoxyphenyl, 3,5-dimethylphenyl, 4(2-propyl)phenyl, 4-(2-propyl)oxyphenyl, 4-benzyloxyphenyl, 4-bromophenyl, 4-bromothiophen-2-yl, 4-butoxyphenyl, 4-dimethylaminophenyl, 4-fluoro-3-trifluoromethylphenyl, 4-methoxyphenyl, 4-neopentylphenyl, 4-phenoxyphenyl, 5-bromothiophen-2-yl, cyclohexyl, cyclopropyl, hexyl, methyl, phenyl, (2-bromo-5-chlorophenyl)methyl, (2-bromophenyl)methyl, 6-cyclopropylmethylamino-3-pyridinyl, (5-chloro-2-(3-methoxyphenyl)phenyl)methyl, 2-bromophenyl, 3-bromophenyl, 4-bromophenyl, (2-bromophenyl)methyl, 2-chlorophenyl, 3-chlorophenyl, 4-chlorophenyl, 2-fluorophenyl, 3-fluorophenyl, 4-fluorophenyl, 2-nitrophenyl, 3-nitrophenyl, 4-nitrophenyl, 2-methoxyphenyl, 3-methoxyphenyl, 4-methoxyphenyl, 3-aminophenyl, 3-aminophenyl, 4-aminophenyl, cyclohexane, tetrahydropyran (e.g., tetrahydropyran-4-yl), 1-(2-bromophenyl)ethyl, 4-methylpent-1-en-4-yl, and (indol-2-yl-methyl)amino.

In some embodiments, Y² is N.

In some embodiments, the compound of formula (II) is ABT-702 (5-(3-bromophenyl)-7-(6-morpholinopyridin-3-yl)pyrido[2,3-d]pyrimidin-4-amine, also referred to as B8 herein), compound 6 (7-(4-(dimethylamino)phenyl)pteridin-4-amine), compound 7 (5-(3-bromophenyl)-7-(4-(dimethylamino)phenyl)pyrido[2,3-d]pyrimidin-4-amine), compound 8 (5-(2-bromobenzyl)-7-(6-morpholinopyridin-3-yl)pyrido[2,3-d]pyrimidin-4-amine), compound 9 (5-cyclohexyl-7-(6-morpholinopyridin-3-yl)pyrido[2,3-d]pyrimidin-4-amine), compound 10 (5-(tetrahydro-2H-pyran-4-yl)-7-(6-morpholinopyridin-3-yl)pyrido[2,3-d]pyrimidin-4-amine), compound 11 (5-(1-(2-bromophenyl)ethyl)-7-(6-morpholinopyridin-3-yl)pyrido[2,3-d]pyrimidin-4-amine), compound 12 (5-(2-methylpent-4-en-2-yl)-7-(6-morpholinopyridin-3-yl)pyrido[2,3-d]pyrimidin-4-amine), or compound 13 (N-5-((1H-indol-3-yl)methyl)-7-(6-morpholinopyridin-3-yl)pyrido[2,3-d]pyrimidine-4,5-diamine).

In some embodiments, the compound of formula (II) is selected from the group consisting of 4-amino-5-(4-chlorophenyl)-7-(4-nitrophenyl)pyrido[2,3-d]pyrimidine; 4-amino-5-(4-methoxyphenyl)-7-(4-nitrophenyl)pyrido[2,3-d]pyrimidine; 4-amino-5-(4-fluorophenyl)-7-(4-fluorophenyl)pyrido[2,3-d]pyrimidine; 4-amino-5-(4-chlorphenyl)-7-(4-fluorphenyl)pyrido[2,3-d]pyrimidine; 4-amino-5-phenyl-7-(4-aminophenyl)pyrido[2,3-d]pyrimidine; 4-amino-5-phenyl-7-(4-bromphenyl)pyrido[2,3-d]pyrimidine; 4-amino-5-(4-methoxyphenyl)-7-(4-aminophenyl)pyrido[2,3-d]pyrimidine; 4-amino-5,7-diphenylpyrido[2,3-d]pyrimidine; 4-amino-5-(4-dimethylaminophenyl)-7-(4-bromophenyl)pyrido[2,3-d]pyrimidine; 4-amino-5-(4-dimethylaminophenyl)-7-(4-dimethylaminophenyl)pyrido[2,3-d]pyrimidine; 4-amino-S-(4-ethoxyphenyl)-7-(4-dimethylaminophenyl)pyrido[2,3-d]pyrimidine; 4-amino-5-(4-dimethylaminophenyl)-7-(4-methoxyphenyl)pyrido[2,3-d]pyrimidine; 4-amino-5-(4-(2-propyl)phenyl)-7-(4-methoxyphenyl)pyrido[2,3-d]pyrimidine; 4-amino-5-(4-neopentylphenyl)-7-(4-methoxyphenyl)pyrido[2,3-d]pyrimidine; 4-amino-5-(4-butyloxyphenyl)-7-(4-methoxyphenyl)pyrido[2,3-d]pyrimidine; 4-amino-5-(4-methoxyphenyl)-7-(4-bromophenyl)pyrido[2,3-d]pyrimidine; 4-amino-5-(4-(2-propyl)oxyphenyl)-7-(4-methoxyphenyl)pyrido[2,3-d]pyrimidine; 4-amino-5-(4-butoxyphenyl)-7-(4-N-formylpiperazinylphenyl)pyrido[2,3-d]pyrimidine; 4-amino-5-(4-benzylOxyphenyl)-7-(4-methoxyphenyl)pyrido[2,3-d]pyrimidine; 4-amino-5-(4-phenoxyphenyl)-7-(4-methoxyphenyl)pyrido[2,3-d]pyrimidine; 4-amino-5-(4-(2-propyl)phenyl)-7-(4-diethylmalonylallylphenyl)pyrido[2,3-d]pyrimidine; 4-amino-5-(4-(2-propyl)phenyl)-7-(4-t-butylacrylphenyl)pyrido[2,3-d]pyrimidine; 4-amino-5-(3-bromophenyl)-7-(4-dimethylaminophenyl)pyrido[2,3-d]pyrimidine; 4-amino-5-(3,4-dimethoxyphenyl)-7-(4-dimethylaminophenyl)pyrido[2,3-d]pyrimidine; 4-amino-5-(3-t-butylacrylphenyl)-7-(4-dimethylaminophenyl)pyrido[2,3-d]pyrimidine; 4-amino-5-(3-methoxyphenyl-7-(4-dimethylaminophenyl)pyrido[2,3-d]pyrimidine; 4-amino-5-(3,5-dimethoxyphenyl-7-(4-dimethylaminophenyl)pyrido[2,3-d]pyrimidine; 4-amino-5-(3-diethylmalonylallylphenyl)-7-(4-dimethylaminophenyl)pyrido[2,3-d]pyrimidine; 4-amino-5-(3-vinylpyridinylphenyl)-7-(4-dimethylaminophenyl)pyrido[2,3-d]pyrimidine; 4-amino-5-(3-trifluoromethylphenyl)-7-(4-dimethylaminophenyl)pyrido[2,3-d]pyrimidine; 4-amino-5-(3-carboxamidophenyl)-7-(4-dimethylaminophenyl)pyrido[2,3-d]pyrimidine; 4-amino-5-(3-cyanophenyl)-7-(4-dimethylaminophenyl)pyrido[2,3-d]pyrimidine; 4-amino-5-(3-benzyloxyphenyl)-7-(4-dimethylaminophenyl)pyrido[2,3-d]pyrimidine; 4-amino-5-(3-methoxyphenyl)-7-(4-methoxyphenyl)pyrido[2,3-d]pyrimidine; 4-amino-5-(3-bromophenyl)-7-(4-butoxyphenyl)pyrido[2,3-d]pyrimidine; 4-amino-5-(3-(2-pyridyl)phenyl)-7-(4-dimethylaminophenyl)pyrido[2,3-d]pyrimidine; 4-amino-5-(3-methylphenyl)-7-(4-dimethylaminophenyl)pyrido[2,3-d]pyrimidine; 4-amino-5-(3-chlorophenyl)-7-(4-dimethylaminophenyl)pyrido[2,3-d]pyrimidine; 4-amino-5-(3-fluorophenyl)-7-(4-dimethylaminophenyl)pyrido[2,3-d]pyrimidine; 4-amino-5-(3-bromophenyl)-7-(4-methoxyphenyl)pyrido[2,3-d]pyrimidine; 4-amino-5-(3-methoxyphenyl)-7-(4-bromophenyl)pyrido[2,3-d]pyrimidine; 4-amino-5-(3-bromophenyl)-7-phenylpyrido[2,3-d]pyrimidine; 4-amino-5-(3-bromophenyl)-7-(4-ethylphenyl)pyrido[2,3-d]pyrimidine; 4-amino-5-(3-bromophenyl)-7-(4-bromophenyl)pyrido[2,3-d]pyrimidine; 4-amino-5-(3-bromophenyl)-7-(4-cyanophenyl)pyrido[2,3-d]pyrimidine; 4-amino-5-(3-bromophenyl)-7-(4-hydroxyphenyl)pyrido[2,3-d]pyrimidine; 4-amino-5-(3-iodophenyl)-7-(4-dimethylaminophenyl)pyrido[2,3-d]pyrimidine; 4-amino-5-(3-ethoxyphenyl)-7-(4-dimethylaminophenyl)pyrido[2,3-d]pyrimidine; 4-amino-5-(3-trifluoromethyloxyphenyl)-7-(4-dimethylaminophenyl)pyrido[2,3-d]pyrimidine; 4-amino-5-(3,5-dichlorophenyl)-7-(4-dimethylaminophenyl)pyrido[2,3-d]pyrimidine; 4-amino-5-(3-bromo-4-fluorophenyl)-7-(4-dimethylaminophenyl)pyrido[2,3-d]pyrimidine; 4-amino-5-(3-hydroxyphenyl)-7-(4-dimethylaminophenyl)pyrido[2,3-d]pyrimidine; 4-amino-5-(3-bromophenyl)-7-(4-morpholinylphenyl)pyrido[2,3-d]pyrimidine; 4-amino-5-(3-bromophenyl)-7-(4-piperidinylphenyl)pyrido[2,3-d]pyrimidine; 4-amino-5-(3-bromophenyl)-7-(4-(imidazol 1-yl)phenyl)pyrido[2,3-d]pyrimidine; 4-amino-5-(3-bromophenyl)-7-(4-chlorophenyl)pyrido[2,3-d]pyrimidine; 4-amino-5-(3-bromophenyl)-7-(4-isopropylphenyl)pyrido[2,3-d]pyrimidine; 4-amino-S-(3-bromophenyl)-7-(4-trifluorophenyl)pyrido[2,3-d]pyrimidine; 4-amino-5-(3-bromophenyl)-7-(4-diethylaminophenyl)pyrido[2,3-d]pyrimidine; 4-amino-5-(3-bromophenyl)-7-(3,4,5-trimethoxyphenyl)pyrido[2,3-d]pyrimidine; 4-amino-5-(3-(3-methoxybenzyl)phenyl)-7-(4-dimethylaminophenyl)pyrido[2,3-d]pyrimidine; 4-amino-5-(3-methoxyethyoxyphenyl)-7-(4-dimethylaminophenyl)pyrido[2,3-d]pyrimidine; 4-amino-5-(3,4-methylenedioxyphenyl)-7-(4-dimethylaminophenyl)pyrido[2,3-d]pyrimidine; 4-amino-5-(3-bromophenyl)-7-(4-ethoxyphenyl)pyrido[2,3-d]pyrimidine; 4-amino-5-(3-bromophenyl)-7-(thiophen-2-yl)pyrido[2,3-d]pyrimidine; 4-amino-5-(3-bromophenyl)-7-(4-fluorophenyl)pyrido[2,3-d]pyrimidine; 4-amino-5-(3-dimethylaminophenyl)-7-(4-dimethylaminophenyl)pyrido[2,3-d]pyrimidine; 4-amino-5-phenyl-7-(4-dimethylaminophenyl)pyrido[2,3-d]pyrimidine; 4-amino-5-(3,4,5-trimethoxyphenyl)-7-(4-dimethylaminophenyl)pyrido[2,3-d]pyrimidine; 4-amino-5-(3-bromophenyl)-7-(4-nitrophenyl)pyrido[2,3-d]pyrimidine; 4-amino-5-(3-bromophenyl)-7-(4-iodophenyl)pyrido[2,3-d]pyrimidine; 4-amino-5-(3-bromophenyl)-7-(3,4-methylenedioxophenyl)pyrido[2,3-d]pyrimidine; 4-amino-5-(thiophen-2-yl)-7-(4-morpholinylphenyl)pyrido[2,3-d]pyrimidine; 4-amino-5-(3,5-dimethoxyphenyl)-7-(thiophen-2-yl)pyrido[2,3-d]pyrimidine; 4-amino-5-(3-bromophenyl)-7-(4-carboxamidophenyl)pyrido[2,3-d]pyrimidine; 4-amino-5-(3-bromophenyl)-7-(4-(2-methoxy)ethoxyphenyl)pyrido[2,3-d]pyrimidine; 4-amino-5-(3,5-dimethoxyphenyl)-7-(4-morpholinylphenyl)pyrido[2,3-d]pyrimidine; 4-amino-5-(3-trifluoromethylphenyl)-7-(thiophene-2-yl)pyrido[2,3-d]pyrimidine; 4-amino-5-(3-bromophenyl)-7-(4-aminophenyl)pyrido[2,3-d]pyrimidine; 4-amino-5-(3-bromo-4-fluorophenyl)-7-(thiophene-2-yl)pyrido[2,3-d]pyrimidine; 4-amino-5-(3-bromo-4-fluorophenyl)-7-(2-furanyl)pyrido[2,3-d]pyrimidine; 4-amino-5-(3,5-dimethoxyphenyl)-7-(4-iodophenyl)pyrido[2,3-d]pyrimidine; 4-amino-5-(3,5-dimethoxyphenyl)-7-(4-imidazolylphenyl)pyrido[2,3-d]pyrimidine; 4-amino-5-(3,5-dimethoxyphenyl)-7-(4-(thiophene-2-yl)phenyl)pyrido[2,3-d]pyrimidine; 4-amino-5-(3,5-dimethoxyphenyl)-7-(4-(3-pyridyl)phenyl)pyrido[2,3-d]pyrimidine; 4-amino-5-(3-bromophenyl)-7-(4-(4-methylpiperidinyl)phenyl)pyrido[2,3-d]pyrimidine; 4-amino-5-(3-bromophenyl)-7-(4-pyrrolidinylphenyl)pyrido[2,3-d]pyrimidine; 4-amino-5-(4-bromothiophene-)-7-(4-dimethylaminophenyl)pyrido[2,3-d]pyrimidine; 4-amino-5-(4-bromothiophene-2-yl)-7-(4-morpholinylphenyl)pyrido[2,3-d]pyrimidine; 4-morpholinyl-5-(3-bromophenyl)-7-(4-dimethylaminophenyl)pyrido[2,3-d]pyrimidine; 4-amino-5 (5-bromothiophene-2-yl)-7-(4-morpholinylphenyl)pyrido[2,3-d]pyrimidine; 4-amino-5-(4-bromophenyl)-7-(4-dimethylaminophenyl)pyrido[2,3-d]pyrimidine; 4-amino-5-(3-bromophenyl)-7-(4-(acetylamino)phenyl)pyrido[2,3-d]pyrimidine; 4-amino-5-(3-bromophenyl)-7-(4-dimethylaminophenyl)pyrido[2,3-d]pyrimidine; 4-amino-5-(3,5-dimethoxyphenyl)-7-(5-pyrimidinylphenyl)pyrido[2,3-d]pyrimidine; 4-(4-fluorophenyl)amino)-5-(3-bromophenyl)-7-(4-dimethylaminophenyl)pyrido[2,3-d]pyrimidine; 4-amino-5-(4-bromothiophene-2-yl)-7-(4-pyrrolidinylphenyl)pyrido[2,3-d]pyrimidine; 4-amino-5-(4-bromothiophene-2-yl)-7-(thiophene-2-yl)pyrido[2,3-d]pyrimidine; 4-amino-5-(3-bromophenyl)-7-(5-(dimethylamino)thiophene-2-yl)pyrido[2,3-d]pyrimidine; 4-amino-5-(3-bromo-5-iodophenyl)-7-(4-(dimethylamino)phenyl)pyrido[2,3-d]pyrimidine; 4-amino-5-(3,5-di(trifluoromethyl)phenyl)-7-(4-(dimethylamino)phenyl)pyrido[2,3-d]pyrimidine; 4-amino-5-(3,5-di(trifluoromethyl)phenyl)-7-(4-morpholinylphenyl)pyrido[2,3-d]pyrimidine; 4-amino-5-(3,5-dibromophenyl)-7-(4-(dimethylamino)phenyl)pyrido[2,3-d]pyrimidine; 4-amino-5-(3,S-dibromophenyl)-7-(4-morpholinylphenyl)pyrido[2,3-d]pyrimidine; 4-amino-5-(4-bromothiophene-2-yl)-7-(4-(4-methylpiperidinyl)phenyl)pyrido[2,3-d]pyrimidine; 4-amino-5-(3,5-dibromophenyl)-7-(4(dimethylamino)phen)pyrido[2,3d]pyrimidine; 4-amino-5-(3-bromophenyl)-7-(3-(dimethylamino)phenyl)pyrido[2,3-d]pyrimidine; 4-amino-5-(3-bromophenyl)-7-(4-methylsulfonylphenyl)pyrido[2,3-d]pyrimidine; 4-amino-5-(3-bromophenyl)-7-(3-methoxyphenyl)pyrido[2,3-d]pyrimidine; 4-amino-5-(3-bromophenyl)-7-(4-(methylthio)phenyl)pyrido[2,3-d]pyrimidine; 4-amino-5-(3-bromophenyl)-7-(3,4-dichlorophenyl)pyrido[2,3-d]pyrimidine; 4-amino-5-(3-bromophenyl)-7-(4-(N-methyl-N-formylamino)phenyl)pyrido[2,3-d]pyrimidine; 4-amino-5-(3-bromophenyl)-7-(4-methylaminophenyl)pyrido[2,3-d]pyrimidine; 4-amino-5-(3-bromo-4-fluorophenyl)-7-(4-methylSulfonylphenyl)pyrido[2,3-d]pyrimidine; 4-amino-5-(3-bromophenyl)-7-(3-amino-4-methoxyphenyl)pyrido[2,3-d]pyrimidine; 4-amino-5-(3-bromophenyl)-7-(3-bromo-4-(dimethylamino)phenyl)pyrido[2,3-d]pyrimidine; 4-amino-5-(3-bromophenyl)-7-(3-methyl-4-(dimethylamino)phenyl)pyrido[2,3-d]pyrimidine; 4-amino-5-(3-bromophenyl)-7-(4-(N-methyl-N-trifluoroacetylamino)phenyl)pyrido[2,3-d]pyrimidine; 4-amino-5-(3-bromophenyl)-7-(4-(dimethylamino)-3-fluorophenyl)pyrido[2,3-d]pyrimidine; 4-amino-5-(3-bromophenyl)-7-(4-(N-ethyl-N-formylamino)phenyl)pyrido[2,3d]pyrimidine; 4,4-bis(acetylamino)-5-(3-bromophenyl)-7-(4-(N-methyl-N-acetylamino)phenyl)pyrido[2,3-d]pyrimidine; 4-amino-5-(3-bromophenyl)-7-(4-(N-acetyl-N-methylamino)phenyl)pyrido[2,3-d]pyrimidine; 4-amino-5-(3-bromophenyl)-7-(4-(N-ethylamino)phenyl)pyrido[2,3-d]pyrimidine; 4-amino-5-(3-bromophenyl)-7-(4-(N-methyl-N-(2-methoxyethyl)amino)phenyl)pyrido[2,3-d]pyrimidine; 4-amino-5-(3-bromophenyl)-7-(4-(N-isopropylamino)phenyl)pyrido[2,3-d]pyrimidine; 4-amino-5-(3-bromophenyl)-7-(4-N-ethyl-N-(2-methoxyethyl)amino)phenyl)pyrido[2,3-d]pyrimidine; 4-amino-5-(3-bromophenyl)-7-(4-N-(3-methoxypropionyl)-N-isopropyl-amino)phenyl)pyrido[2,3-d]pyrimidine; 4-amino-5-(3-bromophenyl)-7-(4-N-(2-(dimethylamino)ethyl)-N-formylamino)phenyl)pyrido[2,3-d]pyrimidine; 4-amino-5-(3-bromophenyl)-7-(4-(N-(2-(dimethylamino)ethyl)amino)phenyl)pyrido[2,3-d]pyrimidine; 4-amino-5-(3-bromophenyl)-7-(4-(N-methyl-N-(2-cyano)ethylamino)phenyl)pyrido[2,3-d]pyrimidine; 4-amino-5-(3-bromophenyl)-7-(4-(N-methyl-N-(3-methoxy)propionylamino)phenyl)pyrido[2,3-d]pyrimidine; 4-amino-5-(3-bromophenyl)-7-(3-methyl-4-(N-formyl-N-methylamino)phenyl)pyrido[2,3-d]pyrimidine; 4-amino-5-(3-bromophenyl)-7-(3-methyl-4-(N-methylamino)phenyl)pyrido[2,3-d]pyrimidine; 4-amino-5-(3-bromophenyl)-7-(4-(4-methoxy-2-butyl)phenyl)pyrido[2,3-d]pyrimidine; 4-amino-5-(3-bromophenyl)-7-(4-(N-methyl-N-(2-(N-phthalimidyl)acetyl)amino)phenyl)pyrido[2,3-d]pyrimidine; 4-amino-5-(3-bromophenyl)-7-(3-methyl-4-(N-methyl-N-(trifluoroacetyl)amino)phenyl)pyrido[2,3-d]pyrimidine; 4-amino-5-(3-bromophenyl)-7-(3-methyl-4-(N-acetyl-N-methylamino)phenyl)pyrido[2,3-d]pyrimidine; 4-amino-5-(3-bromophenyl)-7-(6-dimethylamino-3-pyridinyl)pyrido[2,3-d]pyrimidine; 4-amino-5-(3-cyanophenyl)-7-(4-methylsulfonylphenyl)pyrido[2,3-d]pyrimidine; 4-amino-5-(3-cyanophenyl)-7-(4-(N-methyl-N-formylamino)-phenyl)pyrido[2,3-d]pyrimidine; 4-amino-5-(3-bromophenyl)-7-(6-(N-methyl-N-formylamino)-3-pyridinyl)pyrido[2,3-d]pyrimidine; 4-amino-5-(3-bromophenyl)-7-(6-morpholinyl-3-pyridinyl)pyrido[2,3-d]pyrimidine; 4-amino-5-(3-bromophenyl)-7-(6-(N-methyl-N-methoxyethylamino)-3-pyridinyl)pyrido[2,3-d]pyrimidine; 4-amino-5-(3-bromophenyl)-7-(6-pyrrolidinyl-3-pyridinyl)pyrido[2,3-d]pyrimidine; 4-amino-5-(3-bromophenyl)-7-(2-(dimethylamino)-5-pyrimidinyl)pyrido[2,3-d]pyrimidine; 4-amino-5-(3-bromophenyl)-7-(2-(N-methoxyethyl-N-methylamino)-5-pyrimidinyl)pyrido[2,3-d]pyrimidine; 4-amino-5-(3-bromophenyl)-7-(2-(N-formyl-N-methylamino)-5-pyrimidinyl)pyrido[2,3-d]pyrimidine; 4-amino-S-(3-bromophenyl)-7-(2-(N-methylamino)5-pyrimidinyl)pyrido[2,3-d]pyrimidine; 4-amino-5-(3-bromophenyl)-7-(2-(1-pyrrolidinyl)-5-pyrimidinyl)pyrido[2,3d]pyrimidine; 4-amino-5-(3-bromophenyl)-7-(2-(1-morpholinyl)-5-pyrimidinyl)pyrido[2,3-d]pyrimidine; 4-amino-5-(3-bromophenyl)-7-(6-(2-oxo-3-oxazolidinyl)-3-pyridinyl)pyrido[2,3-d]pyrimidine; 4-amino-5-(3-bromophenyl)-7-(2-pyridyl)pyrido[2,3-d]pyrimidine; 4-amino-5-(3-bromophenyl)-7-(3-pyridyl)pyrido[2,3-d]pyrimidine; 4-amino-5-(3-(thiophen-2-yl)phenyl)-7-(4-dimethylaminophenyl)pyrido[2,3-d]pyrimidine; 4-amino-5-(3-(furan-2-yl)phenyl)-7-(4-dimethylaminophenyl)pyrido[2,3-d]pyrimidine; 4-amino-5-(3-(3-methoxyphenyl)phenyl)-7-(4-dimethylaminophenyl)pyrido[2,3-d]pyrimidine; 4-amino-5-phenyl-7-(4-dimethylaminophenyl)pyrido[2,3-d]pyrimidine; 4-amino-5-(3-chlorophenyl)-7-(4-(morpholinyl)phenyl)pyrido[2,3-d]pyrimidine; 4-amino-5-(3-bromo-4-fluorophenyl)-7-(4-(morpholinyl)phenyl)pyrido[2,3-d]pyrimidine; 4-amino-5-(3-chlorophenyl)-7-(4-iodophenyl)pyrido[2,3-d]pyrimidine; 4-amino-5-(3-chlorophenyl)-7-(4-(thiophen-2-yl)phenyl)pyrido[2,3-d]pyrimidine; 4-amino-5-(3-chlorophenyl)-7-(4-(5-pyrimidinyl)phenyl)pyrido[2,3-d]pyrimidine; 4-amino-5-(3-bromo-4-fluorophenyl)-7-(4-iodophenyl)pyrido[2,3-d]pyrimidine; 4-amino-5-(4-bromothiophene-2-yl)-7-(4-methoxyphenyl)pyrido[2,3-d]pyrimidine; 4-amino-5-(3-bromophenyl)methyl-7-(4-(dimethylamino)phenyl)pyrido[2,3-d]pyrimidine; 4-amino-5-(2-phenylethyl)-7-(4-diethylaminophenyl)pyrido[2,3-d]pyrimidine; 4-amino-5-(2-methylpropyl)-7-(4-diethylaminophenyl)pyrido[2,3-d]pyrimidine; 4-amino-5-(butyl)-7-(4-diethylaminophenyl)pyrido[2,3-d]pyrimidine; 4-amino-5-(2-(4-bromophenyl)ethyl)-7-(4-diethylaminophenyl)pyrido[2,3-d]pyrimidine; 4-amino-5-(butyl)-7-(4-dimethylaminophenyl)pyrido[2,3-d]pyrimidine; 4-amino-5-(2-(3-cyanophenyl)methyl)-7-(4-dimethylaminophenyl)pyrido[2,3-d]pyrimidine; 4-amino-5-(2-(N-phenylmethoxycarbonyl)aminoethyl)-7-(4-dimethylaminophenyl)pyrido[2,3-d]pyrimidine; 4-amino-5-(cycloheptyl)-7-(4-dimethylaminophenyl)pyrido[2,3-d]pyrimidine; 4-amino-5-(2-(5-chloro-2-(thiophen-3-yl)phenylmethyl)-7-(4-dimethylaminophenyl)pyrido[2,3-d]pyrimidine; 4-amino-5-(pentyl)-7-(4-diethylaminophenyl)pyrido[2,3-d]pyrimidine; 4-amino-5-hexyl-7-(4-diethylaminophenyl)pyrido[2,3-d]pyrimidine; 4-amino-5-(2-(3-bromophenyl)ethyl)-7-(4-diethylaminophenyl)pyrido[2,3-d]pyrimidine; 4-amino-5-((2-bromophenyl)methyl)-7-(4-diethylaminophenyl)pyrido[2,3-d]pyrimidine; 4-amino-5-cyclopropyl-7-(4-dimethylaminophenyl)pyrido[2,3-d]pyrimidine; 4-amino-5-cyclohexyl-7-(4-dimethylaminophenyl)pyrido[2,3-d]pyrilTudine; 4-amino-5-((2-bromo-5-chlorophenyl)methyl)-7-(4-diethylaminophenyl)pyrido[2,3-d]pyrimidine; 4-amino-5-methyl-7-(4-diethylaminophenyl)pyrido[2,3-d]pyrimidine; 4-amino-5-(2,3-methylenedioxyphenyl)-7-(4-dimethylaminophenyl)pyrido[2,3-d]pyrimidine; 4-amino-5-(3-fluoro-5-trifluoromethylphenyl)-7-(4-dimethylaminophenyl)pyrido[2,3-d]pyrimidine; 4-amino-5-(2-bromophenyl)-7-(4-dimethylaminophenyl)pyrido[2,3-d]pyrimidine; 4-amino-5-(3,5-dimethylphenyl)-7-(4-dimethylaminophenyl)pyrido[2,3-d]pyrimidine; 4-amino-5-(3,4-dichlorophenyl)-7-(4-dimethylaminophenyl)pyrido[2,3-d]pyrimidine; 4-amino-5-(4-fluoro-3-trifluoromethylphenyl)-7-(4-dimethylaminophenyl)pyrido[2,3-d]pyrimidine; 4-amino-5-(3-bromo-5-methoxyphenyl)-7-(4-morpholinylphenyl)pyrido[2,3-d]pyrimidine; 4-amino-5-(3-bromo-5-methoxyphenyl)-7-(4-pyrrolidinylphenyl)pyrido[2,3-d]pyrimidine; 4-amino-5-(3-bromo-5-methoxyphenyl)-7-(4-piperidinylphenyl)pyrido[2,3-d]pyrimidine; 4-amino-5-(3-bromo-5-methoxyphenyl)-7-(4-dimethylaminophenyl)pyrido[2,3-d]pyrimidine; 4-amino-5-(3-methylthiophenyl)-7-(4-dimethylaminophenyl)pyrido[2,3-d]pyrimidine; 4-amino-5-(3-bromo-5-methoxyphenyl)-7-(thiophene-2-yl)pyrido[2,3-d]pyrimidine; 4-amino-5-(2,3-dimethoxyphenyl)-7-(4-dimethylaminophenyl)pyrido[2,3-d]pyrimidine; 4-amino-5-(3-methylsulfonylphenyl)-7-(4-dimethylaminophenyl)pyrido[2,3-d]pyrimidine; 4-acetylamino-5-(3-bromophenyl)-7-(4-dimethylaminophenyl)pyrido[2,3-d]pyrimidine; 4-formylamino-5-(3-bromophenyl)-7-(4-dimethylaminophenyl)pyrido[2,3-d]pyrimidine; 4-(methoxyacetyl)amino-5-(3-bromophenyl)-7-(4-diethylaminophenyl)pyrido[2,3-d]pyrimidine; 4-trifluoroacetylamino-5-(3-bromophenyl)-7-(4-dimethylaminophenyl)pyrido[2,3-d]pyrimidine; 4-pentanoylamino-5-(3-bromophenyl)-7-(4-dimethylaminophenyl)pyrido[2,3-d]pyrimidine; 4-benzoylamino-5-(3-bromophenyl)-7-(4-dimethylaminophenyl)pyrido[2,3-d]pyrimidine; 4-(N—BOC-glycyl)amino-5-(3-bromophenyl)-7-(4-dimethylaminophenyl)pyrido[2,3-d]pyrimidine; 4-(N-phthalimidylglycyl)amino-5-(3-bromophenyl)-7-(4-dimethylaminophenyl)pyrido[2,3-d]pyrimidine; 4-(ethoxycarbonyl)amino-5-(3-bromophenyl)-7-(4-dimethylaminophenyl)pyrido[2,3-d]pyrimidine; 4-(ethylaminocarbonyl)amino-5-(3-bromophenyl)-7-(4-dimethylaminophenyl)pyrido[2,3-d]pyrimidine; 4-allylamino-5-(3-bromophenyl)-7-(4-dimethylaminophenyl)pyrido[2,3-d]pyrimidine; 4-(2-(N,N-dimethylamino)ethylamino)-5-(4-bromophenyl)-7-(4-dimethylaminophenyl)pyrido[2,3-d]pyrimidine; 4-(4-(N,N-dimethylamino)butylamino)-5-(3-bromophenyl)-7-(4-dimethylaminophenyl)pyrido[2,3-d]pyrimidine; 4-(N-allyl-N-formylamino)-5-(4-dimethylaminophenyl)-7-(4-bromophenyl)pyrido[2,3-d]pyrimidine; 4-diacetylamino-5-(p-dimethylaminophenyl)-7-(4-bromophenyl)pyrido[2,3-d]pyrimidine; 4-amino-5-(3-bromophenyl)-7-(5-amino-2-pyridyl)pyrido[2,3-d]pyrimidine; 4-amino-5-(3-bromophenyl)-7-(5-dimethylamino-2-pyridyl)pyrido[2,3-d]pyrimidine; 4-amino-5-(3-bromophenyl)-7-(5-dimethylamino-2-pyrazinyl)pyrido[2,3-d]pyrimidine; 4-amino-5-(3-bromophenyl)-7-(2-oxobenzoxazolin-6-yl)pyrido[2,3-d]pyrimidine; 4-amino-5-(3-bromophenyl)-7-(1-methyl-2-oxobenzoxazolin-6-yl)pyrido[2,3-d]pyrimidine; 4-amino-5-((5-chloro-2-(3-methoxyphenyl)phenyl)methyl)-7-(4-dimethylaminophenyl)pyrido[2,3-d]pyrimidine; 4-amino-5-((thiophene-2-yl)methyl)-7-(4-diethylaminophenyl)pyrido[2,3-d]pyrimidine; 4-amino-5-((thiophene-3-yl)methyl)-7-(4-diethylaminophenyl)pyrido[2,3-d]pyrimidine; 4-amino-5-((2-bromophenyl)methyl)-7-(4-dimethylaminophenyl)pyrido[2,3-d]pyrimidine; 4-amino-5-(3-bromophenyl)-7-(4-(N-formyl-N-(2-methoxyethyl)amino)phenyl)pyrido[2,3-d]pyrimidine; 4-amino-5-(3-bromophenyl)-7-(4-(N-(2-methoxyethyl)amino)phenyl)pyrido[2,3-d]pyrimidine; 4-amino-5-(3-bromophenyl)-7-(4-(N-methyl-N-((2-dimethylamino)ethyl)amino)phenyl)pyrido[2,3-d]pyrimidine; 4-amino-5-(3-bromophenyl)-7-(4-(2-methoxy)acetylamino)ethyl)amino)phenyl)pyrido[2,3-d]pyrimidine; 4-amino-5-(3-bromophenyl)-7-((4-formylamino)phenyl)pyrido[2,3-d]pyrimidine; 4-amino-5-(3-bromophenyl)-7-(4-(2-(dimethylamino)acetylamino)phenyl)pyrido[2,3-d]pyrimidine; 4-amino-5-(3-bromophenyl)-7-(4-(2-oxo-3-oxazolidinyl)phenyl)pyrido[2,3-d]pyrimidine; 4-amino-5-(3-bromophenyl)-7-(6-(2-propyl)-3-pyridinyl)pyrido[2,3-d]pyrimidine; 4-amino-5-(3-bromophenyl)-7-(3-methyl-4-pyrrolidinylphenyl)pyrido[2,3-d]pyrimidine; 4-amino-5-(3-bromophenyl)-7-(6-imidazolyl-3-pyridinyl)pyrido[2,3-d]pyrimidine; 4-amino-5-phenylmethyl-7-(4-diethylaminophenyl)pyrido[2,3-d]pyrimidine; 4-amino-5-(2-(3-aminopropynyl)phenylmethyl)-7-(4-diethylaminophenyl)pyrido[2,3-d]pyrimidine; 4-amino-5-(1-(3-bromophenyl)ethyl)-7-(4-diethylaminophenyl)pyrido[2,3d]pyrimidine; 4-amino-5-(4-dimethylaminophenyl)-7-(4-bromophenyl)pyrido[2,3-d]pyrimidine; 4-amino-5-(2-furanyl)-7-(4-(N-morpholinyl)phenyl)pyrido[2,3-d]pyrimidine; 4-amino-5-(3-bromophenyl)-7-(2-dimethylamino-5-pyrimidinyl)pyrido[2,3-d]pyrimidine; 4-amino-5-(3-bromophenyl)-7-(4-(ureido)phenyl)pyrido[2,3-d]pyrimidine; 4-amino-5-(1-phenylmethyl-3-piperidinyl)-7-(4-diethylaminophenyl)pyrido[2,3-d]pyrimidine; 4-amino-5-(3-bromophenyl)-7-(6-(3-methyl-5-isoxazolyl))-3-pyridinyl)pyrido[2,3-d]pyrimidine; 4-amino-5-(3-bromophenyl)-7-(6-chloro-3-pyridinyl)pyrido[2,3-d]pyrimidine; 4-amino-5-(3-bromophenyl)-7-(6-methoxy-3-pyridinyl)pyrido[2,3-d]pyrimidine; 4-amino-5-(3-bromophenyl)-7-(6-(1,2,4-triazol-4-yl)-3-pyridinyl)pyrido[2,3-d]pyrimidine; 4-amino-5-(3-bromophenyl)-7-(2-morpholinyl-5-pyrimidinyl)pyrido[2,3-d]pyrimidine; 4-amino-5-(2-thiazolyl)-7-(4-pyrrolidinylphenyl)-pyrido[2,3-d]pyrimidine; 4-amino-5-(3-bromophenyl)-7-(6-pyrazolyl-3-pyridinyl))-pyrido[2,3-d]pyrimidine; 4-amino-5-(3-bromophenyl)-7-(4-(1-methyl-ureido)phenyl)-pyrido[2,3-d]pyrimidine; 4-amino-5-(3-bromophenyl)-7-(4-(N-methyl-N-(2-pyrimidinyl)amino)phenyl)-pyrido[2,3-d]pyrimidine; 4-amino-5-(3-bromophenyl)-7-(3-fluoro-4-(N-formyl-N-methylamino)phenyl)-pyrido[2,3-d]pyrimidine; 4-formylamino-S-(3-bromophenyl)-7-(3-fluoro-4-(N-formyl-N-methylamino)phenyl)-pyrido[2,3-d]pyrimidine; 4-amino-5-(3-bromophenyl)-7-(4-(N-methyl-N-methylsulfonylamino)-phenyl)pyrido[2,3-d]pyrimidine; 4-amino-5-(3-bromophenyl)-7-(6-(N-methyl-N-methylSulfonylamino)-3-pyridinyl)pyrido[2,3-d]pyrimidine; 4-amino-5-(3-bromophenyl)-7-(1-methyl-5-indolinyl)pyrido[2,3-d]pyrimidine; 4-amino-5-(3-bromophenyl)-7-(1-methyl-5-benzimidazolyl)pyrido[2,3-d]pyrimidine; 4-amino-5-(3-bromophenyl)-7-(6-dimethylamino-3-pyridazinyl)pyrido[2,3-d]pyrimidine; 4-amino-5-(3bromophenyl)-7-(6-morpholinyl-3-pyridazinyl)pyrido[2,3-d]pyrimidine; 4-amino-5-(3-bromophenyl)-7-(6-pyrrolidinyl-3-pyridazinyl)pyrido[2,3-d]pyrimidine; 4-amino-5-(3-bromophenyl)-7-(5-morpholinyl-2-pyrazinyl)pyrido[2,3-d]pyrimidine; 4-amino-5-(3-bromophenyl)-7-(5-(N-(2-methoxyethyl)-N-methylamino)-2-pyrazinyl)pyrido[2,3-d]pyrimidine; 4-amino-5-(3-bromophenyl)-7-(4-(morpholinylmethyl)-phenyl)pyrido[2,3-d]pyrimidine; 4-amino-5-(3-bromophenyl)-7-(5-(N,N-bis(2-methoxyethyl)amino)-2-pyridinyl)pyrido[2,3-d]pyrimidine; 4-amino-5-(3-bromophenyl)-7-(4-(imidazolylmethyl)-phenyl)pyrido[2,3-d]pyrimidine; 4-amino-5-(3-bromophenyl)-7-(5-(1-morpholinyl)-2-pyridinyl)pyrido[2,3-d]pyrimidine; 4-amino-5-(3-bromophenyl)-7-(4-((dimethylamino)methyl)-phenyl)pyrido[2,3-d]pyrimidine; 4-amino-5-(3-bromophenyl)-7-(5-(4-hydroxy-1-piperidinyl)-2-pyridinyl)pyrido[2,3-d]pyrimidine; 4-amino-5-(3-bromophenyl)-7-(5-(N-formyl-N-methylamino)-2-pyridinyl)pyrido[2,3-d]pyrimidine; 4-amino-5-(3-bromophenyl)-7-(5-(2-propenyl)-2-pyridinyl)pyrido[2,3-d]pyrimidine; 4-amino-5-(3-bromophenyl)-7-(3-(2-methoxyethyl)-2-oxo-6-benzoxazolyl)pyrido[2,3-d]pyrimidine; 4-amino-5-(3-bromophenyl)-7-(4-(1-(N-formylamino)-ethyl)phenyl)pyrido[2,3-d]pyrimidine; 4-(methylamino)-5-(3-bromophenyl)-7-(4-dimethylaminophenyl)pyrido[2,3-d]pyrimidine hydrochloride; 4-(2-methoxyethylamino)-5-(3-bromophenyl)-7-(4-dimethylaminophenyl)pyrido[2,3-d]pyrimidine hydrochloride; 4-amino-5-(3-bromophenyl)-7-(4-(1-methyl-2-imidazolyl)phenyl)pyrido[2,3-d]pyrimidine trihydrochloride; 4-amino-5-(3-bromophenyl)-7-(4-(aminomethyl)phenyl)pyrido[2,3-d]pyrimidine; 4-amino-5-(3-bromophenyl)-7-(2-bromo-4-(dimethylamino)phenyl)pyrido[2,3-d]pyrimidine; 4-amino-5-(3-bromophenyl)-7-(4-(dimethylaminoethyl)phenyl)pyrido[2,3-d]pyrimidine; 4-amino-5-(3-bromophenyl)-7-(4-(3-(dimethylamino)propynyl)phenyl)pyrido[2,3-d]pyrimidine; 4-amino-5-(3-bromophenyl)-7-(4-(3-amino-3-methylbutynyl)phenyl)pyrido[2,3-d]pyrimidine; 4-amino-5-(3-bromophenyl)-7-(4-dimethylphosphonatophenyl)pyrido[2,3-d]pyrimidine; 4-amino-5-(3-bromophenyl)-7-(4-(3-(methoxypropynyl)pyrido[2,3-d]pyrimidine; 4-amino-5-(3-bromophenyl)-7-(4-carboxyphenyl)pyrido[2,3-d]pyrimidine; 4-amino-5-(3-bromophenyl)-7-(4-methyl-3-oxo-2H-4H-pyrido[3,2-b]-1,4-oxazin-7-yl)pyrido[2,3-d]pyrimidine; 4-amino-5-(3-bromophenyl)-7-(4-(2-dimethylamino)ethyl)-3-oxo-2H-4H-pyrido[3,2-b]-1,4-oxazin-7-yl)pyrido[2,3-d]pyrimidine; 4-amino-5-(3-bromophenyl)-7-(2,3-dihydro-3-(dimethylaminoethyl)-2-oxobenzoxazol-6-yl)pyrido[2,3-d]pyrimidine; 4-amino-5-(3-bromophenyl)-7-(4-methyl-3-oxo-2H-4H-benzo-1,4-oxazin-7-yl)pyrido[2,3-d]pyrimidine; 4-amino-5-(3-bromophenyl)-7-(2,2,4-trimethyl-3-oxo-2H-4H-benzo-1,4-oxazin-7-yl)pyrido[2,3-d]pyrimidine; 4-amino-5-cyclohexyl-7-(4-(2-dimethylamino)ethyl)-2H-4H-benzo-3-oxo-1,4-oxazin-7-yl)pyrido[2,3-d]pyrimidine; 4-amino-5-(3-bromophenyl)-7-(5-(1-methylethyl)-2-pyridyl)pyrido[2,3-d]pyrimidine; 4-amino-5-(3-bromophenyl)-7-(5-piperidin-1-ylpyrid-2-yl)pyrido[2,3-d]pyrimidine; 4-amino-5-(1-(4-bromophenyl)ethyl)-7-(6-morpholinylpyrid-3-yl)pyrido[2,3-d]pyrimidine; 4-amino-5-(3-bromophenyl)-7-(4-((N-formylamino)methyl)phenyl)pyrido[2,3-d]pyrimidine; 4-amino-5-(3-bromophenyl)-7-(4-(1-methyl-1-(N-methylamino)ethyl)phenyl)-pyrido[2,3-d]pyrimidine; 4-amino-5-(3-bromophenyl)-7-(4-(1-(dimethylamino)-1-methylethyl)phenyl)pyrido[2,3-d]pyrimidine; 4-amino-5-(3-bromophenyl)-7-(N-acetyl-5-indolinyl)pyrido[2,3-d]pyrimidine; 4-amino-5-cyclohexyl-7-(6-chloro-3-pyridyl)pyrido[2,3-d]pyrimidine; 4-amino-5-(1-(2-bromophenyl)ethyl)-7-(6-diethylamino-3-pyridyl)pyrido[2,3-d]pyrimidine; 4-amino-5-(1-(2-bromophenyl)ethyl)-7-(6-morpholinyl-3-pyridyl)pyrido[2,3-d]pyrimidine; 4-amino-5-(1-(2-bromophenyl)ethyl)-7-(4-(N-methyl-N-formyl)amino)-phenyl)pyrido[2,3-d]pyrimidine; 4-amino-5-cyclohexyl-7-(6-morpholinyl-3-pyridyl)pyrido[2,3-d]pyrimidine; 4-amino-5-((2-bromophenyl)methyl)-7-(6-morpholinyl-3-pyridyl)pyrido[2,3-d]pyrimidine; 4-amino-S-(4-tetrahydropyranyl)-7-(6-morpholinyl-3-pyridyl)pyrido[2,3-d]pyrimidine; 4-amino-5-cyclohexyl-7-(6-dimethylamino-3-pyridyl)pyrido[2,3-d]pyrimidine; 4-amino-5-(1-ethylpropyl)-7-(6-dimethylamino-3-pyridyl)pyrido[2,3-d]pyrimidine; 4-amino-5-cyclopentyl-7-(6-morpholinyl-3-pyridyl)pyrido[2,3-d]pyrimidine; 4-amino-5-cyclohexyl-7-(2-chloro-3-pyridyl)pyrido[2,3-d]pyrimidine; 4-amino-5-(3,5-dimethylcyclohexyl)-7-(6-dimethylamino-3-pyridyl)pyrido[2,3-d]pyrimidine; 4-amino-5-((N-(benzyloxycarbonyl)-4-piperidinyl)methyl)-7-(6-morpholinyl-3-pyridyl)pyrido[2,3-d]pyrimidine; 4-amino-5-cyclohexyl-7-(6-bromo-3-pyridyl)pyrido[2,3-d]pyrimidine; 4-amino-5-cyclohexyl-7-(3-cyanophenyl)pyrido[2,3-d]pyrimidine; 4-amino-S-(1-(2-bromophenyl)ethyl)-7-(6-dimethylamino-3-pyridazinyl)pyrido[2,3-d]pyrimidine; 4-amino-5-(3-bromophenyl)-7-(6-imidazolyl-3-pyridazinyl)pyrido[2,3-d]pyrimidine; 4-amino-5-(3-bromophenyl)-7-(6-(azacycloheptanyl)-3-pyridazinyl)pyrido[2,3-d]pyrimidine; 4-amino-5-(3-bromophenyl)-7-(6-(N-methyl-N-(1-methylethyl)amino)-3-pyridazinyl)pyrido[2,3-d]pyrimidine; 4-amino-5-(1-(2-bromophenyl)ethyl)-7-(6-morpholinyl-3-pyridazinyl)pyrido[2,3-d]pyrimidine; 4-amino-5-cyclohexyl-7-(6-(4-acetylpiperazinyl)-3-pyridyl)pyrido[2,3-d]pyrimidine; 4-amino-5-cyclohexyl-7-(6-(4-acetyl-1,4-diazacycloheptanyl)-3-pyridyl)pyrido[2,3-d]pyrimidine; 4-amino-S-cyclohexyl-7-(6-(4-methyl-1,4-diazacycloheptanyl)-3-pyridyl)pyrido[2,3-d]pyrimidine; 4-amino-5-cyclohexyl-7-(6-(N-methyl-N-(2-(2-pyridyl)ethyl)amino)-3-pyridyl)pyrido[2,3-d]pyrimidine; 4-amino-5-cyclohexyl-7-(6-2-(N—(N′,N′-dimethylaminoethyl)-N-methylamino)-3-pyridyl)pyrido[2,3-d]pyrimidine; 4-amino-5-cyclohexyl-7-(6-azetidinyl-3-pyridyl)pyrido[2,3-d]pyrimidine; 4-amino-5-cyclohexyl-7-(6-(3-(N-methylacetamido)pyrrolidinyl)pyridyl)pyrido[2,3d]pyrimidine; 4-amino-5-cyclohexyl-7-(6-(3-(formamido)pyrrolidinyl)pyridyl)pyrido[2,3-d]pyrimidine; 4-amino-5-cyclohexyl-7-(4-oxo-1-phenyl-1,3,8-triazaspiro[4,5[decan-8-yl)pyrido[2,3-d]pyrimidine; 4-amino-5-cyclohexyl-7-(6-(2-methoxymethyl)pyrrolidin-1-yl)pyridyl)pyrido[2,3-d]pyrimidine; 4-amino-5-cyclohexyl-7-(6-(N-methoxyethyl-N-propylamino)pyridyl)pyrido[2,3-d]pyrimidine; 4-amino-5-cyclohexyl-7-(N-methyl-N-(2,2-dimethoxyethyl)amino)pyrido[2,3-d]pyrimidine; 4-amino-5-cyclohexyl-7-(6-(4-(dimethylamino)piperidinyl)pyridyl)pyrido[2,3-d]pyrimidine; 4-amino-5-cyclohexyl-7-(6-(4-(aminocarbonyl)piperidinyl)pyridyl)pyrido[2,3-d]pyrimidine; 4-amino-5-cyclohexyl-7-(N-methyl-N-(3-(diethylamino)propyl)aminopyrid-3-yl)pyrido[2,3-d]pyrimidine; 4-amino-5-cyclohexyl-7-(6-(N-methyl-N-(4-pyridyl)ethylamino)pyrid-3-yl)pyrido[2,3-d]pyrimidine; 4-amino-5-cyclohexyl-7-(6-(N-methyl-N-(3-pyridylmethylamino)pyrid-3-yl)pyrido[2,3-d]pyrimidine; 4-amino-5-(1-(2-bromophenyl)ethyl)-7-(1-methyl-5-indolyl)pyrido[2,3-d]pyrimidine; 4-amino-5-(1-(2-bromophenyl)ethyl)-7-(1-methyl-2,3-dioxo-5-indolyl)pyrido[2,3-d]pyrimidine; 4-amino-5-(3-bromophenyl)-7-(3-fluoro-4-(1-morpholinyl)phenyl)pyrido[2,3-d]pyrimidine; 4-amino-5-(3-bromophenyl)-7-(4-hydroxy-3-nitrophenyl)pyrido[2,3-d]pyrimidine; 4-amino-5-(3-bromophenyl)-7-(6-(4,4-ethylenedioxypiperidinyl)-3-pyridyl)pyrido[2,3-d]pyrimidine; 4-amino-5-(3-bromophenyl)-7-(6-(4-oxopiperidinyl)-3-pyridyl)pyrido[2,3-d]pyrimidine; 4-amino-5-(3-bromophenyl)-7-(6-(4-formylpiperazinyl)-3-pyridyl)pyrido[2,3d]pyrimidine; 4-amino-5-(3-bromophenyl)-7-(6-(4-methylpiperazinyl)-3-pyridyl)pyrido[2,3-d]pyrimidine; 4-amino-5-(3-bromophenyl)-7-(6-morpholinyl)-3-pyridyl)pyrido[2,3-d]pyrimidin; 4-amino-5-(3-bromophenyl)-7-(6-(4,4-dioxothiomorpholinyl)-3-pyridyl)pyrido[2,3-d]pyrimidine; 4-amino-5-(2-bromophenyl)-7-(6-morpholinyl-3-pyridyl)pyrido[2,3-d]pyrimidine; 4-amino-5-(3-bromo-4-methoxyphenyl)-7-(6-morpholinyl-3-pyridyl)pyrido[2,3-d]pyrimidine; 4-amino-5-(4-bromophenyl)-7-(6-morpholinyl-3-pyridyl)pyrido[2,3-d]pyrimidine; 4-amino-5-(3-chlorophenyl)-7-(6-morpholinyl-3-pyridyl)pyrido[2,3-d]pyrimidine; 4-amino-5-(3-bromophenyl)-7-(5-chloro-6-morpholinyl-3-pyridyl)pyrido[2,3-d]pyrimidine; 4-amino-5-(3-bromophenyl)-7-(6-(N-oxidomorpholinyl)-3-pyridyl)pyrido[2,3-d]pyrimidine; 4-amino-5-(3-bromophenyl)-7-(6-(N-(2-hydroxyethoxyethyl)amino)-3-pyridyl)pyrido[2,3-d]pyrimidine; 4-amino-5-(3-bromophenyl)-7-(6-(N-(2-hydroxyethoxyethyl)-N-formylamino)-3-pyridyl)pyrido[2,3-d]pyrimidine; 4-amino-5-(3-bromophenyl)-7-(6-(N-(2-hydroxyethoxyethyl)-3-pyridyl-N-oxide)pyrido[2,3-d]pyrimidine; 4-amino-5-(3-bromophenyl)-7-(6-(3-hydroxy)morpholinyl)-3-pyridyl)pyrido[2,3-d]pyrimidine; 1-(5-(4-amino-5-(3-bromophenyl)pyrido[2,3-d]pyrimidin-7-yl)-2-pyridyl)-piperidine-4-phosphate, disodium salt; 4-amino-5-(3-bromophenyl)-7-(4-methylenylpiperidinyl)-3-pyridyl)pyrido[2,3-d]pyrimidine; 4-amino-5-(3-bromophenyl)-7-(4-hydroxy-4-(hydroxymethyl)piperidinyl)-3-pyridyl)pyrido[2,3-d]pyrimidine; 4-amino-5-(3-bromophenyl)-7-(6-(4,4-ethylenedioxypiperidinyl)-3-pyridyl)pyrido[2,3-d]pyrimidine; 4-amino-5-cyclohexyl-7-(6-(4-oxo-piperidinyl)-3-pyridyl)pyrido[2,3-d]pyrimidine; 4-amino-5-cyclohexyl-7-(6-(4-methylenylpiperidinyl)-3-pyridyl)pyrido[2,3-d]pyrimidine; 4-N-(iminomethyl)amino-5-cyclohexyl-7-(6-dimethylamino-3-pyridyl)pyrido[2,3-d]pyrimidine; 4-amino-5-(4-dimethylaminophenyl)-7-(4-bromophenyl)pyrido[2,3-d]pyrimidine; 4-amino-S-(4-dimethylaminophenyl)-7-(4-dimethylaminophenyl)pyrido[2,3-d]pyrimidine; 4-amino-S-(4-methoxyphenyl)-7-(4-dimethylaminophenyl)pyrido[2,3-d]pyrimidine; 4-amino-S-(4-dimethylaminophenyl)-7-(4-methoxyphenyl)pyrido[2,3-d]pyrimidine; 4-amino-5-(4-(2-propyl)phenyl)-7-(4-methoxyphenyl)pyrido[2,3-d]pyrimidine; 4-amino-5-(4-neopentylphenyl)-7-(4-methoxyphenyl)pyrido[2,3-d]pyrimidine; 4-amino-5-(4-butyloxyphenyl)-7-(4-methoxyphenyl)pyrido[2,3-d]pyrimidine; 4-amino-5-(4-methoxyphenyl)-7-(4-bromophenyl)pyrido[2,3-d]pyrimidine; 4-amino-5-(4-(2-propyl)oxyphenyl)-7-(4-methoxyphenyl)pyrido[2,3-d]pyrimidine; 4-amino-5-(4-butoxyphenyl)-7-(4-N-formylpiperazinylphenyl)pyrido[2,3-d]-pyrimidine; 4-amino-5-(4-benzyloxyphenyl)-7-(4-methoxyphenyl)pyrido[2,3-d]pyrimidine; 4-amino-5-(4-phenoxyphenyl)-7-(4-methoxyphenyl)pyrido[2,3-d]pyrimidine; 4-amino-5-(4-(2-propyl)phenyl)-7-(4-diethylmalonylallylphenyl)pyrido[2,3-d]pyrimidine; 4-amino-5-(4-(2-propyl)phenyl)-7-(4-t-butylacrylphenyl)pyrido[2,3-d]pyrimidine; 4-amino-5-(3-bromophenyl)-7-(4-dimethylaminophenyl)pyrido[2,3-d]pyrimidine; 4-amino-5-(3,4-dimethoxyphenyl)-7-(4-dimethylaminophenyl)pyrido[2,3-d]pyrimidine; 4-amino-5-(3-t-butylacrylphenyl)-7-(4-dimethylaminophenyl)pyrido[2,3-d]pyrimidine; 4-amino-5-(3-methoxyphenyl-7-(4-dimethylaminophenyl)pyrido[2,3-d]pyrimidine; 4-amino-5-(3,5-dimethoxyphenyl-7-(4-dimethylaminophenyl)pyrido[2,3-d]pyrimidine; 4-amino-5-(3-diethylmalonylallylphenyl)-7-(4-dimethylaminophenyl)pyrido[2,3-d]pyrimidine; 4-amino-5-(3-vinylpyridinylphenyl)-7-(4-dimethylaminophenyl)pyrido[2,3-d]pyrimidine; 4-amino-5-(3-trifluoromethylphenyl)-7-(4-dimethylaminophenyl)pyrido[2,3-d]pyrimidine; 4-amino-5-(3 carboxamidophenyl)-7-(4-dimethylaminophenyl)pyrido[2,3-d]pyrimidine; 4-amino-5-(3-cyanophenyl)-7-(4-dimethylaminophenyl)pyrido[2,3-d]pyrimidine; 4-amino-5-(3-benzyloxyphenyl)-7-(4-dimethylaminophenyl)pyrido[2,3-d]pyrimidine; 4-amino-5-(3-methoxyphenyl)-7-(4-methoxyphenyl)pyrido[2,3-d]pyrimidine; 4-amino-5-(3-bromophenyl)-7-(4-butoxyphenyl)pyrido[2,3-d]pyrimidine; 4-amino-5-(3-(2-pyridyl)phenyl)-7-(4-dimethylaminophenyl)pyrido[2,3-d]pyrimidine; 4-amino-5-(3-methylphenyl)-7-(4-dimethylaminophenyl)pyrido[2,3-d]pyrimidine; 4-amino-5-(3-chlorophenyl)-7-(4-dimethylaminophenyl)pyrido[2,3-d]pyrimidine; 4-amino-S-(3-fluorophenyl)-7-(4-dimethylaminophenyl)pyrido[2,3-d]pyrimidine; 4-amino-5-(3-bromophenyl)-7-(4-methoxyphenyl)pyrido[2,3-d]pyrimidine; 4-amino-5-(3-methoxyphenyl)-7-(4-bromophenyl)pyrido[2,3-d]pyrimidine; 4-amino-5-(3-bromophenyl)-7-phenylpyrido[2,3-d]pyrimidine; 4-amino-5-(3-bromophenyl)-7-(4-ethylphenyl)pyrido[2,3-d]pyrimidine; 4-amino-5-(3-bromophenyl)-7-(4-bromophenyl)pyrido[2,3-d]pyrimidine; 4-amino-5-(3-bromophenyl)-7-(4-cyanophenyl)pyrido[2,3-d]pyrimidine; 4-amino-5-(3-bromophenyl)-7-(4-hydroxyphenyl)pyrido[2,3-d]pyrimidine; 4-amino-5-(3-iodophenyl)-7-(4-dimethylaminophenyl)pyrido[2,3-d]pyrimidine; 4-amino-5-(3-ethoxyphenyl)-7-(4-dimethylaminophenyl)pyrido[2,3-d]pyrimidine; 4-amino-5-(3-trifloromethyoxyphenyl)-7-(4-dimethylaminophenyl)pyrido[2,3-d]pyrimidine; 4-amino-5-(3,5-dichlorophenyl)-7-(4-dimethylaminophenyl)pyrido[2,3-d]pyrimidine; 4-amino-5-(3-bromo-4-fluorophenyl)-7-(4-dimethylaminophenyl)pyrido[2,3-d]pyrimidine; 4-amino-5-(3-hydroxyphenyl)-7-(4-dimethylaminophenyl)pyrido[2,3-d]pyrimidine; 4-amino-5-(3-bromophenyl)-7-(4-morpholinylphenyl)pyrido[2,3-d]pyrimidine; 4-amino-5-(3-bromophenyl)-7-(4-piperidinylphenyl)pyrido[2,3-d]pyrimidine; 4-amino-5-(3-bromophenyl)-7-(4-(imidazol-1-yl)phenyl)pyrido[2,3-d]pyrimidine; 4-amino-5-(3-bromophenyl)-7-(4-chlorophenyl)pyrido[2,3-d]pyrimidine; 4-amino-5-(3-bromophenyl)-7-(4-isopropylphenyl)pyrido[2,3-d]pyrimidine; 4-amino-5-(3-bromophenyl)-7-(4-trifluorophenyl)pyrido[2,3-d]pyrimidine; 4-amino-5-(3-bromophenyl)-7-(4-diethylaminophenyl)pyrido[2,3-d]pyrimidine; 4-amino-5-(3-bromophenyl)-7-(3,4,5-trimethoxyphenyl)pyrido[2,3-d]pyrimidine; 4-amino-5-(3-(3-methoxybenzyl)phenyl)-7-(4-dimethylaminophenyl)pyrido[2,3-d]pyrimidine; samino-5-(3-methoxyethyoxyphenyl)-7-(4-dimethylaminophenyl)pyrido[2,3-d]pyrimidine; 4-amino-5-(3,4-methylenedioxyphenyl)-7-(4-dimethylaminophenyl)pyrido[2,3-d]pyrimidine; 4-amino-5-(3-bromophenyl)-7-(4-ethoxyphenyl)pyrido[2,3-d]pyrimidine; 4-amino-5-(3-bromophenyl)-7-(2′-thiophene)pyrido[2,3-d]pyrimidine; 4-amino-5-(3-bromophenyl)-7-(4-fluorophenyl)pyrido[2,3-d]pyrimidine; 4-amino-5-(3-dimethylaminophenyl)-7-(4-dimethylaminophenyl)pyrido[2,3-d]pyrimidine; 4-amine-5-phenyl-7-(4-dimethylaminophenyl)pyrido[2,3-d]pyrimidine; 4-amino-5-(3,4,5-trimethoxyphenyl)-7-(4-dimethylaminophenyl)pyrido[2,3-d]pyrimidine; 4-amino-5-(3-bromophenyl)-7-(4-nitrophenyl)pyrido[2,3-d]pyrimidine; 4-amino-5-(3-bromophenyl)-7-(4-iodophenyl)pyrido[2,3-d]pyrimidine; 4-amino-5-(3-bromophenyl)-7-amino-S-(3-bromophenyl)-7-(3,4-methylenedioxyphenyl)pyrido[2,3-d]pyrimidine; 4-amino-5-(thiophen-2-yl)-7-(4-morpholinylphenyl)pyrido[2,3-d]pyrimidine; 4-amino S-(3,S-dimethoxyphenyl)-7-(thiophen-2-yl)pyrido[2,3-d]pyrimidine; 4-amino-5-(3-bromophenyl)-7-(4-carboxamidophenyl)pyrido[2,3-d]pyrimidine; 4-amino-5-(3-bromophenyl)-7-(4-(2-methoxy)ethoxyphenyl)pyrido[2,3-d]pyrimidine; 4-amino-5-(3,5-dimethoxyphenyl)-7-(4-morpholinylphenyl)pyrido[2,3-d]pyrimidine; 4-amino-5-(3 trifluoromethylphenyl)-7-(thiophene-2-yl)pyrido[2,3-d]pyrimidine; 4-amino-5-(3-bromophenyl)-7-(4-aminophenyl)pyrido[2,3-d]pyrimidine; 4-amino-5-(3-bromo-4-fluorophenyl)-7-(thiophene-2-yl)pyrido[2,3-d]pyrimidine; 4-amino-5-(3-bromo-4-fluorophenyl)-7-(2-furanyl)pyrido[2,3-d]pyrimidine; 4-amino-5-(3,5-dimethoxyphenyl)-7-(4-iodophenyl)pyrido[2,3-d]pyrimidine; 4-amino-5-(3,5-dimethoxyphenyl)-7-(4-imidazolylphenyl)pyrido[2,3-d]pyrimidine; 4-amino-5-(3,5-dimethoxyphenyl)-7-(4-(thiophene-2-yl)phenyl)pyrido[2,3-d]pyrimidine; 4-amino-5-(3,5-dimethoxyphenyl)-7-(4-(3-pyridyl)phenyl)pyrido[2,3-d]pyrimidine; 4-amino-5-(3-bromophenyl)-7-(4-(4-methylpiperidinyl)phenyl)pyrido[2,3-d]pyrimidine; 4-amino-5-(3-bromophenyl)-7-(4-pyrrolidinylphenyl)pyrido[2,3-d]pyrimidine; 4-amino-5-(4-bromothiophene-)-7-(4-dimethylaminophenyl)pyrido[2,3-d]pyrimidine; 4-amino-5-(4-bromothiophene-2-yl)-7-(4-morpholinylphenyl)pyrido[2,3-d]pyrimidine; 4-morpholinyl-5-(3-bromophenyl)-7-(4-dimethylaminophenyl)pyrido[2,3-d]pyrimidine; 4-amino-5-(5-bromothiophene-2-yl)-7-(4-morpholinylphenyl)pyrido[2,3-d]pyrimidine; 4-amino-5-(4-bromophenyl)-7-(4-dimethylaminophenyl)pyrido[2,3-d]pyrimidine; 4-amino-5-(3-bromophenyl)-7-(4-(acetylamino)phenyl)pyrido[2,3-d]pyrimidine; 4-amino-5-(3-bromophenyl)-7-(4-dimethylaminophenyl)pyrido[2,3-d]pyrimidine; 4-amino-5-(3,5-dimethoxyphenyl)-7-(5-pyrimidinylphenyl)pyrido[2,3-d]pyrimidine; 4-(4-fluorophenyl)amino)-5-(3-bromophenyl)-7-(4-dimethylaminophenyl)pyrido[2,3-d]pyrimidine; 4-amino-5-(4-bromothiophene-2-yl)-7-(4-pyrrolidinylphenyl)pyrido[2,3-d]pyrimidine; 4-amino-5-(4-bromothiophene-2-yl)-7-(thiophene-2-yl)pyrido[2,3-d]pyrimidine; 4-amino-5-(3 bromophenyl)-7-(5-(dimethylamino)thiophene-2-yl)pyrido[2,3-d]pyrimidine; 4-amino-5-(3-bromo-5-iodophenyl)-7-(4-(dimethylamino)phenyl)pyrido[2,3-d]pyrimidine; 4-amino-5-(3,5-di(trifluoromethyl)phenyl)-7-(4-(dimethylamino)phenyl)pyrido[2,3-d]pyrimidine; 4-amino-5-(3,5-di(trifluoromethyl)phenyl)-7-(4-morpholinylphenyl)pyrido[2,3-d]pyrimidine; 4-amino-5-(3,5-dibromophenyl)-7-(4-dimethylamino)phenyl)pyrido[2,3-d]pyrimidine; 4-amino-5-(3,5-dibromophenyl)-7-(4-morpholinylphenyl)pyrido[2,3-d]pyrimidine; 4-amino-5-(4-bromothiophene-2-yl)-7-(4-(4-methylpiperidinyl)phenyl)pyrido[2,3-d]pyrimidine; 4-amino-5-(3,5-dibromophenyl)-7-(4-(dimethylamino)phenyl)pyrido[2,3-d]pyrimidine; 4-amino-5-(3-bromophenyl)-7-(3-(dimethylamino)phenyl)pyrido[2,3-d]pyrimidine; 4-amino-5-(3-bromophenyl)-7-(4-methylsulfonylphenyl)pyrido[2,3-d]pyrimidine; 4-amino-5-(3-bromophenyl)-7-(3-methoxyphenyl)pyrido[2,3-d]pyrimidine; 4-amino-5-(3-bromophenyl)-7-(4-(methylthio)phenyl)pyrido[2,3-d]pyrimidine; 4-amino-5-(3-bromophenyl)-7-(3,4-dichlorophenyl)pyrido[2,3-d]pyrimidine; 4-amino-5-(3-bromophenyl)-7-(4-(N-methyl-N-formylamino)phenyl)pyrido[2,3-d]pyrimidine; 4-amino-5-(3-bromophenyl)-7-(4-methylaminophenyl)pyrido[2,3-d]pyrimidine; samino-5-(3-bromo-4-fluorophenyl)-7-(4-methylsulfonylphenyl)pyrido[2,3-d]pyrimidine; 4-amino-5-(3 bromophenyl)-7-(3-amino-4-methoxyphenyl)pyrido[2,3-d]pyrimidine9 4-amino-5-(3-bromophenyl)-7-(3-bromo-4-(dimethylamino)phenyl)pyrido[2,3-d]pyrimidine; 4-amino-5-(3-bromophenyl)-7-(3-methyl-4-(dimethylamino)phenyl)pyrido[2,3-d]pyrimidine; 4-amino-5-(3-bromophenyl)-7-(4-(N-methyl-N-trifluoroacetylamino)phenyl)pyrido[2,3-d]pyrimidine; 4-amino-5-(3-bromophenyl)-7-(4-(dimethylamino)-3-fluorophenyl)pyrido[2,3-d]pyrimidine; 4-amino-5-(3-bromophenyl)-7-(4-(N-ethyl-N-formylamino)phenyl)pyrido[2,3-d]pyrimidine; 4,4-bis(acetylamino)-5-(3-bromophenyl)-7-(4-(N-methyl-N-acetylamino)phenyl)pyrido[2,3-d]pyrimidine; 4-amino-5-(3-bromophenyl)-7-(4-(N-acetyl-N-methylamino)phenyl)pyrido[2,3-d]pyrimidine; 4-amino-5-(3-bromophenyl)-7-(4-(N-ethylamino)phenyl)pyrido[2,3-d]pyrimidine; 4-amino-5-(3-bromophenyl)-7-(4-(N-methyl-N-(2-methoxyethyl)amino)phenyl)pyrido[2,3-d]pyrimidine; 4-amino-5-(3-bromophenyl)-7 (4-(N-isopropylamino)phenyl)pyrido[2,3-d]pyrimidine; 4-amino-5-(3-bromophenyl)-7-(4-N-ethyl-N-(2-methoxyethyl)amino)phenyl)pyrido[2,3-d]pyrimidine; 4-amino-5-(3-bromophenyl)-7-(4-N-(3-methoxypropionyl)-N-isopropyl-amino)phenyl)pyrido[2,3-d]pyrimidine; 4-amino-5-(3-bromophenyl)-7-(4-N-(2-(dimethylamino)ethyl)-N-formylamino)phenyl)pyrido[2,3-d]pyrimidine; 4-amino-5-(3-bromophenyl)-7-(4-(N-(2-(dimethylamino)ethyl)amino)phenyl)pyrido[2,3-d]pyrimidine; 4-amino-5-(3-bromophenyl)-7-(4-(N-methyl-N-(2-cyano)ethylamino)phenyl)pyrido[2,3-d]pyrimidine; 4-amino-5-(3-bromophenyl)-7-(4-(N-methyl-N-(3-methoxy)propionylamino)phenyl)pyrido[2,3-d]pyrimidine; 4-amino-5-(3-bromophenyl)-7-(3-methyl-4-(N-formyl-N-methylamino)phenyl)pyrido[2,3-d]pyrimidine; 4-amino-5-(3-bromophenyl)-7-(3-methyl-4-(N-methylamino)phenyl)pyrido[2,3-d]pyrimidine; 4-amino-5-(3-bromophenyl)-7-(4-methoxy-2-butyl)phenyl)pyrido[2,3-d]pyrimidine; 4-amino-5-(3-bromophenyl)-7-(4-(N-methyl-N-(2-(N-phthalimidyl)acetyl)amino)phenyl)pyrido[2,3-d]pyrimidine; 4-amino-S-(3-bromophenyl)-7-(3-methyl-4-(N-methyl-N-(trifluoroacetyl)amino)phenyl)pyrido[2,3-d]pyrimidine; 4-amino-5-(3-bromophenyl)-7-(3-methyl-4-(N-acetyl-N-methylamino)phenyl)pyrido[2,3-d]pyrimidine; 4-amino-5-(3-bromophenyl)-7-(6-dimethylamino-3-pyridinyl)pyrido[2,3-d]pyrimidine; 4-amino-5-(3-cyanophenyl)-7-(4-methylsulfonylphenyl)pyrido[2,3-d]pyrimidine; 4-amino-5-(3-cyanophenyl)-7-(4-(N-methyl-N-formylamino)-phenyl)pyrido[2,3-d]pyrimidine; 4-amino-5-(3-bromophenyl)-7-(6-(N-methyl-N-formylamino)-3-pyridinyl)pyrido[2,3-d]pyrimidine; 4-amino-5-(3-bromophenyl)-7-(6-morpholinyl-3-pyridinyl)-pyrido[2,3-d]pyrimidine; 4-amino-5-(3-bromophenyl)-7-(6-(N-methyl-N-methoxyethylamino)-3-pyridinyl)pyrido[2,3-d]pyrimidine; 4-amino-5-(3-bromophenyl)-7-(6-pyrrolidinyl-3-pyridinyl)pyrido[2,3-d]pyrimidine; 4-amino-5-(3-bromophenyl)-7-(2-(dimethylamino)-5-pyrimidinyl)pyrido[2,3-d]pyrimidine; 4-amino-5-(3-bromophenyl)-7-(2-(N-methoxyethyl-N-methylamino)-5-pyrimidinyl)pyrido[2,3-d]pyrimidine; 4-amino-S-(3-bromophenyl)-7-(2-(N-formyl-N-methylamino)-5-pyrimidinyl)pyrido[2,3-d]pyrimidine; 4-amino-5-(3-bromophenyl)-7-(2-(N-methylamino)-5-pyrimidinyl)pyrido[2,3-d]pyrimidine; 4-amino-5-(3-bromophenyl)-7-(2-(1-pyrrolidinyl)-5-pyrimidinyl)pyrido[2,3-d]pyrimidine; 4-amino-5-(3-bromophenyl)-7-(2-(1-morpholinyl)-5-pyrimidinyl)pyrido[2,3-d]pyrimidine; 4-amino-5-(3-bromophenyl)-7-(6-(2-oxo-3-oxazolidinyl)-3-pyridinyl)pyrido[2,3-d]pyrimidine; 4-amino-5-(3-bromophenyl)-7-(2-pyridyl)pyrido[2,3-d]pyrimidine; 4-amino-5-(3-bromophenyl)-7-(3-pyridyl)pyrido[2,3-d]pyrimidine; 4-amino-5-(3-(thiophen-2-yl)phenyl)-7 (4-dimethylaminophenyl)pyrido[2,3-d]pyrimidine; 4-amino-5-(3-(furan-2-yl)phenyl)-7-(4-dimethylaminophenyl)pyrido[2,3-d]pyrimidine; 4-amino-5-(3-(3-methoxyphenyl)phenyl)-7-(4-dimethylaminophenyl)pyrido[2,3-d]pyrimidine; 4-amino-S-phenyl-(4-dimethylaminophenyl)pyrido[2,3-d]pyrimidine; 4-amino-5-(3-chlorophenyl)-7-(4-(morpholinyl)phenyl)pyrido[2,3-d]pyrimidine; 4-amino-5-(3-bromo-4-fluorophenyl)-7-(4-(morpholinyl)phenyl)pyrido[2,3-d]pyrimidine; 4-amino-S-(3-chlorophenyl)-7-(4-iodophenyl)pyrido[2,3-d]pyrimidine; 4-amino-5-(3-chlorophenyl)-7-(4-(thiophen-2-yl)phenyl)pyrido[2,3-d]pyrimidine; 4-amino-5-(3-chlorophenyl)-7-(4-(5-pyrimidinyl)phenyl)pyrido[2,3-d]pyrimidine; 4-amino-5-(3-bromo-4-fluorophenyl)-7-(4-iodophenyl)pyrido[2,3-d]pyrimidine; 4-amino-5-(4-bromothiophene-2-yl)-7-(4-methoxyphenyl)pyrido[2,3-d]pyrimidine; 4-amino-5-(3-bromophenyl)methyl-7-(4-(dimethylamino)phenyl)pyrido[2,3-d]pyrimidine; 4-amino-5-(2-phenylethyl)-7-(4-diethylaminophenyl)pyrido[2,3-d]pyrimidine; 4-amino-5-(2-methylpropyl)-7-(4-diethylaminophenyl)pyrido[2,3-d]pyrimidine; 4-amino-5-(butyl)-7-(4-diethylaminophenyl)pyrido[2,3-d]pyrimidine; 4-amino-5-(2-(4-bromophenyl)ethyl)-7-(4-diethylaminophenyl)pyrido[2,3-d]pyrimidine; 4-amino-5-(butyl)-7-(4-dimethylaminophenyl)pyrido[2,3-d]pyrimidine; 4-amino-5-(2-(3-cyanophenyl)methyl-7-(4-dimethylaminophenyl)pyrido[2,3-d]pyrimidine; 4-amino-S-(2-(N-phenylmethoxycarbonyl)aminoethyl)-7-(4-dimethylaminophenyl)pyrido[2,3-d]pyrimidine; 4-amino-5-(cycloheptyl)-7-(4-dimethylaminophenyl)pyrido[2,3-d]pyrimidine; 4-amino-5-(2-(5-chloro-2-(thiophen-3-yl)phenylmethyl)-7-(4-dimethylaminophenyl)pyrido[2,3-d]pyrimidine; 4-amino-5-(pentyl)-7-(4-diethylaminophenyl)pyrido[2,3-d]pyrimidine; 4-amino-5-hexyl-7-(4-diethylaminophenyl)pyrido[2,3-d]pyrimidine; 4-amino-5-(2-(3-bromophenyl)ethyl)-7-(4-diethylaminophenyl)pyrido[2,3-d]pyrimidine; 4-amino-5-((2-bromophenyl)methyl)-7-(4-diethylaminophenyl)pyrido[2,3-d]pyrimidine; 4-amino-5-cyclopropyl-7-(4-dimethylaminophenyl)pyrido[2,3-d]pyrimidine; 4-amino-5-cyclohexyl-7-(4-dimethylaminophenyl)pyrido[2,3-d]pyrimidine; 4-amino-5-(2-bromo-5-chlorophenyl)methyl)-7-(4-diethylaminophenyl)pyrido[2,3-d]pyrimidine; 4-amino-5-methyl 7-(4-diethylaminophenyl)pyrido[2,3-d]pyrimidine; 4-amino-5-(2,3-methylenedioxyphenyl)-7-(4-dimethylaminophenyl)pyrido[2,3-d]pyrimidine; 4-amino-5-(3-fluoro-5-trifluoromethylphenyl)-7-(4-dimethylaminophenyl)pyrido[2,3-d]pyrimidine; 4-amino-5-(2-bromophenyl)-7-(4-dimethylaminophenyl)pyrido[2,3-d]pyrimidine; 4-amino-5-(3,5-dimethylphenyl)-7-(4-dimethylaminophenyl)pyrido[2,3-d]pyrimidine; 4-amino-5-(3,4-dichlorophenyl)-7-(4-dimethylaminophenyl)pyrido[2,3-d]pyrimidine; 4-amino-5-(4-fluoro-3-trifluoromethylphenyl)-7-(4-dimethylaminophenyl)pyrido[2,3-d]pyrimidine; 4-amino-5-(3-bromo-5-methoxyphenyl)-7-(4-morpholinylphenyl)pyrido[2,3-d]pyrimidine; 4-amino-5-(3-bromo-5-methoxyphenyl)-7-(4-pyrrolidinylphenyl)pyrido[2,3-d]pyrimidine; 4-amino-5-(3-bromo-5-methoxyphenyl)-7-(4-piperidinylphenyl)pyrido[2,3-d]pyrimidine; 4-amino-5-(3-bromo-5-methoxyphenyl)-7-(4-dimethylaminophenyl)pyrido[2,3-d]pyrimidine; 4-amino-5-(3-methylthiophenyl)-7-(4-dimethylaminophenyl)pyrido[2,3-d]pyrimidine; 4-amino-5-(3-bromo-5-methoxyphenyl)-7-(thiophene-2-yl)pyrido[2,3-d]pyrimidine; 4-amino-5-(2,3-dimethOxyphenyl)-7-(4-dimethylaminophenyl)pyrido[2,3-d]pyrimidine; 4-amino-5-(3-methylsulfonylphenyl)-7-(4-dimethylaminophenyl)pyrido[2,3-d]pyrimidine; 4-acetylamino-5-(3-bromophenyl)-7-(4-dimethylaminophenyl)pyrido[2,3-d]pyrimidine; 4-formylamino-5-(3-bromophenyl)-7-(4-dimethylaminophenyl)pyrido[2,3-d]pyrimidine; 4-(methoxyacetyl)amino-5-(3-bromophenyl)-7-(4-diethylaminophenyl)pyrido[2,3-d]pyrimidine; 4-trifluoroacetylamino-5-(3-bromophenyl)-7-(4-dimethylaminophenyl)pyrido[2,3-d]pyrimidine; 4-pentanoylamino-5-(3-bromophenyl)-7-(4-dimethylaminophenyl)pyrido[2,3-d]pyrimidine; 4-benzoylamino-5-(3-bromophenyl)-7-(4-dimethylaminophenyl)pyrido[2,3-d]pyrimidine; 4-(N—BOC-glycyl)amino-5-(3-bromophenyl)-7-(4-dimethylaminophenyl)pyrido[2,3-d]pyrimidine; 4-(N-phthalimidylglycyl)amino-5-(3-bromophenyl)-7-(4-dimethylaminophenyl)pyrido[2,3-d]pyrimidine; 4-(ethoxycarbonyl)amino-5-(3-bromophenyl)-7-(4-dimethylaminophenyl)pyrido[2,3-d]pyrimidine; 4-(ethylaminocarbonyl)amino-S-(3-bromophenyl)-7-(4-dimethylaminophenyl)pyrido[2,3-d]pyrimidine; 4-allylamino-5-(3-bromophenyl)-7-(4-dimethylaminophenyl)pyrido[2,3-d]pyrimidine; 4-(2-(N,N-dimethylamino)ethylamino)-5-(4-bromophenyl)-7-(4-dimethylaminophenyl)pyrido[2,3-d]pyrimidine; 4-(4-(N,N-dimethylamino)butylamino)-5-(3-bromophenyl)-7-(4-dimethylaminophenyl)pyrido[2,3-d]pyrimidine; 4-(N-allyl-N-formylamino)-5-(4-dimethylaminophenyl)-7-(4-bromophenyl)pyrido[2,3-d]pyrimidine; 4-diacetylamino-5-(p-dimethylaminophenyl)-7-(4-bromophenyl)pyrido[2,3-d]pyrimidine; 4-amino-5-(3-bromophenyl)-7-(5-amino-2-pyridyl)pyrido[2,3-d]pyrimidine; 4-amino-5-(3-bromophenyl)-7-(5-dimethylamino-2-pyridyl)pyrido[2,3-d]pyrimidine; 4-amino-5-(3-bromophenyl)-7-(5-dimethylamino-2-pyrazinyl)pyrido[2,3-d]pyrimidine; 4-amino-5-(3-bromophenyl)-7-(2-oxobenzoxazolin-6-yl)pyrido[2,3-d]pyrimidine; 4-amino-5-(3-bromophenyl)-7-(1-methyl-2-oxobenzoxazolin-6-yl)pyrido[2,3-d]pyrimidine; 4-amino-5-((5-chloro-2-(3-methoxyphenyl)phenyl)methyl-7-(4-dimethylaminophenyl)pyrido[2,3-d]pyrimidine; 4-amino-5-((thiophene-2-yl)methyl)-7-(4-diethylaminophenyl)pyrido[2,3-d]pyrimidine; 4-amino-5-((thiophene-3-yl)methyl)-7-(4-diethylaminophenyl)pyrido[2,3-d]pyrimidine; 4-amino-5-((2-bromophenyl)methyl)-7-(4-dimethylaminophenyl)pyrido[2,3-d]pyrimidine; 4-amino-5-(3-bromophenyl)-7-(4-(N-formyl)-N-(2-methoxyethyl)amino)phenyl)pyrido[2,3-d]pyrimidine; 4-amino-5-(3-bromophenyl)-7-(4-(N-(2-methoxyethyl)amino)phenyl)pyrido[2,3-d]pyrimidine; 4-amino-5-(3-bromophenyl)-7-(4-(N-methyl-N-((2-dimethylamino)ethyl)amino)phenyl)pyrido[2,3-d]pyrimidine; 4-amino-5-(3-bromophenyl)-7-(4-(2-methoxy)acetylamino)ethyl)amino)phenyl)pyrido[2,3-d]pyrimidine; 4-amino-5-(3-bromophenyl)-7-((4-formylamino)phenyl)pyrido[2,3-d]pyrimidine; 4-amino-5-(3-bromophenyl)-7-(4-(2-dimethylamino)acetylamino)phenyl)pyrido[2,3-d]pyrimidine; 4-amino-5-(3-bromophenyl)-7-(4-(2-oxo-3-oxazolidinyl)phenyl)pyrido[2,3-d]pyrimidine; 4-amino-5-(3-bromophenyl)-7-(6-(2-propyl)-3-pyridinyl)pyrido[2,3-d]pyrimidine; 4-amino-5-(3-bromophenyl)-7-(3-methyl 4-pyrrolidinyl)phenyl)pyrido[2,3-d]pyrimidine; 4-amino-5-(3-bromophenyl)-7-(6-imidazolyl-3-pyridinyl)pyrido[2,3-d]pyrimidine; 4-amino-5-phenylmethyl-7-(4-diethylaminophenyl)pyrido[2,3-d]pyrimidine; 4-amino-5-(2-(3-aminopropynyl)phenylmethyl)-7-(4-diethylaminophenyl)pyrido[2,3-d]pyrimidine; 4-amino-5-(1-(3-bromophenyl)ethyl)-7-(4-diethylaminophenyl)pyrido[2,3-d]pyrimidine; 4-amino-5-(4-dimethylaminophenyl)-7-(4-bromophenyl)pyrido[2,3-d]pyrimidine; 4-amino-5-(2-furanyl)-7-(4-(N-morpholinyl)phenyl)pyrido[2,3-d]pyrimidine; 4-amino-5-(3-bromophenyl)-7-(2-dimethylamino-5-pyrimidinyl)pyrido[2,3-d]pyrimidine; 4-amino-5-(3-bromophenyl)-7-(4-(ureido)phenyl)pyrido[2,3-d]pyrimidine; 4-amino-5-(1-phenylmethyl-3-piperidinyl)-7-(4-diethylaminophenyl)pyrido[2,3-d]pyrimidine; 4-amino-5-(3-bromophenyl)-7-(6-(3-methyl-5-isoxazolyl))-3-pyridinyl)pyrido[2,3-d]pyrimidine; 4-amino-5-(3-bromophenyl)-7-(6-chloro-3-pyridinyl)pyrido[2,3-d]pyrimidine; 4-amino-5-(3-bromophenyl)-7-(6-methoxy-3-pyridinyl)pyrido[2,3-d]pyrimidine; 4-amino-5-(3-bromophenyl)-7-(6-(1,2,4-triazol-4-yl)-3-pyridinyl)pyrido[2,3-d]pyrimidine; amino-5-(3-bromophenyl)-7-(2-morpholinyl-5-pyrimidinyl)pyrido[2,3-d]pyrimidine; 4-amino-5-(2-thiazolyl)-7-(4-pyrrolidinylphenyl)-pyrido[2,3-d]pyrimidine; 4-amino-5-(3-bromophenyl)-7-(6-pyrazolyl-3-pyridinyl))-pyrido[2,3-d]pyrimidine; 4-amino-5-(3-bromophenyl)-7-(4-(1-methyl-ureido)phenyl)-pyrido[2,3-d]pyrimidine; 4-amino-5-(3-bromophenyl)-7-(4-(N-methyl-N-(2-pyrimidinyl)amino)phenyl)-pyrido[2,3-d]pyrimidine; 4-amino-5-(3-bromophenyl)-7-(3-fluoro-4-(N-formyl-N-methylamino)phenyl)-pyrido[2,3-d]pyrimidine; 4-formylamino-5-(3-bromophenyl)-7-(3-fluoro-4-(N-formyl-N-methylamino)phenyl)-pyrido[2,3-d]pyrimidine; 4-amino-5-(3-bromophenyl)-7-(4-(N-methyl-N-methylsulfonylamino)-phenyl)pyrido[2,3-d]pyrimidine; 4-amino-5-(3-bromophenyl)-7-(6-(N-methyl-N-methylsulfonylamino)-3-pyridinyl)pyrido[2,3-d]pyrimidine; 4-amino-5-(3-bromophenyl)-7-(1-methyl-5-indolinyl)pyrido[2,3-d]pyrimidine; 4-amino-5-(3-bromophenyl)-7-(1-methyl-5-benzimidazolyl)pyrido[2,3-d]pyrimidine; 4-amino-5-(3-bromophenyl)-7-(6-dimethylamino-3-pyridazinyl)pyrido[2,3-d]pyrimidine; 4-amino-5-(3bromophenyl)-7-(6-morpholinyl-3-pyridazinyl)pyrido[2,3-d]pyrimidine; 4-amino-5-(3-bromophenyl)-7-(6-pyrrolidinyl-3-pyridazinyl)pyrido[2,3-d]pyrimidine; 4-amino-5-(3-bromophenyl)-7-(5-morpholinyl-2-pyrazinyl)pyrido[2,3-d]pyrimidine; 4-amino-5-(3-bromophenyl)-7-(5-(N-(2-methoxyethyl)-N-methylamino) 2 pyrazinyl)pyrido[2,3-d]pyrimidine; 4-amino-5-(3-bromophenyl)-7-(4-(morpholinylmethyl)-phenyl)pyrido[2,3-d]pyrimidine; 4-amino-5-(3-bromophenyl)-7-(5-(N,N-bis(2-methoxyethyl)amino)-2-pyridinyl)pyrido[2,3-d]pyrimidine; 4-amino-5-(3-bromophenyl)-7-(4-(imidazolylmethyl)-phenyl)pyrido[2,3-d]pyrimidine; 4-amino-5-(3-bromophenyl)-7-(5-(4-morpholinyl)-2-pyridinyl)pyrido[2,3-d]pyrimidine; 4-amino-5-(3-bromophenyl)-7-(4-((dimethylamino)methyl)-phenyl)pyrido[2,3-d]pyrimidine; 4-amino-5-(3-bromophenyl)-7-(5-(4-hydroxy-1-piperidinyl)-2-pyridinyl)pyrido[2,3-d]pyrimidine; A-amino-5-(3-bromophenyl)-7-(5-N-formyl-N-methylamino)-2-pyridinyl)pyrido[2,3-d]pyrimidine; 4-amino-5-(3-bromophenyl)-7-(5-(2-propenyl)-2-pyridinyl)pyrido[2,3-d]pyrimidine; 4-amino-5-(3-bromophenyl)-7-(3-(2-methoxyethyl)-2-oxo-6-benzoxazolyl)pyrido[2,3-d]pyrimidine; 4-amino-5-(3-bromophenyl)-7-(4-(1-(N-formylamino)-ethyl)phenyl)pyrido[2,3-d]pyrimidine; 4-(methylamino)-5-(3-bromophenyl)-7-(4-dimethylaminophenyl)pyrido[2,3-d]pyrimidine hydrochloride; 4-(2-methoxyethylamino)-5-(3-bromophenyl)-7-(4-dimethylaminophenyl)pyrido[2,3-d]pyrimidine hydrochloride; 4-amino-5-(3-bromophenyl)-7-(4-(1-methyl-2-imidazolyl)phenyl)pyrido[2,3-d]pyrimidine trihydrochloride; 4-amino-5-(3-bromophenyl)-7-(4-(aminomethyl)phenyl)pyrido[2,3-d]pyrimidine; 4-amino-5-(3-bromophenyl)-7-(2-bromo-4-(dimethylamino)phenyl)pyrido[2,3-d]pyrimidine; 4-amino-5-(3-bromophenyl)-7-(4-(dimethylaminoethyl)phenyl)pyrido[2,3-d]pyrimidine; 4-amino-5-(3-bromophenyl)-7-(4-(3-(dimethylamino)propynyl)phenyl)pyrido[2,3-d]pyrimidine; 4-amino-5-(3-bromophenyl)-7-(4-(3-amino-3-methylbutynyl)phenyl)pyrido[2,3-d]pyrimidine; 4-amino-5-(3-bromophenyl)-7-(4-dimethylphosphonatophenyl)pyrido[2,3-d]pyrimidine; 4-amino-5-(3-bromophenyl)-7-(4-(3-(methoxypropynyl)pyrido[2,3-d]pyrimidine; 4-amino-5-(3-bromophenyl)-7-(4-(carboxyphenyl)pyrido[2,3-d]pyrimidine; 4-amino-5-(3-bromophenyl)-7-(4-methyl-3-oxo-2H-4H-pyrido[3,2-b]-1,4-oxazin-7-yl)pyrido[2,3-d]pyrimidine; 4-amino-5-(3-bromophenyl)-7-(4-(2-(dimethylamino)ethyl)-3-oxo-2H-4H-pyrido[3,2-b]-1,4-oxazin-7-yl)pyrido[2,3-d]pyrimidine; 4-amino-5-(3-bromophenyl)-7-(2,3-dihydro-3-(dimethylaminoethyl)-2-oxobenzoxazol-6-yl)pyrido[2,3-d]pyrimidine; 4-amino-5-(3-bromophenyl)-7-(4-methyl-3-oxo-2H-4H-benzo-1,4-oxazin-7-yl)pyrido[2,3-d]pyrimidine; 4-amino-5-(3-bromophenyl)-7-(2,2,4-trimethyl-3-oxo-2H-4H-benzo-1,4-oxazin-7-yl)pyrido[2,3-d]pyrimidine; 4-amino-5-cyclohexyl-7-(4-(2-dimethylamino)ethyl)-2H-4H-benzo-3-oxo-1,4-oxazin-7-yl)pyrido[2,3-d]pyrimidine; 4-amino-5-(3-bromophenyl)-7-(5-(1-methylethyl)-2-pyridyl)pyrido[2,3-d]pyrimidine; 4-amino-5-(3-bromophenyl)-7-(5-piperidin-1-ylpyrid-2-yl)pyrido[2,3-d]pyrimidine; 4-amino-5-(1-(4-bromophenyl)ethyl)-7-(6-morpholinylpyrid-3-yl)pyrido[2,3-d]pyrimidine; 4-amino-5-(3-bromophenyl)-7-(4-((N-formylamino)methyl)phenyl)pyrido[2,3-d]pyrimidine; 4-amino-5-(3-bromophenyl)-7-(4-(1-methyl-1-(N-methylamino)ethyl)phenyl)-pyrido[2,3-d]pyrimidine; 4-amino-5-(3-bromophenyl)-7-(4-(1-(dimethylamino)-1-methylethyl)phenyl)pyrido[2,3-d]pyrimidine; 4-amino-5-(3-bromophenyl)-7-(N-acetyl-5-indolinyl)pyrido[2,3-d]pyrimidine; 4-amino-5-cyclohexyl-7-(6-chloro-3-pyridyl)pyrido[2,3-d]pyrimidine; 4-amino-5-(1-(2-bromophenyl)ethyl)-7-(6-diethylamino-3-pyridyl)pyrido[2,3-d]pyrimidine; 4-amino-5-(1-(2-bromophenyl)ethyl)-7-(6-morpholinyl-3-pyridyl)pyrido[2,3-d]pyrimidine; 4-amino-5-(1-(2-bromophenyl)ethyl)-7-(4-(N-methyl-N-formyl)amino)-phenyl)pyrido[2,3-d]pyrimidine; 4-amino-5-cyclohexyl-7-(6-morpholinyl-3-pyridyl)pyrido[2,3-d]pyrimidine; 4-amino-5-((2-bromophenyl)methyl)-7-(6-morpholinyl-3-pyridyl)pyrido[2,3-d]pyrimidine; 4-amino-5-(4-tetrahydropyranyl)-7-(6-morpholinyl-3-pyridyl)pyrido[2,3-d]pyrimidine; 4-amino-5-cyclohexyl-7-(6-dimethylamino-3-pyridyl)pyrido[2,3-d]pyrimidine; 4-amino-5-(1-ethylpropyl)-7-(6-dimethylamino-3-pyridyl)pyrido[2,3-d]pyrimidine; 4-amino-5-cyclopentyl-7-(6-morpholinyl-3-pyridyl)pyrido[2,3-d]pyrimidine; 4-amino-5-cyclohexyl-7-(2-chloro-3-pyridyl)pyrido[2,3-d]pyrimidine; 4-amino-5-(3,5-dimethylcyclohexyl)-7-(6-dimethylamino-3-pyridyl)pyrido[2,3-d]pyrimidine; 4-amino-5-((N-(benzyloxycarbonyl)-4-piperidinyl)methyl)-7-(6-morpholinyl-3-pyridyl)pyrido[2,3-d]pyrimidine; 4-amino-5-cyclohexyl-7-(6-bromo-3-pyridyl)pyrido[2,3-d]pyrimidine; 4-amino-5-cyclohexyl-7-(3-cyanophenyl)pyrido[2,3-d]pyrimidine; 4-amino-5-(1-(2-bromophenyl)ethyl)-7-(6-dimethylamino-3-pyridazinyl)pyrido[2,3-d]pyrimidine; 4-amino-5-(3-bromophenyl)-7-(6-imidazolyl-3-pyridazinyl)pyrido[2,3-d]pyrimidine; 4-amino-5-(3-bromophenyl)-7-(6-(azacycloheptanyl)-3-pyridazinyl)pyrido[2,3-d]pyrimidine; 4-amino-5-(3-bromophenyl)-7-(6-(N-methyl-N-(1-methylethyl))amino)-3-pyridazinyl)pyrido[2,3-d]pyrimidine; 4-amino-5-(1-(2-bromophenyl)ethyl)-7-(6-morpholinyl-3-pyridazinyl)pyrido[2,3-d]pyrimidine; 4-amino-5-cyclohexyl-7-(6-(4-acetylpiperazinyl)-3-pyridyl)pyrido[2,3-d]pyrimidine; 4-amino-5-cyclohexyl-7-(6-(4-acetyl-1,4-diazacycloheptanyl)-3-pyridyl)pyrido[2,3-d]pyrimidine; 4-amino-5-cyclohexyl-7-(6-(4-methyl-1,4-diazacycloheptanyl)-3-pyridyl)pyrido[2,3-d]pyrimidine; 4-amino-5-cyclohexyl-7-(6-(N-methyl-N-(2-(2-pyridyl)ethyl)amino)-3-pyridyl)pyrido[2,3-d]pyrimidine; 4-amino-5-cyclohexyl-7-(6-2-(N—(N′,N-dimethylaminoethyl)-N-methylamino)-3-pyridyl)pyrido[2,3-d]pyrimidine; 4-amino-5-cyclohexyl-7-(6-azetidinyl-3-pyridyl)pyrido[2,3-d]pyrimidine; 4-amino-5-cyclohexyl-7-(6-(3-(N-methylacetamido)pyrrolidinyl)pyridyl)pyrido[2,3-d]pyrimidine; 4-amino-5-cyclohexyl-7-(6-(3-(formamido)pyrrolidinyl)pyridyl)pyrido[2,3-d]pyrimidine; 4-amino-5-cyclohexyl-7-(4-oxo-1-phenyl-1,3,8-triazaspiro[4,5[decan-8-yl)pyrido[2,3-d]pyrimidine; 4-amino-5-cyclohexyl-7-(6-(2-methoxymethyl)pyrrolidin-1-yl)pyridyl)pyrido[2,3-d]pyrimidine; 4-amino-5-cyclohexyl-7-(6-(N-methoxyethyl-N-propylamino)pyridyl)pyrido[2,3-d]pyrimidine; 4-amino-5-cyclohexyl-7-(N-methyl-N-(2,2-dimethoxyethyl)amino)pyrido[2,3-d]pyrimidine; 4-amino-5-cyclohexyl-7-(6-(4-(dimethylamino)piperidinyl)pyridyl)pyrido[2,3-d]pyrimidine; 4-amino-5-cyclohexyl-7-(6-(4-(aminocarbonyl))piperidinyl)pyridyl)pyrido[2,3-d]pyrimidine; 4-amino-5-cyclohexyl-7-(N-methyl-N-(3-(diethylamino)propyl)aminopyrid-3-yl)pyrido[2,3-d]pyrimidine; 4-amino-5-cyclohexyl-7-(6-(N-methyl-N-(4-pyridyl)ethylamino)pyrid-3-yl)pyrido[2,3-d]pyrimidine; 4-amino-5-cyclohexyl-7-(6-(N-methyl-N-(3-pyridylmethylamino)pyrid-3-yl)pyrido[2,3-d]pyrimidine; 4-amino-5-(1-(2-bromophenyl)ethyl)-7-(1-methyl-S-indolyl)pyrido[2,3-d]pyrimidine; 4-amino-5-(1-(2-bromophenyl)ethyl)-7-(1-methyl-2,3-dioxo-S-indolyl)pyrido[2,3-d]pyrimidine, 4-amino-5-(3-bromophenyl)-7-(3-fluoro-4-(1-morpholinyl)phenyl)pyrido[2,3-d]pyrimidine; 4-amino-5-(3-bromophenyl)-7-(4-hydroxy-3-nitrophenyl)pyrido[2,3-d]pyrimidine; 4-amino-5-(3-bromophenyl)-7-(6-(4,4-ethylenedioxypiperidinyl)-3-pyridyl)pyrido[2,3-d]pyrimidine; 4-amino-5-(3-bromophenyl)-7-(6-(4-oxopiperidinyl)-3-pyridyl)pyrido[2,3-d]pyrimidine; 4-amino-5-(3-bromophenyl)-7-(6-(4-formylpiperazinyl)-3-pyridyl)pyrido[2,3-d]pyrimidine; 4-amino-5-(3-bromophenyl)-7-(6-(4-methylpiperazinyl)-3-pyridyl)pyrido[2,3-d]pyrimidine; 4-amino-5-(3-bromophenyl)-7-(6-thiomorpholinyl-3-pyridyl)pyrido[2,3-d]pyrimidin; 4-amino-5-(3-bromophenyl)-7-(6-(4,4-dioxothiomorpholinyl)-3-pyridyl)pyrido[2,3-d]pyrimidine; 4-amino-5-(2-bromophenyl)-7-(6-morpholinyl-3-pyridyl)pyrido[2,3-d]pyrimidine; 4-amino-5-(3-bromo-4-methoxyphenyl)-7-(6-morpholinyl-3-pyridyl)pyrido[2,3-d]pyrimidine; 4-amino-5-(4-bromophenyl)-7-(6-morpholinyl-3-pyridyl)pyrido[2,3-d]pyrimidine; 4-amino-5-(3-chlorophenyl)-7-(6-morpholinyl-3-pyridyl)pyrido[2,3-d]pyrimidine; 4-amino-5-(3-bromophenyl)-7-(5-chloro-6-morpholinyl-3-pyridyl)pyridyl)pyrido[2,3-d]pyrimidine; 4-amino-5-(3-bromophenyl)-7-(6-(N-oxidomorpholinyl)-3-pyridyl)pyrido[2,3-d]pyrimidine; 4-amino-5-(3-bromophenyl)-7-(6-(N-(2-hydroxyethoxyethyl)amino)-3-pyridyl)pyrido[2,3-d]pyrimidine; 4-amino-5-(3-bromophenyl)-7-(6-(N-(2-hydroxyethoxyethyl)-N-formylamino)-3-pyridyl)pyrido[2,3-d]pyrimidine; 4-amino-5-(3-bromophenyl)-7-(6-(N-(2-hydroxyethoxyethyl)-3-pyridyl-N-oxide)pyrido[2,3-d]pyrimidine; 4-amino-5-(3-bromophenyl)-7-(6-(3-hydroxy)morpholinyl)-3-pyridyl)pyrido[2,3-d]pyrimidine; 1-(5-(4-amino-5-(3-bromophenyl)pyrido[2,3-d]pyrimidin-7-yl)-2-pyridyl)-piperidine-4-phosphate, disodium salt; 4-amino-5-(3-bromophenyl)-7-(4-methylenylpiperidinyl)-3-pyridyl)pyrido[2,3-d]pyrimidine; 4-amino-5-(3-bromophenyl)-7-(4-hydroxy-4-(hydroxymethyl)piperidinyl)-3-pyridyl)pyrido[2,3-d]pyrimidine; 4-amino-5-(3-bromophenyl)-7-(6-(4,4-ethylenedioxypiperidinyl)-3-pyridyl)pyrido[2,3-d]pyrimidine; 4-amino-5-cyclohexyl-7-(6-(4-oxo-piperidinyl)-3-pyridyl)pyrido[2,3-d]pyrimidine; 4-amino-S-cyclohexyl-7-(6-(4-methylenylpiperidinyl)-3-pyridyl)pyrido[2,3-d]pyrimidine; 4-N-(iminomethyl)amino 5-cyclohexyl-7-(6-dimethylamino-3-pyridyl)pyrido[2,3-d]pyrimidine; and pharmaceutically acceptable salts thereof.

Compounds of formula (II) can be synthesized using methods known in the art and easily available to the skilled artisan. For example, preparation of 5,7-disubstituted and 5,6,7-trisubstituted 4-aminopyrido[2,3-d]pyrimidine compounds is described in Int. Pat. App. Pub. No. WO98/46605 and U.S. Pat. No. 6,0303,969, contents of which are herein incorporated by reference in their entirety. Victory, P. et al., Tetrahedron (1995), 51, 10253-10258, contents of which are herein incorporated by reference, describes the synthesis of 4-amino-5,7-diphenylpyrido[2,3-d]pyrimidine compounds.

Other adenosine kinase inhibitors amenable to the invention are described in U.S. Pat. Nos. 5,506,347; 5,763,696; 5,763,597; 5,674,998; 5,721,356; 5,726,302; 5,795,977; and 5,864,033, contents of which are herein incorporated by reference in their entirety.

S-Adenosylhomocysteine hydrolase (SAHH) is a ubiquitous cellular enzyme that controls intracellular levels of S-adenosylhomocysteine (SAH). SAHH is also known as AA987153, Adenosylhomocysteinase, AdoHcyase, AL024110, CuBP, CUBP, Liver copper-binding protein, MGC102079, MGC118063, MGC19228, S-adenosyl-L-homocysteine hydrolase, and SAH hydrolase in the art. SAHH catalyzes the hydrolysis of S-adenosylhomocysteine to adenosine and homocysteine. SAH is a potent product inhibitor of some S-adenosylmethionine-dependent methyltransferases and inhibition of SAHH results in inhibition of S-adenosyl-L-methionine (SAM)-dependent methylation reactions.

S-Adenosylhomocysteine Hydrolase inhibitors include, but are not limited to adenosine and analogues and derivatives thereof. Exemplary adenosine analogues and derivatives include, but are not limited to, 9(S)-(2,3-dihydroxypropyl)adenine [(S)-DHPA]; D-eritadine; (R,S)-3-adenine-9-yl-2-hydroxypropanoic acid [(R,S)-AHPA]; adenosine (Ado) dialdehyde; 3-deazaadenosine (c3-Ado); aristeromycin (Ari) and analogs; neplanocin A (NPA or NpcA); dihydroxycyclopentenyladenine (DHCeA); dihydroxycyclopentenyl-3-deazaadenine (c3-DHCeA); dihydroxycyclopentenyladenine (DHCaA); dihydroxycyclopentanyl-3-deazaadenine (c3-DHCaA); 3-deazaneplanocin A (c3-NpcA); 3-deazaaristeromycin (c3Ari); carbocyclic-3-deazaadenosine (C-c3Ado); 6′-Cmethylneplanocin A; 2′-deoxyadenosine; tubercidin; ribavirin; pyraazofurin; 2′-deoxy-2′-chloroadenosine; isopentenyladenosine; methylthioadenosine (MTA); 9-β-arabinofuranosyladenine (Ara-A, vidarabine); 2′-Deoxyadenosine; N-methylaristeromycin, 8-azaaristeromycin and 3-deazaaristeromycin, and their dialdehyde and diol derivatives; (±)-5Noraristeromycin and its 2,6-diamino-analogue. Aristeromycin analogues and derivatives include, but are not limited to, 2′-deoxy-, 3′-deoxy-, 3′-amino-3′-deoxy-, 3′-amino-3′-deoxyarabinofuranosyl, 6′-hydroxy, 6′-mercapto, 8′-bromo, 8-hydroxyaristeromycin, aristeromycin-3′-cyclic phosphate and aristeromycin-6′-cyclic phosphate.

Certain structural analogues of S-Adenosylhomocysteine (SAH) with modification in the amino acid, base or sugar portion of the molecule can also used as SAH hydrolase inhibitors. Exemplary SAH analogues include, but are not limited to, 2-fluoro-S-adenosylhomocysteine (2-FSAH), S-Adenosyl-L-homocysteine sulfoxide, S-Adenosyl-Lhomocysteine sulfone, S-aristeromycinyl-L-homocysteine, 5′-S-(3-carboxyl-4-nitrophenyl)thioadenosine and 5′-S(methyl)-5′-S-(butyl)thioadenosine.

Other inhibitors of SAHH include those described, for example, in Yuan et al., Exp. Opin. Ther. Patents, 1999, 9: 1197-1206; Wolfe and Borchardt, Journal of Medicinal Chemistry, 1991, 34:1521-1530); Votruba and Holy, Coll Czech. Chem. Commun., 1980, 45:3039; Schanche et al., Molecular Plarmacology, 1984, 26:553-558; De Clercq E., Nucleosides Nucleotides, 1998, 17(1-3):625-34; and U.S. patent application Ser. No. 10/410,879 content of all of which is herein incorporated by reference in its entirety.

AMP-activated protein kinase can be activated allosterically by increases in the concentration of AMP or by a compound that is analogous to AMP. For example the AMP analog can be adenosine-5′-thiomonophosphate, adenosine 5′-phosphoramidate, formycin A 5′-monophosphate, or 5′-monophosphate-5-aminoimidazole-4-carboxamide ribonucleoside (ZMP).

In some embodiments of this and other aspects of the invention, the AMP activated protein kinase activator is of formula (III):

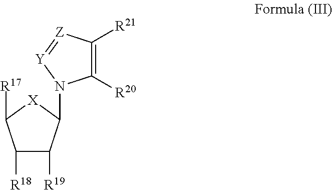

wherein:

R¹⁷ is H, optionally substituted C₁-C₆ alkyl, optionally substituted C₂-C₆ alkenyl, optionally substituted C₂-C₆ alkynyl, OR²², SR²², N(R²²)₂, (CH₂)ₘR²³, or R¹⁷ and R¹⁸ together with the atoms they are attached to form 5-8 membered heterocycle which can be optionally substituted;

R¹⁸ and R¹⁹ are each independently H, OR²², SR²², N(R²²)₂, O or R¹⁸ and R¹⁹ together with the atoms they are attached to form 5-8 membered heterocycle which can be optionally substituted;

R²⁰ and R²¹ are each independently halogen, CN, N₂, OR²², SR²², N(R²²)₂, C(O)R²⁴, C(O)OR²⁴, C(O)N(R²⁴)₂, optionally substituted C₁-C₆ alkyl, optionally substituted C₂-C₆ alkenyl, optionally substituted C₂-C₆ alkynyl, optionally substituted aryl, optionally substituted heteroaryl, optionally substituted cyclyl, optionally substituted heterocyclyl, optionally substituted arylalkyl, or optionally substituted heteroarylalkyl;

R²² is independently for each occurrence H, C(O)R²⁴, C(O)OR²⁴, C(O)N(R²⁴)₂, optionally substituted C₁-C₆ alkyl, optionally substituted C₂-C₆ alkenyl, optionally substituted C₂-C₆ alkynyl, optionally substituted aryl, optionally substituted heteroaryl, optionally substituted cyclyl, optionally substituted heterocyclyl, optionally substituted arylalkyl, or optionally substituted heteroarylalkyl;

R²³ is R²², OR²², SR²², N(R²²)₂, N₂, CN, halogen, or

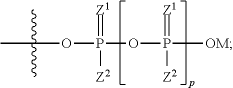

R²⁴ is independently for each occurrence H, optionally substituted C₁-C₆ alkyl, optionally substituted C₂-C₆ alkenyl, optionally substituted C₂-C₆ alkynyl, optionally substituted aryl, optionally substituted heteroaryl, optionally substituted cyclyl, optionally substituted heterocyclyl, optionally substituted arylalkyl, or optionally substituted heteroarylalkyl;

X is O, S, NH, or CH₂;

Y and Z are each independently N or CR²⁵;

R²⁵ is independently for each occurrence H, halogen, CN, C(O)R²⁴, C(O)OR²⁴C(O)N(R²⁴)₂, optionally substituted C₁-C₆ alkyl, optionally substituted C₂-C₆ alkenyl, optionally substituted C₂-C₆ alkynyl, optionally substituted aryl, optionally substituted heteroaryl, optionally substituted cyclyl, optionally substituted heterocyclyl, optionally substituted arylalkyl, or optionally substituted heteroarylalkyl;

Z¹ is independently for each occurrence O or S;

Z² is independently for each occurrence H, OM, SM, OR²², SR²², N(R²²)₂, optionally substituted C₁-C₆ alkyl, optionally substituted C₂-C₆ alkenyl, optionally substituted C₂-C₆ alkynyl, optionally substituted aryl, optionally substituted heteroaryl, optionally substituted cyclyl, optionally substituted heterocyclyl, optionally substituted aryl alkyl, or optionally substituted heteroarylalkyl;

M is an alkali metal cation;

m is 1, 2, 3, or 4;

n is 0, 1, or 2; and

pharmaceutically acceptable salts and amides thereof.

In some embodiments, M is Na⁺.

In some embodiments, R¹⁷ is optionally substituted C₁-C₆ alkyl, OR²², N(R²²)₂, or (CH₂)ₘR²³. Preferably C₁-C₆ alkyl is methyl. When R¹⁷ is N(R²²)², at least one of R²² is H, preferably both R²² are H. When R¹⁷ is OR²², R²² can be H or C₁-C₆ alkyl, preferably R²² is H.

When R¹⁷ is (CH₂)ₘR²³, m is 1 or 2, preferably m is 1. In some embodiments, R²³ is OR²² or N(R²²)₂. When R²³ is N(R²²)₂, at least one of R²² is H, preferably both R²² are H. When R²³ is OR³³, R³³ can be H or C₁-C₆ alkyl, preferably R⁵ is H. In some embodiments, R¹⁷ is CH₂OH.

In some embodiments, R¹⁷ and R¹⁸, in the compounds of formula (III), together with the atoms they are attached to form a 5-8 membered heterocycle, wherein the backbone of the heterocycle comprises

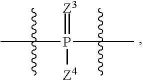

wherein Z³ is independently for each occurrence O or S and Z⁴ is H, OM, SM, OR²², SR²², N(R²²)₂, optionally substituted C₁-C₆ alkyl, optionally substituted C₂-C₆ alkenyl, optionally substituted C₂-C₆ alkynyl, optionally substituted aryl, optionally substituted heteroaryl, optionally substituted cyclyl, optionally substituted heterocyclyl, optionally substituted arylalkyl, or optionally substituted heteroarylalkyl. Preferably Z³ is O and Z⁴ is OM or SM.

In some other embodiments, X is selected from the group consisting of O, NH and CH₂. Preferably X is O.

In yet some other embodiments, R¹⁸ and R¹⁹ are both OR²². In some preferred embodiments, R¹⁸ and R¹⁹ are both OH.

In some embodiments, R²⁰ is selected from the group consisting of halogen, CN, OR²², optionally C₁-C₆ alkyl, N(R²²)₂, C(O)R²⁴, C(O)OR²⁴, and C(O)N(R²⁴)₂, wherein R²² and R²⁴ are as defined above. Preferably R²⁰ is NHR²², and more preferably R²⁰ is NH₂.

In some embodiments, R²¹ is selected from the group consisting of halogen, CN, OR²², C₁-C₆ alkyl, N(R²²)₂, C(O)R²⁴, C(O)OR²⁴, and C(O)N(R²⁴)₂, wherein R²² and R²⁴ are as defined above. Preferably R²¹ is C(O)NHR²⁴, and more preferably R²¹ is C(O)NH₂.

In some embodiments, R²⁰ is not NH₂ and R²¹ is not C(O)NH₂.

In some embodiments, at least one of Y or Z is CR²⁵, preferably Y is CR²⁵ and more preferably Y is CH₂. In some embodiments, Y is CR²⁵ and Z is N.

In some other embodiments, both of Y and Z are CR²⁵. Preferably Y is CH₂ and Z is CR²⁵, wherein R²⁵ is selected from the group consisting of CN, halogen, optionally substituted aryl, optionally substituted heteroaryl, optionally substituted arylalkyl, and optionally substituted heteroarylalkyl. Preferably the aryl group is a phenyl group, which is optionally substituted.

In some embodiments, the compound of formula (III) has the stereochemical configuration shown in formula (IIIa):

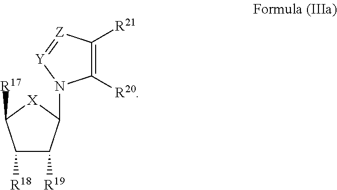

In some embodiments, the compound of formula (III) has the stereochemical configuration shown in formula (IIIb):

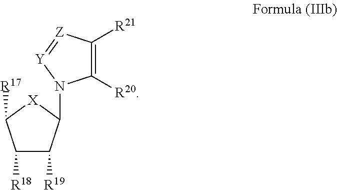

In some embodiments, the AMP-activated kinase activator is 5-aminoimidazole-4-carboxamide ribonucleoside (also referred to as AICAR herein).

Compounds of formula (III) can be prepared, for example by using methods described in U.S. Pat. Nos. 3,450,693 and 5,658,889, contents of which are herein incorporated by reference in their entirety. Mioyoshi et al., Chem. Pharm. Bull. (1976), 24:2089-2093; Chambers et. al., Nucleosides & Nucleotides (1988), 7: 339-346; and Srivastava et al. J. Org. Chem. (1975) 40:2920-2924, describe derivates and analogs of 5-aminoimidazole-4-carboxamide ribonucleoside.

Other AMPK activators amenable to the invention are described in U.S. Pat. No. 7,119,205 and U.S. Pat. Pub. Nos. 2006/0287356; 2007/0015665; and 2007/024420, contents of which are herein incorporated by reference in their entirety.

### Pancreatic Cells

As used herein, the term “pancreatic cells” refers to cells, or a population, or preparation of cells of pancreatic tissues, which can include both endocrine and exocrine tissues, as well as cell lines derived therefrom. The endocrine pancreas is composed of hormone producing cells arranged in clusters known as islets of Langerhans. Of the four main types of cells that form the islets (“islet cells”), the alpha cells produce glucagons, the beta cells produce insulin, the delta cells produce somatostatin, and the PP cells produce pancreatic polypeptide (PP). The exocrine pancreas includes the pancreatic acini and the pancreatic duct. Pancreatic acinar cells synthesize a range of digestive enzymes. Ductal cells secrete bicarbonate ions and water in response to the hormone secreted from the gastrointestinal tract. Therefore, the term “pancreatic cells” includes cells found in a pancreas, including alpha cells, beta cells, delta cells, PP cells, acinar cells, ductal cells, mesenchymal cells, fibroblasts and other cells present in the pancreatic connective tissue, or other cells (e.g., endothelial cells, neuronal cells, and progenitor cells that are not differentiated or not fully differentiated or yet to be differentiated), or a mixture or combination thereof.

Markers characteristic of pancreatic cells include the expression of cell surface proteins or the encoding genes, the expression of intracellular proteins or the encoding genes, cell morphological characteristics, and the production of secretory products such as glucagon, insulin and somatostatin. Those skilled in the art will recognize that known immunofluorescent, immunochemical, polymerase chain reaction, in situ hybridization, Northern blot analysis, chemical or radiochemical or biological methods can readily ascertain the presence or absence of islet cell specific characteristics.

If desired, the type(s) of cells in a population of pancreatic cells may be determined using techniques that are well known in the art. For example, the use of cell-type specific stains, such as, for example dithizone, that is specific for islet cells. Alternatively, one may perform immunofluorescence staining using antibodies directed to various pancreatic cell specific proteins, such as, for example, insulin, somatostatin, glucagon, pancreatic polypeptide cytokeratins, amylase, and lipase. In addition, a cell type may be determined by its morphology using techniques such as, for example, light microscopy, or electron microscopy.

In some embodiments, the pancreatic cells are from pancreatic endocrine tissues. In some embodiments, the pancreatic cells are within islet of Langerhans. The term “islet” or “islets” as used herein includes the constituent cell types within the islet of Langerhans, including alpha, beta, delta, and epsilon cells, intact islets, islet fragments or combinations thereof.

As used herein, the term “pancreatic cell” includes primary pancreatic cells, pancreatic cell like cells derived from dedifferentiated cells, e.g. from induced pluripotent stem cells (iPSCs), or pancreatic cell like cells that have been directly reprogrammed from a cell of endodermal origin (e.g. a liver cell or an exocrine pancreatic cell). In one embodiment, the pancreatic cell is not an immortalized cell line (e.g. proliferate indefinitely in culture). In one embodiment, the pancreatic cell is not a transformed cell, e.g, a cell that exhibits a transformation property, such as growth in soft agar, or absence of a contact inhibition.

The pancreatic cell population can be comprised of only one pancreatic cell type or a mixture of different pancreatic cell types. In some embodiments of this and other aspects of the invention described herein, the pancreatic cell population is comprised of a pancreatic cell type selected from the group consisting of alpha cells, beta cells, delta cells, epsilon cells, and combinations thereof. In some embodiments, pancreatic cell population is population of beta cells. In some embodiments, pancreatic cell populations also includes non-pancreatic cell types.

It is to be understood that when a pancreatic cell population comprises a mixture of different pancreatic cell types, the different cell types can be present in any ratio to each other. Without wishing to be bound by theory, each cell type in mixture can be present between 1-99% of the total cells. In some embodiments, pancreatic cell population comprises between 1-99% of beta cells to the total cells in the population. In some embodiments, pancreatic cell population comprises between 1-50% of beta cells to the total cells in the population.

In one embodiment, the pancreatic cells are primary pancreatic cells. In some embodiments, the pancreatic cells are primary pancreatic β-cells. In some embodiments, the pancreatic cells are not transformed pancreatic cells. In some embodiments, the pancreatic cells are not transformed pancreatic β-cells. In some embodiments, the pancreatic cells are not immortalized pancreatic cells. In some embodiments, the pancreatic cells are not immortalized pancreatic β-cells.

In some embodiments, the pancreatic cells are re-differentiated pancreatic cells. As used herein, the term “re-differentiated pancreatic cell” refers to a pancreatic cell that is differentiated from a de-differentiated pancreatic cell. In some embodiments, pancreatic cells are re-differentiated β-cells. As used herein, the term “re-differentiated β-cell” refers to a β-cell that is differentiated from a de-differentiated β-cell. A re-differentiated β-cell, can secret insulin in a glucose-regulated manner, has a β-cell type morphology, and is capable of forming adherens junctions. See e.g., Volk et al., Arch Pathol. 88(4): 413-22 (1969).

In some embodiments, the pancreatic cells are derived from de-differentiated somatic cells (e.g., reprogrammed cells). For example, a somatic cell de-differentiated to a pluripotent stem cell, or to a pancreatic cell (for example by direct reprogramming of a cell of endodermal origin). Without wishing to be bound by theory, a de-differentiated cell has a morphology that resembles a more primitive cell type from which it was derived, e.g., mesenchymal morphology.

Pancreatic cells can be also be derived (i.e. differentiated) from a subject's or donor's embryonic stem cells (ESCs). In some embodiments, induced pluripotent stem cells can be generated from a subject or a donor and then differentiated into pancreatic cells or pancreatic cell like cells. Induction of β-cell differentiation in human cells is described in U.S. Pat. Nos. 6,84,585; 6,911,324; and 7,276,352 and U.S. Pat. Pub. No. U.S. Pat. App. Pub. No. 2006/02,922,127, contents of which are herein incorporated by reference in their entirety. Brolen, G. K. et al., Diabetes (2005), 54:2867-2874 and Segev, H., Stem Cells (2004), 22:265-274, contents of which are herein incorporated by reference, describe methods for differentiation of human embryonic stem cells into β-cell like cells.

In some embodiments, the pancreatic cells are in a stabilized state, e.g., the cells were taken from a subject and treated in such a manner as to allow them to be stored for some period of time. For example, the cells can be frozen, e.g., using methods known in the art for freezing primary cells, such that the cells are viable when thawed. For example, methods known in the art to freeze and thaw embryos to generate live mammals can be adapted for use in the present methods. Such methods may include the use of liquid nitrogen, e.g., with one or more cryoprotectants, e.g., agents that prevent freeze-thaw damage to the cell.

The population of pancreatic cells obtained from a subject or donor can be substantially pure, e.g., not more than about 40% undifferentiated cells, i.e., at least about 60% fully differentiated pancreatic cells. In some embodiments, the population is at least about 70%, 75%, 80%, 90%, 95% or more fully-differentiated pancreatic cells. The purity of the population can be determined, and manipulated, using methods known in the art. For example, methods using fluorescence activated cell sorting can be used. For example, duct epithelial cells can be detected and counted, e.g., by labeling the cells with a fluorescence-labeled duct-specific lectin (e.g., Dolichos biflorus agglutinin (DBA)), as described herein, and removed from the population, e.g., by fluorescence-activated cell sorting methods (e.g., flow sorting) or immunosorbtion to a substrate, such as a column or beads, bound to DBA. Other non β-cells can be removed using similar methods, including flow sorting based on autofluorescence.

The population of pancreatic cells obtained from a subject can be homogeneous or heterogeneous. In some embodiments, the pancreatic cells obtained from a subject are of single cell type, e.g., alpha cell, beta cell, delta cell, or epsilon cell. In other embodiments, the pancreatic cells obtained from a subject comprise a mixture of different pancreatic cell types.

In some embodiments, the pancreatic cells are from a mammal, e.g., a mouse, a rat or a human. In some embodiments, the pancreatic cells are from a subject, where the subject is selected for based on subject's need of additional β-cells.

Contacting of Pancreatic Cells with Compounds

The pancreatic cell population can be contacted with the compounds described herein in a cell culture e.g., in vitro or ex vivo, or the compound can be administrated to a subject, e.g., in vivo. In some embodiments of the invention, a compound described herein can be administrated to a subject to treat, and/or prevent a disorder which is caused by a reduction in function and/or number of β-cells, e.g., hyperglycemia or diabetes.

The term “ex vivo” refers to cells which are removed from a living organism and cultured outside the organism (e.g., in a test tube).

The term “contacting” or “contact” as used herein in connection with contacting a population of pancreatic cells includes subjecting the pancreatic cells to an appropriate culture media which comprises the indicated compound or agent. Where the pancreatic cell population is in vivo, “contacting” or “contact” includes administering the compound or agent in a pharmaceutical composition to a subject via an appropriate administration route such that the compound or agent contacts the pancreatic cell population in vivo.

For in vivo methods, a therapeutically effective amount of a compound described herein can be administered to a subject. Methods of administering compounds to a subject are known in the art and easily available to one of skill in the art.

Promoting β-cell replication in a subject can lead to treatment, prevention or amelioration of a number of disorders which are caused by a reduction in function and/or number of β-cells, e.g., hyperglycemia or diabetes. Without wishing to be bound by theory, increasing β-cell replication in a subject leads to an increase in density and/or number of β-cells, e.g., β-cell mass.

As used herein, an increase in β-cell mass refers to an increase in number of β-cells, e.g. an increase in number of β-cells (e.g., pancreatic β-cells) in a subject being treated with a compound described herein as compared to the number of β-cells in the subject prior to the onset of treatment. The increase in β-cell mass can be at least 5%, 10%, 20%, 30%, 40%, 50%, 50%, 70%, 80%, 90%, 1-fold, 2-fold, 5-fold, 10-fold, 50-fold, 100-fold or more in treated subject compared to the β-cell mass in the subject prior to onset of treatment.

Pancreatic cells suitable for use in ex vivo methods can be prepared from a pancreas according to methods well known to those skilled in the art. For example, the harvested pancreas can be incubated with an enzyme solution at or about 37° C. to digest the pancreatic tissue into small clusters of tissue and cells. Following the appropriate digestion time the tissue digest can be filtered to remove large undigested tissue. The digested tissue may then can be applied to a density gradient such as Ficoll, polysucrose, dextran, and the like. The density gradient can either be continuous or discontinuous. The tissue loaded density gradient can then be centrifuged, and the cells contained within the digest migrate within the gradient according to their density. The cells can be retrieved from the gradient, washed, and placed in culture. Pancreatic cells prepared in this manner can contain multiple cell types.

For ex vivo methods, pancreatic cells can include autologous pancreatic cells, i.e., a cell or cells taken from a subject who is in need of additional β-cells (i.e., the donor and recipient are the same individual). Autologous pancreatic cells have the advantage of avoiding any immunologically-based rejection of the cells. Alternatively, the cells can be heterologous, e.g., taken from a donor. The second subject can be of the same or different species. Typically, when the cells come from a donor, they will be from a donor who is sufficiently immunologically compatible with the recipient, i.e., will not be subject to transplant rejection, to lessen or remove the need for immunosuppression. In some embodiments, the cells are taken from a xenogeneic source, i.e., a non-human mammal that has been genetically engineered to be sufficiently immunologically compatible with the recipient, or the recipient's species. Methods for determining immunological compatibility are known in the art, and include tissue typing to assess donor-recipient compatibility for HLA and ABO determinants. See, e.g., Transplantation Immunology, Bach and Auchincloss, Eds. (Wiley, John & Sons, Incorporated 1994). In some embodiments, pancreatic cells are recombinant β-cells, for example those described in U.S. Pat. Nos. 6,114,599; 6,242,254; and 6,448,045, contents of which are herein incorporated by reference in their entirety.

In some embodiments, the subject suffers from Type I, Type 1.5 or Type 2 diabetes or has a pre-diabetic condition.

Without wishing to be bound by theory any suitable cell culture media can be used for ex vivo methods of the invention. In some embodiments, the β-cells are cultured in the presence of a cell matrix protein, which protein is capable of promoting hemidesmosome formation. For example, the cell matrix proteins produced by the rat bladed carcinoma cell lines 804G or NBT-II are known in the art to promote hemidesmosome formation. Accordingly, U.S. Pat. No. 5,510,263, contents of which are herein incorporated by reference in their entirety, discloses the enhanced growth of pancreatic islet cells cultured on the 804G and NBT-II matrices.

In some embodiments, the cells are cultured in conditioned media from rat bladder carcinoma cell line 804G or NBT-II. The cells can also be cultured in media to which one or more of the matrix proteins from the conditioned media have been added. Such matrix proteins can be purified from natural sources or produced using recombinant methods known in the art.

In some other embodiments, the cells are cultured in culture media in contact with laminin 5. Preferably, the laminin 5 is selected from the group consisting of Kalinin and epiligrin. Laminin 5 can be obtained from a number of sources including, but not limited to, from the extracellular matrix obtained from MCF 10A cells.

After ex vivo contact with a compound described herein, when the pancreatic cells, e.g., β-cells have reached a desired population number or density, e.g., about 1×10⁶, 2×10⁶, 3×10⁶, 4×10⁶, 5×10⁶, 6×10⁶, 7×10⁶, 8×10⁶, 9×10⁶, 1×10⁷, 2×10⁷, or more cells, the cells can be transplanted in a subject who is in need of additional β-cells. The cells can be transplanted in a subject from whom the cells were originally obtained or in different subject. Methods for surgically removing and transplanting suitable pancreatic cells, e.g., beta-cells, from a mammal are known in the art; see, e.g., Shapiro et al., N. Engl. J. Med. 343(4):230-8 (2000); Ryan et al., Diabetes 50(4):710-9 (2001).

When the pancreatic cells are contacted with a compound, the compound can have a direct or an indirect affect on beta cells. As used herein, a “direct affect” means that the compound is directly interacting with the beta cells, e.g., binding to a cell surface receptor on the beta cell, taken up into the beta cells. As used herein, an “indirect affect” means that the compound does not directly interacts with the beta cell. For example, the compound can interact with a non-beta cell and indirectly influence the rate of beta cell replication or growth. Without wishing to be bound by theory, the compound can indirectly influence a beta cell by inducing expression and/or secretion of a molecule from a non beta cell, and this molecule then directly or indirectly influencing the rate of beta cell replication or growth.

### Methods of Monitoring Increase in β-Cell Replication

For ex vivo methods of the invention, increased β-cell replication can be monitored by any method known in the art for measuring cell replication. For example, β-cell replication can be determined by measuring the expression of at least one cell replication marker, e.g., Ki-67 or PH3. A non-limiting example is the quantitative immunofluorescent assay that measures mitotic index by monitoring histone H3 phosphorylation on serine 10 (H3-P), a mitosis-specific event (Ajiro et al., J Biol. Chem. 271:13197-201. 1996; Goto et al, J Biol Chem. 274:25543-9, 1999). Increase in β-cell replication can also be based on an increase in the total number of β-cells in the treated versus untreated control. In some instances, increased β-cell replication can be based on the ratio of β-cells to total cells for the treated and untreated controls. B-cell replication can be measured by monitoring the number of cells co-expressing Ki-67 and/or PH3, and PDX-1.

For in vivo methods of the invention, increased β-cell replication can be evaluated indirectly by measuring blood insulin levels. Without wishing to be bound by theory, blood insulin level is an indirect measure of the number of β-cells, e.g., β-cell mass in the subject. Therefore, blood insulin levels before and after onset of treatment can indirectly provide a relative measure of number of β-cells in the subject before and after onset of treatment. β-cell mass in a subject can also be determined by measuring the fasting blood glucose concentration in the subject. A curvilinear relationship between β-cell mass and fasting blood glucose concentrations in humans is disclosed in Ritzel, et. al., Diabetes Care (2006), 29:717-718, contents of which are herein incorporated by reference in their entirety. Alternatively, in vivo uptake of radioligand [11C]DTBZ (dihydrotetrabenazine), which specifically binds to VMAT2, by β-cells can be measured by positron emission tomography (P.E.T.) scanning. This radioligand has been used previously in human subjects in clinical trials evaluating P.E.T scanning of the brain in patients with bipolar illness and schizophrenia compared to healthy control subjects. U.S. Pat. Pub. No. 2009/0202428 describes use of DTBZ for imaging endocrine pancreas β-cell mass in type 1 diabetes, contents of which are herein incorporated by reference in theory entirety.

Methods for estimating in vivo β-cell mass are also described in, for example, Antkowiak, P. F., et al., Noninvasive assessment of pancreatic-beta-cell function in vivo with manganese-enhanced magnetic resonance imaging. Am J Physiol Endocrinol Metab (2009), 296:E573-E5788; Bergman, R. N., et al., Quantitative estimation of insulin sensitivity. Am J Physiol (1979), 236: E667-E677; Brunzell J. D., et al., Relationships between fasting plasma glucose levels and insulin secretion during intravenous glucose tolerance tests. J. Clin. Endocrinol. Metab (1976), 42:222-229; DeFronzo, R. A., et al., Glucose clamp technique: a method for quantifying insulin secretion and resistance. Am J Physiol (1979), 237: E214-E223; Evgenov N. V., et al., In vivo imaging of islet transplantation. Nat Med (2006), 12:144-148; Kjems, L. L., et al., Decrease in beta-cell mass leads to impaired pulsatile insulin secretion, reduced postprandial hepatic insulin clearance, and relative hyperglucagonemia in the minipig. Diabetes (2001), 50: 2001-2012; Larsen, M. O., et al., Loss of beta-cell mass leads to a reduction of pulse mass with normal periodicity, regularity and entrainment of pulsatile insulin secretion in Göttingen minipigs. Diabetologia (2003), 46: 195-202; Larsen, M. O., et al., Measurements of insulin secretory capacity and glucose tolerance to predict pancreatic beta-cell mass in vivo in the nicotinamide/streptozotocin Göttingen minipig, a model of moderate insulin deficiency and diabetes. Diabetes (2003), 52: 118-123; Larsen, M. O. et al., Measures of Insulin Responses as Predictive Markers of Pancreatic Beta-Cell Mass in Normal and Bet-Cell Reduced Lean and Obese Göttingen minipigs in vivo. Am J Physiol Endocrinol Metab (2005), 2006, 290: E670-E677; McCulloch, D. K., et al., Correlations of in vivo beta-cell function tests with beta-cell mass and pancreatic insulin content in streptozocin-administered baboons. Diabetes (1991), 40: 673-679; Meier, J. J., et al. Functional Assessment of Pancreatic {beta}-Cell Area in Humans. Diabetes, (2009), 58: 1595-1603; Souza F, et al., Longitudinal noninvasive PET-based beta cell mass estimates in a spontaneous diabetes rat model. J. Clin. Invest. (2006), 116: 1506-1513; Tobin B. W., et al., Insulin secretory function in relation to transplanted islet mass in STZ-induced diabetic rats. Diabetes (1993), 42:98-105; and Ward, W. K., et al., Diminished B cell secretory capacity in patients with noninsulin dependent diabetes mellitus. J Clin Invest (1984), 74: 1318-1328, contents of which are herein incorporated by reference in their entirety.

### Treatment of Diabetes

The methods described herein are useful in treating disorders associated with a loss of β-cells, e.g., hyperglycemia or diabetes. The methods can include administering a ADK inhibitor, SAHH inhibitor, and/or AMPK activator to the subject. The compounds can be administered systemically or locally, e.g., by injection or implantation of a device that provides a steady dose of the compound to the pancreatic tissues, e.g., to the islets. Such devices are known in the art, and include micro-pumps and controlled-release matrices, e.g., matrices that break down over time, releasing the modulator into the tissue.

Alternatively, the methods include cell-based therapies. For example, The methods can include implanting into a subject a population of β-cells that has been expanded or increased by a method described herein. In some embodiments, the cells are autologous, e.g., they come from the same subject into which they will be transplanted. Surgical methods for implanting such cells are known in the art, and include minimally-invasive, endoscopic methods. Generally, for humans, it is desirable to implant at least about a mean (±SD) islet mass of 10,000 islet equivalents per kilogram of body weight, see, e.g., Shapiro et al, N. Engl. J. Med. 343(4):230-8 (2000).

In one aspect, the invention provides for a method of increasing β-cell mass or insulin production in a subject, the method comprising: (a) contacting a β-cell with an ADK inhibitor, SAHH inhibitor, or AMPK activator, in a cell culture; (b) allowing the cell to replicate for a time sufficient to produce a desired number or density of cells; and (c) introducing the cells from step (b) into a subject.

In some embodiments, the method comprises the additional step of obtaining β-cells from a subject.

In some embodiments, cells are allowed to replicate for a sufficient time such that there about 1×10⁶, 2×10⁶, 3×10⁶, 4×10⁶, 5×10⁶, 6×10⁶, 7×10⁶, 8×10⁶, 9×10⁶, 1×10⁷, 2×10⁷, or more cells, in the cell culture.

The methods by which such cells can be introduced into the subject are described herein. One representative method involves the encapsulation of cells in a biocompatible coating. In this approach, cells are entrapped in a capsular coating that protects the encapsulated cells from immunological responses, and also serves to prevent uncontrolled proliferation and spread of the cells. An exemplary encapsulation technique involves encapsulation with alginate-polylysine-alginate. In particular embodiments, capsules made by employing this technique generally contain several hundred cells and have a diameter of approximately 1 mm.

Cells can be implanted using the alginate-polylysine encapsulation-technique of O'Shea and Sun (1986), Diabetes 35:943, with modifications as described by Fritschy et al. (1991) Diabetes 40:37. According to this method, the cells are suspended in 1.3% sodium alginate and encapsulated by extrusion of drops of the cell/alginate suspension through a syringe into CaCl2. After several washing steps, the droplets are suspended in polylysine and rewashed. The alginate within the capsules is then reliquified by suspension in 1 ml EGTA and then rewashed with Krebs balanced salt buffer. Each capsule should contain several hundred cells and have a diameter of approximately one mm.

Implantation of encapsulated islets into animal models of diabetes by the above method has been shown to significantly increase the period of normal glycemic control, by prolonging xenograft survival compared to unencapsulated islets (O'Shea and Sun (1986), Diabetes 35:943; Fritschy, et al. (1991) Diabetes 40:37). Also, encapsulation can prevent uncontrolled proliferation of clonal cells. Capsules containing cells can be implanted (e.g., from about 500, 1,000 or 2,000 cells to about 5,000, 10,000 or 20,000 cells/animal) intraperitoneally and blood samples taken daily for monitoring of blood glucose and insulin.

An alternative approach is to seed Amicon fibers with cells. The cells become enmeshed in the fibers, which are semipermeable, and are thus protected in a manner similar to the micro encapsulates (Altman et al., (1986) Diabetes 35:625).

After successful encapsulation or fiber seeding, the cells, generally approximately 1,000-10,000, can be implanted intraperitoneally, usually by injection into the peritoneal cavity through a large gauge needle (23 gauge).

A variety of other encapsulation technologies have been developed that are applicable to the practice of the present invention (see, e.g., Lacy et al., (1991), Science, 254:1782-1784; Sullivan et al. Science, 252:718-721; PCT publications WO 91/10470; WO 91/10425; WO 90/15637; WO 90/02580; WO 8901967; U.S. Pat. No. 5,011,472; U.S. Pat. No. 4,892,538; contents of which are herein incorporated by reference in their entirety. The company Cyto Therapeutics has developed encapsulation technologies that are now commercially available and are of use in the application of the present invention. A vascular device has also been developed by Biohybrid, of Shrewsbury, Mass, which has application to the technology of the present invention.

With respect to implantation methods, particular advantages can be found in the methods recently described by Lacy et al. (1991), Science, 254:1782-1784, and Sullivan et al, (1991) Science, 252:718-721, each incorporated herein by reference in its entirety for teachings of implantation methods. These concern, firstly, the subcutaneous xenograft of encapsulated islets, and secondly, the long-term implantation of islet tissue in an “artificial pancreas” which can be connected to the vascular system as an arteriovenous shunt. These implantation methods can be advantageously adapted for use with the present invention by employing the expanded cells, as disclosed herein, in the place of the “islet tissue” described in these publications.

Lacy et al. ((1991), Science, 254:1782-1784) describes the encapsulation of rat islets in hollow acrylic fibers and immobilization of these in alginate hydrogel. Following intraperitoneal transplantation of the encapsulated islets into diabetic mice, normoglycemia was reportedly restored. Similar results were also obtained using subcutaneous implants that had an appropriately constructed outer surface on the fibers. The expanded cells of the present invention can also be straightforwardly “transplanted” into a mammal by similar subcutaneous injection.

A biohybrid perfused “artificial pancreas,” which encapsulates islet tissue in a selectively permeable membrane, can also be employed (Sullivan et al, (1991) Science, 252:718-721). In this embodiment, a tubular semi-permeable membrane is coiled inside a protecting housing to provide a compartment for the islet cells. Each end of the membrane is then connected to an arterial polytetrafluoroethylene (PTFE) graft that extends beyond the housing and joins the device to the vascular system as an arteriovenous shunt. The implantation of such a device containing islet allografts into pancreatectomized dogs was reported to result in the control of fasting glucose levels. Grafts of this type encapsulating modified cells described herein can also be used in accordance with the present invention.

An alternate approach to encapsulation is to simply inject the cells into the scapular region or peritoneal cavity of diabetic mice or rats, where these cells are reported to form tumors (Sato et al, (1962) Proc. Natl. Acad. Sci. USA 48:1184).

### Pharmaceutical Compositions

For administration to a subject, the compounds can be provided in pharmaceutically acceptable compositions. These pharmaceutically acceptable compositions comprise a therapeutically-effective amount of one or more of the ADK inhibitors and AMPK activators described above, formulated together with one or more pharmaceutically acceptable carriers (additives) and/or diluents. As described in detail below, the pharmaceutical compositions of the present invention can be specially formulated for administration in solid or liquid form, including those adapted for the following: (1) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally, compounds can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

The phrase “therapeutically-effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment. For example, an amount of a compound administered to a subject that is sufficient to produce a statistically significant, measurable change in at least one symptom of Type 1, Type 1.5 or Type 2 diabetes, such as glycosylated hemoglobin level, fasting blood glucose level, hypoinsulinemia, etc. . . . . Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

As used herein, the term “administer” refers to the placement of a composition into a subject by a method or route which results in at least partial localization of the composition at a desired site such that desired effect is produced. A compound or composition described herein can be administered by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In preferred embodiments, the compositions are administered by intravenous infusion or injection.

By “treatment”, “prevention” or “amelioration” of a disease or disorder is meant delaying or preventing the onset of such a disease or disorder, reversing, alleviating, ameliorating, inhibiting, slowing down or stopping the progression, aggravation or deterioration the progression or severity of a condition associated with such a disease or disorder. In one embodiment, the symptoms of a disease or disorder are alleviated by at least 5%, at least 10%, at least 20%, at least 30%, at least 40%, or at least 50%.

Treatment of Diabetes is determined by standard medical methods. A goal of Diabetes treatment is to bring sugar levels down to as close to normal as is safely possible. Commonly set goals are 80-120 milligrams per deciliter (mg/dl) before meals and 100-140 mg/dl at bedtime. A particular physician may set different targets for the patent, depending on other factors, such as how often the patient has low blood sugar reactions. Useful medical tests include tests on the patient's blood and urine to determine blood sugar level, tests for glycosylated hemoglobin level (HbA1c; a measure of average blood glucose levels over the past 2-3 months, normal range being 4-6%), tests for cholesterol and fat levels, and tests for urine protein level. Such tests are standard tests known to those of skill in the art (see, for example, American Diabetes Association, 1998). A successful treatment program can also be determined by having fewer patients in the program with complications relating to Diabetes, such as diseases of the eye, kidney disease, or nerve disease.

Delaying the onset of diabetes in a subject refers to delay of onset of at least one symptom of diabetes, e.g., hyperglycemia, hypoinsulinemia, diabetic retinopathy, diabetic nephropathy, blindness, memory loss, renal failure, cardiovascular disease (including coronary artery disease, peripheral artery disease, cerebrovascular disease, atherosclerosis, and hypertension), neuropathy, autonomic dysfunction, hyperglycemic hyperosmolar coma, or combinations thereof, for at least 1 week, at least 2 weeks, at least 1 month, at least 2 months, at least 6 months, at least 1 year, at least 2 years, at least 5 years, at least 10 years, at least 20 years, at least 30 years, at least 40 years or more, and can include the entire lifespan of the subject.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments, the subject is a mammal, e.g., a primate, e.g., a human. The terms, “patient” and “subject” are used interchangeably herein. The terms, “patient” and “subject” are used interchangeably herein.

Preferably, the subject is a mammal. The mammal can be a human, non-human primate, mouse, rat, dog, cat, horse, or cow, but are not limited to these examples. Mammals other than humans can be advantageously used as subjects that represent animal models of Type 1 diabetes, Type 2 Diabetes Mellitus, or pre-diabetic conditions. In addition, the methods described herein can be used to treat domesticated animals and/or pets. A subject can be male or female. A subject can be one who has been previously diagnosed with or identified as suffering from or having Diabetes (e.g., Type 1 or Type 2), one or more complications related to Diabetes, or a pre-diabetic condition, and optionally, but need not have already undergone treatment for the Diabetes, the one or more complications related to Diabetes, or the pre-diabetic condition. A subject can also be one who is not suffering from Diabetes or a pre-diabetic condition. A subject can also be one who has been diagnosed with or identified as suffering from Diabetes, one or more complications related to Diabetes, or a pre-diabetic condition, but who show improvements in known Diabetes risk factors as a result of receiving one or more treatments for Diabetes, one or more complications related to Diabetes, or the pre-diabetic condition. Alternatively, a subject can also be one who has not been previously diagnosed as having Diabetes, one or more complications related to Diabetes, or a pre-diabetic condition. For example, a subject can be one who exhibits one or more risk factors for Diabetes, complications related to Diabetes, or a pre-diabetic condition, or a subject who does not exhibit Diabetes risk factors, or a subject who is asymptomatic for Diabetes, one or more Diabetes-related complications, or a pre-diabetic condition. A subject can also be one who is suffering from or at risk of developing Diabetes or a pre-diabetic condition. A subject can also be one who has been diagnosed with or identified as having one or more complications related to Diabetes or a pre-diabetic condition as defined herein, or alternatively, a subject can be one who has not been previously diagnosed with or identified as having one or more complications related to Diabetes or a pre-diabetic condition.

As used herein, the phrase “subject in need of additional β-cells” refers to a subject who is diagnosed with or identified as suffering from, having or at risk for developing diabetes (e.g., Type 1, Type 1.5 or Type 2), one or more complications related to diabetes, or a pre-diabetic condition.

A subject in need of additional β-cells can be identified using any method used for diagnosis of diabetes. For example, Type 1 diabetes can be diagnosed using a glycosylated hemoglobin (A1C) test, a random blood glucose teat and/or a fasting blood glucose test. Parameters for diagnosis of diabetes are known in the art and available to skilled artisan without much effort.

In some embodiments, the methods of the invention further comprise selecting a subject identified as being in need of additional β-cells. A subject in need of additional β-cells can be selected based on the symptoms presented, such as symptoms of type 1, type 1.5 or type 2 diabetes. Exemplary symptoms of diabetes include, but are not limited to, excessive thirst (polydipsia), frequent urination (polyuria), extreme hunger (polyphagia), extreme fatigue, weight loss, hyperglycemia, low levels of insulin, high blood sugar (e.g., sugar levels over 250 mg, over 300 mg), presence of ketones present in urine, fatigue, dry and/or itchy skin, blurred vision, slow healing cuts or sores, more infections than usual, numbness and tingling in feet, diabetic retinopathy, diabetic nephropathy, blindness, memory loss, renal failure, cardiovascular disease (including coronary artery disease, peripheral artery disease, cerebrovascular disease, atherosclerosis, and hypertension), neuropathy, autonomic dysfunction, hyperglycemic hyperosmolar coma, and combinations thereof.

The ADK inhibitor, SAHH inhibitor, and/or AMPK activator can be administrated to a subject in combination with a pharmaceutically active agent. Exemplary pharmaceutically active compound include, but are not limited to, those found in Harrison's Principles of Internal Medicine, 13^{th} Edition, Eds. T. R. Harrison et al. McGraw-Hill N.Y., NY; Physicians Desk Reference, 50^{th} Edition, 1997, Oradell New Jersey, Medical Economics Co.; Pharmacological Basis of Therapeutics, 8^{th} Edition, Goodman and Gilman, 1990; United States Pharmacopeia, The National Formulary, USP XII NF XVII, 1990; current edition of Goodman and Oilman's The Pharmacological Basis of Therapeutics; and current edition of The Merck Index, the complete contents of all of which are incorporated herein by reference. In some embodiments, pharmaceutically active agent include those agents known in the art for treatment of diabetes and or for having anti-hyperglycemic activities, for example, inhibitors of dipeptidyl peptidase 4 (DPP-4) (e.g., AlogIiptin, Linagliptin, Saxagliptin, Sitagliptin, Vildagliptin, and Berberine), biguanides (e.g., Metformin, Buformin and Phenformin), peroxisome proliferator-activated receptor (PPAR) modulators such as thiazolidinediones (TZDs) (e.g., Pioglitazone, Rivoglitazone, Rosiglitazone and Troglitazone), dual PPAR agonists (e.g., Aleglitazar, Muraglitazar and Tesaglitazar), sulfonylureas (e.g., Acetohexamide, Carbutamide, Chlorpropamide, Gliclazide, Tolbutamide, Tolazamide, Glibenclamide (Glyburide), Glipizide, Gliquidone, Glyclopyramide, and Glimepiride), meglitinides (“glinides”) (e.g., Nateglinide, Repaglinide and Mitiglinide), glucagon-like peptide-1 (GLP-1) and analogs (e.g., Exendin-4, Exenatide, Liraglutide, Albiglutide), insulin and insulin analogs (e.g., Insulin lispro, Insulin aspart, Insluin glulisine, Insulin glargine, Insulin detemir, Exubera and NPH insulin), alpha-glucosidase inhibitors (e.g., Acarbose, Miglitol and Voglibose), amylin analogs (e.g. Pramlintide), Sodium-dependent glucose cotransporter T2 (SGLT T2) inhibitors (e.g., Dapgliflozin, Remogliflozin and Sergliflozin) and others (e.g. Benfluorex and Tolrestat).

In type 1 diabetes, β-cells are undesirably destroyed by continued autoimmune response. This autoimmune response can be attenuated by use of compounds that inhibit or block such an autoimmune response. This can reduce the length of treatment regime needed to establish the needed and/or required β-cell mass levels. In some embodiments, the pharmaceutically active agent is a immune response modulator. As used herein, the term “immune response modulator” refers to compound (e.g., a small-molecule, antibody, peptide, nucleic acid, or gene therapy reagent) that inhibits autoimmune response in a subject. Without wishing to be bound by theory, an immune response modulator inhibits the autoimmune response by inhibiting the activity, activation, or expression of inflammatory cytokines (e.g., IL-12, IL-23 or IL-27), or STAT-4. Exemplary immune response modulators include, bbut are not limited to, members of the group consisting of Lisofylline (LSF) and the LSF analogs and derivatives described in U.S. Pat. No. 6,774,130, contents of which are herein incorporated by reference in their entirety.

The ADK inhibitor, SAHH inhibitor, and/or the AMPK activator and the pharmaceutically active agent can be administrated to the subject in the same pharmaceutical composition or in different pharmaceutical compositions (at the same time or at different times). When administrated at different times, compound of the invention and the pharmaceutically active agent can be administered within 5 minutes, 10 minutes, 20 minutes, 60 minutes, 2 hours, 3 hours, 4, hours, 8 hours, 12 hours, 24 hours of administration of the other When ADK inhibitor, SAHH inhibitor or the AMPK activator, and the pharmaceutically active agent are administered in different pharmaceutical compositions, routes of administration can be different. For example, an ADK inhibitor, SAHH inhibitor or AMPK activator is administered by any appropriate route known in the art including, but not limited to oral or parenteral routes, including intravenous, intramuscular, subcutaneous, transdermal, airway (aerosol), pulmonary, nasal, rectal, and topical (including buccal and sublingual) administration, and pharmaceutically active agent is administration by a different route, e.g. a route commonly used in the art for administration of said pharmaceutically active agent. In a non-limiting example, an ADK inhibitor of formula (II) (e.g., B8) can be administered orally, while a pharmaceutically active agent (e.g., DPP-4 inhibitor) can be administrated subcutaneously.

The amount of compound which can be combined with a carrier material to produce a single dosage form will generally be that amount of the compound which produces a therapeutic effect. Generally out of one hundred percent, this amount will range from about 0.1% to 99% of compound, preferably from about 5% to about 70%, most preferably from 10% to about 30%.

Toxicity and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compositions that exhibit large therapeutic indices, are preferred.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized.

The therapeutically effective dose can be estimated initially from cell culture assays. A dose may be formulated in animal models to achieve a circulating plasma concentration range that includes the IC50 (i.e., the concentration of the therapeutic which achieves a half-maximal inhibition of symptoms) as determined in cell culture. Levels in plasma may be measured, for example, by high performance liquid chromatography. The effects of any particular dosage can be monitored by a suitable bioassay.

The dosage may be determined by a physician and adjusted, as necessary, to suit observed effects of the treatment. Generally, the compositions are administered so that ADK inhibitor, SAHH inhibitor, and/or the AMPK activator is given at a dose from 1 μg/kg to 150 mg/kg, 1 μg/kg to 100 mg/kg, 1 μg/kg to 50 mg/kg, 1 μg/kg to 20 mg/kg, 1 μg/kg to 10 mg/kg, 1 μg/kg to 1 mg/kg, 100 μg/kg to 100 mg/kg, 100 μg/kg to 50 mg/kg, 100 μg/kg to 20 mg/kg, 100 μg/kg to 10 mg/kg, 100 μg/kg to 1 mg/kg, 1 mg/kg to 100 mg/kg, 1 mg/kg to 50 mg/kg, 1 mg/kg to 20 mg/kg, 1 mg/kg to 10 mg/kg, 10 mg/kg to 100 mg/kg, 10 mg/kg to 50 mg/kg, or 10 mg/kg to 20 mg/kg. It is to be understood that ranges given here include all intermediate ranges, for example, the range 1 tmg/kg to 10 mg/kg includes 1 mg/kg to 2 mg/kg, 1 mg/kg to 3 mg/kg, 1 mg/kg to 4 mg/kg, 1 mg/kg to 5 mg/kg, 1 mg/kg to 6 mg/kg, 1 mg/kg to 7 mg/kg, 1 mg/kg to 8 mg/kg, 1 mg/kg to 9 mg/kg, 2 mg/kg to 10 mg/kg, 3 mg/kg to 10 mg/kg, 4 mg/kg to 10 mg/kg, 5 mg/kg to 10 mg/kg, 6 mg/kg to 10 mg/kg, 7 mg/kg to 10 mg/kg, 8 mg/kg to 10 mg/kg, 9 mg/kg to 10 mg/kg etc. . . . . It is to be further understood that the ranges intermediate to the given above are also within the scope of this invention, for example, in the range 1 mg/kg to 10 mg/kg, dose ranges such as 2 mg/kg to 8 mg/kg, 3 mg/kg to 7 mg/kg, 4 mg/kg to 6 mg/kg etc.

With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment or make other alteration to treatment regimen. The dosing schedule can vary from once a week to daily depending on a number of clinical factors, such as the subject's sensitivity to the polypeptides. The desired dose can be administered at one time or divided into subdoses, e.g., 2-4 subdoses and administered over a period of time, e.g., at appropriate intervals through the day or other appropriate schedule. Such sub-doses can be administered as unit dosage forms. In some embodiments, administration is chronic, e.g., one or more doses daily over a period of weeks or months. Examples of dosing schedules are administration daily, twice daily, three times daily or four or more times daily over a period of 1 week, 2 weeks, 3 weeks, 4 weeks, 1 month, 2 months, 3 months, 4 months, 5 months, or 6 months or more.

### Screening Assays

In yet another aspect, the invention provides for a method of screening for a candidate compound for stimulating or increasing replication or growth of a β-cell in a pancreatic cell population, the method comprising:

(a) contacting a population of pancreatic cells with a test compound;

(b) assessing beta-cell replication; and

(c) selecting the compound that increases or enhances β-cell replication.

The pancreatic cell population can comprise different types of pancreatic cells, including but not limited to, α-cells, β-cells, δ-cells, and fibroblasts. Accordingly, in some embodiments, at least 10%, a least 20%, at least 30%, at least 40%, at least 50%, at least 60%, at least 70%, at least 80%, at least 90%, or at least 95% of the cells in the pancreatic cell population are β-cells.

In some embodiments, 90% or less, 80% or less, 70% or less, 60% or less, 50% or less, 40% or less, 30% or less, 20% or less, 10% or less, or 5% or less of the cells in the pancreatic cell population are α-cells.

In some embodiments, 90% or less, 80% or less, 70% or less, 60% or less, 50% or less, 40% or less, 30% or less, 20% or less, 10% or less, or 5% or less of the cells in the pancreatic cell population are δ-cells.

In some embodiments, 90% or less, 80% or less, 70% or less, 60% or less, 50% or less, 40% or less, 30% or less, 20% or less, 10% or less, or 5% or less of the cells in the pancreatic cell population are fibroblasts.

In some embodiments, the pancreatic cell population comprises 50-90% β-cells, 10-30% α-cells, 5-10% fibroblasts, and 5-10% other cell types. In one further embodiment of this the pancreatic cell population comprises about 75% β-cells, about 18% α-cells, about 3% fibroblasts, and about 5% other cell types.

As used herein, the term “test compound” refers to compounds and/or compositions that are to be screened for their ability to stimulate and/or increase β-cell replication and/or growth. The test compounds can include a wide variety of different compounds, including chemical compounds and mixtures of chemical compounds, e.g., small organic or inorganic molecules; saccharines; oligosaccharides; polysaccharides; biological macromolecules, e.g., peptides, proteins, and peptide analogs and derivatives; peptidomimetics; nucleic acids; nucleic acid analogs and derivatives; an extract made from biological materials such as bacteria, plants, fungi, or animal cells; animal tissues; naturally occurring or synthetic compositions; and any combinations thereof. In some embodiments, the test compound is a small molecule.

As used herein, the term “small molecule” can refer to compounds that are “natural product-like,” however, the term “small molecule” is not limited to “natural product-like” compounds. Rather, a small molecule is typically characterized in that it contains several carbon-carbon bonds, and has a molecular weight of less than 5000 Daltons (5 kD), preferably less than 3 kD, still more preferably less than 2 kD, and most preferably less than 1 kD. In some cases it is preferred that a small molecule have a molecular weight equal to or less than 700 Daltons.

The number of possible test compounds runs into millions. Methods for developing small molecule, polymeric and genome based libraries are described, for example, in Ding, et al. J Am. Chem. Soc. 124: 1594-1596 (2002) and Lynn, et al., J. Am. Chem. Soc. 123: 8155-8156 (2001). Commercially available compound libraries can be obtained from, e.g., ArQule, Pharmacopia, graffinity, Panvera, Vitas-M Lab, Biomol International and Oxford. These libraries can be screened using the screening devices and methods described herein. Chemical compound libraries such as those from NIH Roadmap, Molecular Libraries Screening Centers Network (MLSCN) can also be used. A comprehensive list of compound libraries can be found at www.broad.harvard.edu/chembio/platform/screening/compound_libraries/index.htm. A chemical library or compound library is a collection of stored chemicals usually used ultimately in high-throughput screening or industrial manufacture. The chemical library can consist in simple terms of a series of stored chemicals. Each chemical has associated information stored in some kind of database with information such as the chemical structure, purity, quantity, and physiochemical characteristics of the compound.

Depending upon the particular embodiment being practiced, the test compounds can be provided free in solution, or may be attached to a carrier, or a solid support, e.g., beads. A number of suitable solid supports may be employed for immobilization of the test compounds. Examples of suitable solid supports include agarose, cellulose, dextran (commercially available as, i.e., Sephadex, Sepharose) carboxymethyl cellulose, polystyrene, polyethylene glycol (PEG), filter paper, nitrocellulose, ion exchange resins, plastic films, polyaminemethylvinylether maleic acid copolymer, glass beads, amino acid copolymer, ethylene-maleic acid copolymer, nylon, silk, etc. Additionally, for the methods described herein, test compounds may be screened individually, or in groups. Group screening is particularly useful where hit rates for effective test compounds are expected to be low such that one would not expect more than one positive result for a given group.

In some embodiments, the test compound increases beta-cell replication or growth by at least 5%, 10%, 20%, 30%, 40%, 50%, 50%, 70%, 80%, 90%, 1-fold, 1.1-fold, 1.5-fold, 2-fold, 3-fold, 4-fold, 5-fold, 10-fold, 50-fold, 100-fold or more higher relative to an untreated control.

In some embodiments, the step of assessing beta-cell replication comprises detecting a β-cell marker and a cell-replication marker. A selected test compound can be further limited to the compound where the β-cell marker and the cell-replication marker co-localize in the same cell.

Increased or enhanced β-cell replication can be assessed by: (i) increased total number of cells in the culture, as compared to an untreated control; (ii) increased total number of cells expressing at least one β-cell marker in the culture, as compared to an untreated control; (iii) increased ratio of cells expressing at least one β-cell marker to the total number of cells in the culture, as compared to an untreated control; (iv) increased number of cells expressing at least one cell-replication marker, as compared to an untreated control; (v) increased ratio of cells expressing at least one cell-replication marker, as compared to an untreated control; or (vi) a combination thereof.

In some embodiments, beta-cell replication is assessed via automated image acquisition and analysis using a Cellomics ArrayScan VTI. The acquisition thresholds/parameters are established such that the computer-based calls of replication events are consistent with human-based calls. Such automated image acquisition and analysis allows for high-throughput screening of compounds.

In some embodiments of this and other aspects of the invention, the pancreatic cells are cultured in the presence of cell matrix proteins capable of promoting hemidesmosome formation, such as those produced by the rat bladed carcinoma cell lines 804G or NBT-II. U.S. Pat. No. 5,510,263, contents of which are herein incorporated by reference in their entirety, discloses the enhanced growth of pancreatic islet cells cultured on the 804G and NBT-II matrices.

In some embodiments of this and other aspects of the invention, the cells are cultured in conditioned media from rat bladder carcinoma cell line 804G or NBT-II. The cells can also be cultured in media to which one or more of the matrix proteins from the conditioned media have been added. Such matrix proteins can be purified from natural sources or produced using recombinant methods known in the art.

In some embodiments of this and other aspects of the invention, the cells are cultured in culture media in contact with laminin 5. Preferably, the laminin 5 is selected from the group consisting of Kalinin and epiligrin. Laminin 5 can be obtained from a number of sources including, but not limited to, from the extracellular matrix obtained from MCF 10A cells.

It is to be understood that, the conditioned media or matrix proteins can be added to the culture media. Alternatively, the conditioned media or matrix proteins can be used to precoat the surface of the vessel where pancreatic cells are to be cultured. Preferably, surface of the vessel is coated with 804G or NBT-II conditioned media, before plating of the pancreatic cells.

Generally plating density can range from about 10 k cells/well to about 100 k cells/well. In some embodiments, cellular plating density is in the range from about 25 k cells/well to about 75 k cells/well. In one embodiment, cellular plating density is about 60 k cells/well. Generally, at least 75%, 80%, 85%, 90%, 95% or more of the cells are viable at time of plating.

After plating, pancreatic cells can be allowed to adhere to the surface for a sufficient time, e.g. at least at least 1 hour, 2 hours, 3, hours, 4 hours, 6 hours, 8 hours, 12 hours, 24 hours, 36 hours, 48 hours or more, before contacting with the test compound. In some embodiments, the cells are allowed to adhere for 48 hours before compound treatment. After the cells have been allowed to adhere for a sufficient time, the media can be changed before treatment with compound of interest.

Generally, compounds can be tested at any concentration that can enhance replication of β-cells relative to a control over an appropriate time period. In some embodiments, compounds are tested at concentration in the range of about 0.1 nM to about 1000 mM. Preferably the compound is tested in the range of about 0.1 μM to about 20 μM, about 0.1 μM to about 10 μM, or about 0.1 μM to about 5 μM. In one embodiment, compounds are tested at 1 μM.

The pancreatic cell population can be maintained at any temperature suitable for pancreatic cell cultures. In one embodiment, the pancreatic cells are maintained at a temperature in the range of about 15° C. to about 55° C. In one embodiment, the pancreatic cells are maintained at 37° C.

Generally, the number of β-cells in the culture can be counted after the pancreatic cells have been in contact with the test compound for a sufficient time, e.g., at least 1 hour, at least 2 hours, at least 3, hours, at least 4 hours, at least 5 hours, at least 6 hours, at least 7 hours, at least 8 hours, at least 9 hours, at least 10 hours, at least 11 hours, at least 12 hours, at least 24 hours, at least 36 hours, at least 48 hours, at least 5 days, at least 1 week, at least 2 weeks, at least 3 weeks, or more. The cells can be counted manually or by an automated system. Use of an automated system allows for high-throughput screening of compounds.

Beta-cell and replication cell marker detection can be done after the pancreatic cells are in contact with the test compound for a sufficient time, e.g., at least 1 hour, at least 2 hours, at least 3, hours, at least 4 hours, at least 5 hours, at least 6 hours, at least 7 hours, at least 8 hours, at least 9 hours, at least 10 hours, at least 11 hours, at least 12 hours, at least 24 hours, at least 36 hours, at least 48 hours, at least 5 days, at least 1 week, at least 2 weeks, at least 3 weeks, or more. After marker detection, number of cells expressing cell-replication and/or β-cell marker can be counted. Marker detection can include the steps of preparing the cells for the appropriate assay, e.g., fixing and/or staining the cells.

In some embodiments, the method comprises additionally selecting the compound that increased the ratio of β-cells to the total number of cells as compared to an untreated control.

The term “β-cell marker” refers to, without limitation, proteins, peptides, nucleic acids, polymorphism of proteins and nucleic acids, splice variants, fragments of proteins or nucleic acids, elements, and other analytes which are specifically expressed or present in β-cells. Exemplary β-cell markers include, but are not limited to, pancreatic and duodenal homeobox 1 (PDX-1) polypeptide, insulin, c-peptide, amylin, E-cadherin, Hnf3β, PCI/3, Beta2, Nkx2.2, Nkx6.1, GLUT2, PC2, ZnT-8, MAFA, MAFB, and those described in Zhang et al., Diabetes. 50(10):2231-6 (2001). In some embodiment, the β-cell marker is a nuclear β-cell marker. In some embodiments, the β-cell marker is PDX-1 or PH3.

Without wishing to be bound by theory, the failure of prior β-cell replication screens is primarily a consequence of using a cytoplasmic marker (Insulin) as the β-cell identifier.

The use of a cytoplasmic marker prevents accurate attribution of nuclear replication to a specific cell identity, i.e., in dense culture it is impossible to attribute a cytoplasm to a specific nucleus given the proximity to multiple nuclei. Therefore, in some embodiments, β-cell marker is not a cytoplasmic β-cell marker. In one embodiment, β-cell marker is not insulin.

The terms “cell replication marker” refers to, without limitation, proteins, peptides, nucleic acids, polymorphism of proteins and nucleic acids, splice variants, fragments of proteins or nucleic acids, elements, and other analytes which are specifically associated with cell proliferation. Additionally, “cell-replication marker”, includes enzymatic activity when changes, e.g., increase or decrease, in the enzymatic activity are specifically associated with cell proliferation. Exemplary cell replication markers include, but are not limited to, phosphorylated histone H3 (PH3), Ki-67 protein, phosphorylated MPM-2 antigen, Proliferating Cell Nuclear Antigen (PCNA, a protein that is expressed in the nuclei of cells during the DNA synthesis phase of the cell cycle), phospho-S780-Rb epitope (Jacobberger, J W, et al. Cytometry A (2007), 73A:5-15). Cenp-F (mitosin), class III β-Tublin, spindal checkpoint protine hMad2, phosphorylated myosin light chain kinase, topoisomerase II, Check point kinase 1 (Chk1), Vesicular Monoamine Transporter 2 (VMAT2), loss of cyclin-dependent kinase 1 (Cdk1) kinase activity. Histone H3 can be phosphorylated at Ser28 or Ser10.

In some embodiments, cell replication marker is Ki-67 protein or PH3.

The Ki-67 protein (also known as MKI67) is a cellular marker for proliferation. It is strictly associated with cell proliferation. During interphase, the Ki-67 protein can be exclusively detected within the cell nucleus, whereas in mitosis most of the protein is relocated to the surface of the chromosomes. Ki-67 protein is present during all active phases of the cell cycle (G₁, S, G₂, and mitosis), but is absent from resting cells (G₀). Ki-67 is an excellent marker to determine the growth fraction of a given cell population.

Cell replication markers and β-cell markers can be detected by methods known in the art and easily available to the skilled artisan, for example appropriate ELISA, immunofluorescent, or immunohistochemical assays can be used for detection. MIB-1 is a commonly used monoclonal antibody that detects the Ki-67 protein. It is used in clinical applications to determine the Ki-67 labelling index. Ki-67 ELISA are described in Klein, C L, et al., J. Mater. Sci. Mater. Med. (2000), 11:125-132; Frahm, S O, et al., J. Immunol. Methods (199*0, 211:43-50; and Key G, et al., J. Immunol. Methods (1994), 177:113-117. Phospho-Histone H3 antibodies for detection of phosphorylated Histone H3 are commercially available from Cell Signaling Technology and Millipore. Antibodies against PCNA are commercially available from Sigma Aldrich. Antibodies to MPM-2 antigen are specific for cells in mitosis, recognizes a family of proteins that share a common phosphorylated epitope.

In some embodiments of this and other aspects of the invention, the pancreatic cells are islet of Langerhans or fragments thereof. In some embodiments, the pancreatic cells are from a mammal, e.g., a mouse, a rat or a human. In some embodiments, the pancreatic cells are from a subject, where the subject is selected for based on subject's need of additional β-cells. In some embodiments, the pancreatic cells are primary pancreatic cells, e.g. a primary islet cell. In some embodiments, pancreatic cells are not transformed pancreatic cells.

In some embodiments of this and other aspect of the invention, pancreatic cells are isolated from a subject and cultured overnight.

In some embodiments of this and other aspect of the invention, pancreatic cells are trypsinized into cellular clusters of 1-10 cells. Preferably, pancreatic cells are trypsinined into cellular clusters of 1-7 cells, more preferably 1-5, and most preferably 1-3 cells. Trypsinized pancreatic cells can be resuspended in appropriate islet media before plating. In some embodiments, trypsinized pancreatic cells are allowed to recover overnight before plating.

In one embodiment, the method of screening for a candidate compound for stimulating or increasing beta-cell replication comprises:
(a) trypsinizing islets into cellular clusters of 1-3 cells;
(b) allowing the cells to recover overnight;
(c) plating the cells from step (b) into the wells of a 96-well plate, wherein the wells are coated with 804G conditioned media and cellular plating density is 60 k cells/well;
(d) allowing the cells to adhere to surface of the wells for 48 hours;
(e) contacting 1 μM of test compound with the beta-cells for 24 hours;
(f) staining the cells with PDX-1 antibody and Ki-67 and/or PH3 antibody; and assessing beta-cell replication.

As used herein, the term “transformed cells” is art recognized and refers to cells which have converted to a state of unrestrained growth, i.e., they have acquired the ability to grow through an indefinite number of divisions in culture. Transformed cells may be characterized by such terms as neoplastic, anaplastic and/or hyperplastic, with respect to their loss of growth control. In general, term “transformed β-cell” refers to β-cells which exhibit increased capacity to persist in serial subcultures or increased growth rate in vitro.

In some embodiments, the screening method is a high-throughput screening. High-throughput screening (HTS) is a method for scientific experimentation that uses robotics, data processing and control software, liquid handling devices, and sensitive detectors. High-Throughput Screening or HTS allows a researcher to quickly conduct millions of biochemical, genetic or pharmacological tests. High-Throughput Screening are well known to one skilled in the art, for example, those described in U.S. Pat. Nos. 5,976,813; 6,472,144; 6,692,856; 6,824,982; and 7,091,048, and contents of each of which is herein incorporated by reference in its entirety.

HTS uses automation to run a screen of an assay against a library of candidate compounds. An assay is a test for specific activity: usually inhibition or stimulation of a biochemical or biological mechanism. Typical HTS screening libraries or “decks” can contain from 100,000 to more than 2,000,000 compounds.

The key labware or testing vessel of HTS is the microtiter plate: a small container, usually disposable and made of plastic, that features a grid of small, open divots called wells. Modern microplates for HTS generally have either 384, 1536, or 3456 wells. These are all multiples of 96, reflecting the original 96 well microplate with 8×12 9 mm spaced wells.

To prepare for an assay, the researcher fills each well of the plate with the appropriate reagents that he or she wishes to conduct the experiment with, such as a pancreatic cell population. After some incubation time has passed to allow the reagent to absorb, bind to, or otherwise react (or fail to react) with the compounds in the wells, measurements are taken across all the plate's wells, either manually or by a machine. Manual measurements are often necessary when the researcher is using microscopy to (for example) seek changes that a computer could not easily determine by itself. Otherwise, a specialized automated analysis machine can run a number of experiments on the wells such as colorimetric measurements, radioactivity counting, etc. In this case, the machine outputs the result of each experiment as a grid of numeric values, with each number mapping to the value obtained from a single well. A high-capacity analysis machine can measure dozens of plates in the space of a few minutes like this, generating thousands of experimental data points very quickly.

In another aspect, the invention provides a compound selected by the screening assay described herein. It is to be understood that analogs, derivatives, and isomers of the compounds selected by the screening assays described herein are also claimed herein.

### Diagnosis of Diabetes

Type 1 diabetes is an autoimmune disease that results in destruction of insulin-producing beta cells of the pancreas. Lack of insulin causes an increase of fasting blood glucose (around 70-120 mg/dL in nondiabetic people) that begins to appear in the urine above the renal threshold (about 190-200 mg/dl in most people). The World Health Organization defines the diagnostic value of fasting plasma glucose concentration to 7.0 mmol/l (126 mg/dl) and above for Diabetes Mellitus (whole blood 6.1 mmol/l or 110 mg/dl), or 2-hour glucose level of 11.1 mmol/L or higher (200 mg/dL or higher).

Type 1 diabetes can be diagnosed using a variety of diagnostic tests that include, but are not limited to, the following: (1) glycated hemoglobin (A1C) test, (2) random blood glucose test and/or (3) fasting blood glucose test.

The Glycated hemoglobin (A1C) test is a blood test that reflects the average blood glucose level of a subject over the preceding two to three months. The test measures the percentage of blood glucose attached to hemoglobin, which correlates with blood glucose levels (e.g., the higher the blood glucose levels, the more hemoglobin is glycosylated). An A1C level of 6.5 percent or higher on two separate tests is indicative of diabetes. A result between 6 and 6.5 percent is considered prediabetic, which indicates a high risk of developing diabetes.

The Random Blood Glucose Test comprises obtaining a blood sample at a random time point from a subject suspected of having diabetes. Blood glucose values can be expressed in milligrams per deciliter (mg/dL) or millimoles per liter (mmol/L). A random blood glucose level of 200 mg/dL (11.1 mmol/L) or higher indicates the subject likely has diabetes, especially when coupled with any of the signs and symptoms of diabetes, such as frequent urination and extreme thirst.

For the fasting blood glucose test, a blood sample is obtained after an overnight fast. A fasting blood glucose level less than 100 mg/dL (5.6 mmol/L) is considered normal. A fasting blood glucose level from 100 to 125 mg/dL (5.6 to 6.9 mmol/L) is considered prediabetic, while a level of 126 mg/dL (7 mmol/L) or higher on two separate tests is indicative of diabetes.

Type 1 diabetes can also be distinguished from type 2 diabetes using a C-peptide assay, which is a measure of endogenous insulin production. The presence of anti-islet antibodies (to Glutamic Acid Decarboxylase, Insulinoma Associated Peptide-2 or insulin), or lack of insulin resistance, determined by a glucose tolerance test, is also indicative of type 1, as many type 2 diabetics continue to produce insulin internally, and all have some degree of insulin resistance.

Testing for GAD 65 antibodies has been proposed as an improved test for differentiating between type 1 and type 2 diabetes as it appears that the immune system is involved in Type 1 diabetes etiology.

### DEFINITIONS

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

The terms “diabetes” and “diabetes mellitus” are used interchangeably herein. The World Health Organization defines the diagnostic value of fasting plasma glucose concentration to 7.0 mmol/l (126 mg/dl) and above for Diabetes Mellitus (whole blood 6.1 mmol/l or 110 mg/dl), or 2-hour glucose level 11.1 mmol/L or higher (200 mg/dL or higher). Other values suggestive of or indicating high risk for Diabetes Mellitus include elevated arterial pressure 140/90 mm Hg or higher; elevated plasma triglycerides (1.7 mmol/L; 150 mg/dL) and/or low HDL-cholesterol (less than 0.9 mmol/L, 35 mg/dl for men; less than 1.0 mmol/L, 39 mg/dL women); central obesity (males: waist to hip ratio higher than 0.90; females: waist to hip ratio higher than 0.85) and/or body mass index exceeding 30 kg/m²; microalbuminuria, where the urinary albumin excretion rate 20 μg/min or higher, or albumin:creatinine ratio 30 mg/g or higher).

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Other than in the operating examples, or where otherwise indicated, all numbers expressing quantities of ingredients or reaction conditions used herein should be understood as modified in all instances by the term “about.” The term “about” when used in connection with percentages may mean±1%.

The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description.

Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

The terms “decrease”, “reduced”, “reduction”, “decrease” or “inhibit” are all used herein generally to mean a decrease by a statistically significant amount. However, for avoidance of doubt, ““reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means a two standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

As used herein, the term “IC50” refers to the concentration of an inhibitor that produces 50% of the maximal inhibition of ADK activity measurable using the same assay in the absence of the inhibitor. The IC50 can be as measured in vitro or in vivo. The IC50 can be determined by measuring in vitro adenosine kinase activity using a conventional in vitro kinase assay. Inhibition of adenosine kinase can be performed, by example, according to standard procedures well known in the art (Yamada, et al., Comp. Biochem. Physiol. 1982, 71B: 367-372). Typically, adenosine kinase is contacted with the inhibitor compound, typically by adding the compound to an aqueous solution containing the enzyme, radiolabeled substrate adenosine, magnesium chloride and ATP. The enzyme can exist in intact cells or in isolated subcellular fractions containing the enzyme. The enzyme is then maintained in the presence of the inhibitor for a period of time and under suitable physiological conditions. Means for determining maintenance times are well known in the art and depend inter alia on the concentrations of enzyme and the physiological conditions. Suitable physiological conditions are those necessary to maintain adenosine kinase viability and include temperature, acidity, tonicity and the like. By way of example, cells containing adenosine kinase, such as IMR-32 human neuroblastoma cells, are cultured in the presence and absence of an inhibitor. Inhibition is measured as the ability to inhibit phosphorylation of endogenous or externally applied 14C-adenosine by these cells. In some embodiments, ADK activity is measured by an assay as described in Cowart M., et al. Structure-activity studies of 5-substituted pyridopyrimidines as adenosine kinase inhibitors. Bioorg Med Chem Lett 2001, 1:83-86; Davies L P, et al. Halogenated pyrrolopyrimidine analogues of adenosine from marine organisms: Pharmacological activities and potent inhibition of adenosine kinase. Biochem Pharmacol 1984, 33:347-355; Erion M D, et al. Design, synthesis and anticonvulsant activity of the potent adenosine kinase inhibitor GP3269. Nucleosides Nucleotides 1997, 16:1013-1021; Hajduk P J, et al. Design of adenosine kinase inhibitors from the NMR-based screening of fragments. J Med Chem 2000, 3:4781-4786; Jarvis, M F, et al. ABT-702, a novel orally effective adenosine kinase (AK) inhibitor analgesic with anti-inflammatory properties: I. In vitro characterization and acute antinociceptive effects in mice. Pharmacol Exp Ther 2000, 295:1156-1164; Kowaluk E A, Bhagwat S S, Jarvis M F. Adenosine kinase inhibitors. Curr Pharmaceut Des 1998, 4:403-416; Kowaluk E A, et al. Characterization of the effects of adenosine kinase inhibitors on acute thermal nociception in mice. Pharmacol Biochem Behav 1999, 63:83-91; Miller L P, et al. Pre- and peristroke treatment with the adenosine kinase inhibitor, 5′ deoxyiodotubercidin, significantly reduces infarct volume after temporary occlusion of the middle cerebral artery in rats. Neurosci Lett 1996, 220:73-76; Miller R L, Adamczyk D L, Miller W H, et al. Adenosine kinase from rabbit liver. II. Substrate and inhibitor specificity. J Biol Chem 1979, 254:2346-2352; Ugarkar B G, et al. Adenosine kinase inhibitors. 2. Synthesis, enzyme inhibition, and antiseizure activity of diaryltubercidin analogues, J Med Chem 2000, 43:2984-2905; Ugarkar B G, et al. Adenosine kinase inhibitors. 1. Synthesis, enzyme inhibition, and antiseizure activity of 5-iodotubercidin analogues. J Med Chem 2000, 43:2883-2893; Wiesner J B, et al. Adenosine kinase inhibitors as a novel approach to anticonvulsant therapy. J Pharmacol Exp Ther 1999, 289:1669-1677, contents of which are herein incorporated by reference in their entirety.

As used herein, the term “EC50,” refers to the concentration of an activator that produces 50% of maximal activation of AMPK activity measurable using the same assay in the absence of the activator. Stated differently, the “EC50” is the concentration of activator that gives 50% activation, when 100% activation is set at the amount of activity that does not increase with the addition of more activator. The EC50 can be as measured in vitro or in vivo. The EC50 can be determined by measuring in vitro AMPK activity using a conventional in vitro kinase assay. In some embodiments, AMPK activity is by an assay as described in Gorton, J M, et al., 5-Aminoimidazole-4-carboxamide ribonucleoside: a specific method for activating AMP-activated protein kinase in intact cells? Eur. J. Biochem. 1995, 229:558-565, contents of which are herein incorporated by reference in their entirety.

“Impaired glucose tolerance” (IGT) is defined as having a blood glucose level that is higher than normal, but not high enough to be classified as Diabetes Mellitus. A subject with IGT will have two-hour glucose levels of 140 to 199 mg/dL (7.8 to 11.0 mmol) on the 75 g oral glucose tolerance test. These glucose levels are above normal but below the level that is diagnostic for Diabetes. Subjects with impaired glucose tolerance or impaired fasting glucose have a significant risk of developing Diabetes and thus are an important target group for primary prevention.

“Normal glucose levels” is used interchangeably with the term “normoglycemic” and refers to a fasting venous plasma glucose concentration of less than 6.1 mmol/L (110 mg/dL). Although this amount is arbitrary, such values have been observed in subjects with proven normal glucose tolerance, although some may have IGT as measured by oral glucose tolerance test (OGTT). A baseline value, index value, or reference value in the context of the present invention and defined herein can comprise, for example, “normal glucose levels.”

A “pre-diabetic condition” refers to a metabolic state that is intermediate between normal glucose homeostasis, metabolism, and states seen in frank Diabetes Mellitus. Pre-diabetic conditions include, without limitation, Metabolic Syndrome (“Syndrome X”), Impaired Glucose Tolerance (IGT), and Impaired Fasting Glycemia (IFG). IGT refers to post-prandial abnormalities of glucose regulation, while IFG refers to abnormalities that are measured in a fasting state. The World Health Organization defines values for IFG as a fasting plasma glucose concentration of 6.1 mmol/L (100 mg/dL) or greater (whole blood 5.6 mmol/L; 100 mg/dL), but less than 7.0 mmol/L (126 mg/dL) (whole blood 6.1 mmol/L; 110 mg/dL). Metabolic Syndrome according to National Cholesterol Education Program (NCEP) criteria are defined as having at least three of the following: blood pressure 130/85 mm Hg or higher; fasting plasma glucose 6.1 mmol/L or higher; waist circumference >102 cm (men) or >88 cm (women); triglycerides 1.7 mmol/L or higher; and HDL cholesterol <1.0 mmol/L (men) or 1.3 mmol/L (women).

“Complications related to type 2 Diabetes” or “complications related to a pre-diabetic condition” can include, without limitation, diabetic retinopathy, diabetic nephropathy, blindness, memory loss, renal failure, cardiovascular disease (including coronary artery disease, peripheral artery disease, cerebrovascular disease, atherosclerosis, and hypertension), neuropathy, autonomic dysfunction, hyperglycemic hyperosmolar coma, or combinations thereof.

As used herein, the term “HBA1c” refers to glycosylated hemoglobin or glycosylated hemoglobin, and is an indicator of blood glucose levels over a period of time (e.g., 2-3 months). The level of HBA1c is “reduced” if there is a decrease of at least 10%, at least 20%, at least 30%, at least 40%, at least 50%, at least 60%, at least 70%, at least 80%, at least 90%, at least 95%, or more upon treatment with a compound described herein compared to the level of HBA1c prior to the onset of treatment in the subject. Similarly, ketone bodies are “reduced” if there is a decrease of at least 20%, at least 30%, at least 40%, at least 50%, at least 60%, at least 70%, at least 80%, at least 90%, at least 95%, or more upon treatment with a compound described herein.

For simplicity, chemical moieties are defined and referred to throughout can be univalent chemical moieties (e.g., alkyl, aryl, etc.) or multivalent moieties under the appropriate structural circumstances clear to those skilled in the art. For example, an “alkyl” moiety can be referred to a monovalent radical (e.g. CH₃—CH₂—), or in other instances, a bivalent linking moiety can be “alkyl,” in which case those skilled in the art will understand the alkyl to be a divalent radical (e.g., —CH₂—CH₂—), which is equivalent to the term “alkylene.” Similarly, in circumstances in which divalent moieties are required and are stated as being “alkoxy”, “alkylamino”, “aryloxy”, “alkylthio”, “aryl”, “heteroaryl”, “heterocyclic”, “alkyl” “alkenyl”, “alkynyl”, “aliphatic”, or “cycloalkyl”, those skilled in the art will understand that the terms “alkoxy”, “alkylamino”, “aryloxy”, “alkylthio”, “aryl”, “heteroaryl”, “heterocyclic”, “alkyl”, “alkenyl”, “alkynyl”, “aliphatic”, or “cycloalkyl” refer to the corresponding divalent moiety.

The term “halo” refers to any radical of fluorine, chlorine, bromine or iodine.

The term “acyl” refers to an alkylcarbonyl, cycloalkylcarbonyl, arylcarbonyl, heterocyclylcarbonyl, or heteroarylcarbonyl substituent, any of which may be further substituted by substituents. Exemplary acyl groups include, but are not limited to, (C₁-C₆)alkanoyl (e.g., formyl, acetyl, propionyl, butyryl, valeryl, caproyl, t-butylacetyl, etc.), (C₃-C₆)cycloalkylcarbonyl (e.g., cyclopropylcarbonyl, cyclobutylcarbonyl, cyclopentylcarbonyl, cyclohexylcarbonyl, etc.), heterocyclic carbonyl (e.g., pyrrolidinylcarbonyl, pyrrolid-2-one-5-carbonyl, piperidinylcarbonyl, piperazinylcarbonyl, tetrahydrofuranylcarbonyl, etc.), aroyl (e.g., benzoyl) and heteroaroyl (e.g., thiophenyl-2-carbonyl, thiophenyl-3-carbonyl, furanyl-2-carbonyl, furanyl-3-carbonyl, 1H-pyrroyl-2-carbonyl, 1H-pyrroyl-3-carbonyl, benzo[b]thiophenyl-2-carbonyl, etc.). In addition, the alkyl, cycloalkyl, heterocycle, aryl and heteroaryl portion of the acyl group may be any one of the groups described in the respective definitions.

The term “alkyl” refers to saturated non-aromatic hydrocarbon chains that may be a straight chain or branched chain, containing the indicated number of carbon atoms (these include without limitation methyl, ethyl, propyl, allyl, or propargyl), which may be optionally inserted with N, O, S, SS, SO₂, C(O), C(O)O, OC(O), C(O)N or NC(O). For example, C₁-C₆ indicates that the group may have from 1 to 6 (inclusive) carbon atoms in it.

The term “alkenyl” refers to an alkyl that comprises at least one double bond. Exemplary alkenyl groups include, but are not limited to, for example, ethenyl, propenyl, butenyl, 1-methyl-2-buten-1-yl and the like.

The term “alkynyl” refers to an alkyl that comprises at least one triple bond.

The term “alkoxy” refers to an —O-alkyl radical.

The term “aminoalkyl” refers to an alkyl substituted with an amino.

The term “mercapto” refers to an —SH radical.

The term “thioalkoxy” refers to an —S-alkyl radical.

The term “aryl” refers to monocyclic, bicyclic, or tricyclic aromatic ring system wherein 0, 1, 2, 3, or 4 atoms of each ring may be substituted by a substituent. Exemplary aryl groups include, but are not limited to, phenyl, naphthyl, anthracenyl, azulenyl, fluorenyl, indanyl, indenyl, naphthyl, phenyl, tetrahydronaphthyl, and the like.

The term “arylalkyl” refers to an alkyl substituted with an aryl.

The term “cyclyl”, “cyclic” or “cycloalkyl” refers to saturated and partially unsaturated cyclic hydrocarbon groups having 3 to 12 carbons, for example, 3 to 8 carbons, and, for example, 3 to 6 carbons, wherein the cycloalkyl group additionally may be optionally substituted. Exemplary cycloalkyl groups include, but are not limited to, cyclopropyl, cyclobutyl, cyclopentyl, cyclopentenyl, cyclohexyl, cyclohexenyl, cycloheptyl, cyclooctyl, and the like.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2, 3, or 4 atoms of each ring may be substituted by a substituent. Exemplary heteroaryl groups include, but are not limited to, pyridyl, furyl or furanyl, imidazolyl, benzimidazolyl, pyrimidinyl, thiophenyl or thienyl, pyridazinyl, pyrazinyl, quinolinyl, indolyl, thiazolyl, naphthyridinyl, and the like.

The term “heteroarylalkyl” refers to an alkyl substituted with a heteroaryl.

The term “heterocyclyl”, “heterocycle” or “heterocyclic” refers to a nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Exemplary heterocyclyl groups include, but are not limited to piperazinyl, pyrrolidinyl, dioxanyl, morpholinyl, tetrahydrofuranyl, and the like.

The term “haloalkyl” refers to an alkyl group having one, two, three or more halogen atoms attached thereto. Exemplary haloalkyl groups include, but are not limited to chloromethyl, bromoethyl, trifluoromethyl, and the like.

The term “optionally substituted” means that the specified group or moiety, such as an alkyl, aryl group, heteroaryl group and the like, is unsubstituted or is substituted with one or more (typically 1-4 substituents) independently selected from the group of substituents listed below in the definition for “substituents” or otherwise specified.

The term “substituents” refers to a group “substituted” on an alkyl, alkenyl, alkynyl, cycloalkyl, aryl, heterocyclyl, heteroaryl, acyl, amino group at any atom of that group. Suitable substituents include, without limitation, halo, hydroxy, oxo, nitro, haloalkyl, alkyl, alkenyl, alkynyl, alkaryl, aryl, aralkyl, alkoxy, aryloxy, amino, acylamino, alkylcarbamoyl, arylcarbamoyl, aminoalkyl, alkoxycarbonyl, carboxy, hydroxyalkyl, alkylthio, CF₃, N-morpholino, phenylthio, alkanesulfonyl, arenesulfonyl, alkanesulfonamido, arenesulfonamido, aralkylsulfonamido, alkylcarbonyl, acyloxy, cyano or ureido. In some embodiments, substituent can itself be optionally substituted. In some cases, two substituents, together with the carbons to which they are attached to can form a ring.

The compounds described herein and their salts include asymmetric carbon atoms and may therefore exist as single stereoisomers, racemates, and as mixtures of enantiomers and diastereomers. Typically, such compounds will be prepared as a racemic mixture. If desired, however, such compounds can be prepared or isolated as pure stereoisomers, i.e., as individual enantiomers or diastereomers, or as stereoisomer-enriched mixtures. As discussed in more detail below, individual stereoisomers of compounds are prepared by synthesis from optically active starting materials containing the desired chiral centers or by preparation of mixtures of enantiomeric products followed by separation or resolution, such as conversion to a mixture of diastereomers followed by separation or recrystallization, chromatographic techniques, use of chiral resolving agents, or direct separation of the enantiomers on chiral chromatographic columns. Starting compounds of particular stereochemistry are either commercially available or are made by the methods described below and resolved by techniques well-known in the art.

As used herein, the terms “stereoisomer” or “optical isomer” mean a stable isomer that has at least one chiral atom or restricted rotation giving rise to perpendicular dissymmetric planes (e.g., certain biphenyls, allenes, and spiro compounds) and can rotate plane-polarized light. Because asymmetric centers and other chemical structure exist in the compounds described herein as suitable for use in the present invention which may give rise to stereoisomerism, the invention contemplates stereoisomers and mixtures thereof. The term “enantiomers” means a pair of stereoisomers that are non-superimposable mirror images of each other. The term “diastereoisomers” or “diastereomers” mean optical isomers which are not mirror images of each other. The term “racemic mixture” or “racemate” mean a mixture containing equal parts of individual enantiomers. The term “non-racemic mixture” means a mixture containing unequal parts of individual enantiomers.

The term “enantiomeric enrichment” as used herein refers to the increase in the amount of one enantiomer as compared to the other. A convenient method of expressing the enantiomeric enrichment achieved is the concept of enantiomeric excess, or “ee”, which is found using the following equation:

ee=100×(E¹−E²)/(E¹+E²),

wherein E¹ is the amount of the first enantiomer and E² is the amount of the second enantiomer.

In some embodiments, compound described herein have an enantiomeric excess of at least 1%, 5%, 10%, 20%, 30%, 40%, 50%, 60%, 70%, 80%, 90%, 95% or more. Generally, an ee of greater than 90% is preferred, an ee of greater than 95% is most preferred and an ee of greater than 99% is most especially preferred.

Enantiomeric enrichment is readily determined by one of ordinary skill in the art using standard techniques and procedures, such as gas or high performance liquid chromatography with a chiral column. Choice of the appropriate chiral column, eluent and conditions necessary to effect separation of the enantiomeric pair is well within the knowledge of one of ordinary skill in the art. In addition, the enantiomers of compounds can be resolved by one of ordinary skill in the art using standard techniques well known in the art, such as those described by J. Jacques, et al., “Enantiomers, Racemates, and Resolutions”, John Wiley and Sons, Inc., 1981. Examples of resolutions include recrystallization techniques or chiral chromatography.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The present invention may be defined in any of the following numbered paragraphs:
1. A method of increasing β-cell replication in a population of pancreatic cells, the method comprising: contacting a population of pancreatic cells with an inhibitor of adenosine kinase (ADK), an inhibitor of S-Adenosylhomocysteine hydrolase (SAHH), or an activator of AMP activated protein kinase (AMPK).
2. The method of paragraph 1, wherein the inhibitor of adenosine kinase is of formula (I):

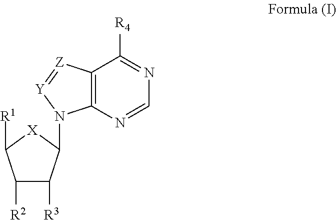

wherein:
R¹ is H, optionally substituted C₁-C₆ alkyl, optionally substituted C₂-C₆ alkenyl, optionally substituted C₂-C₆ alkynyl, OR⁵, SR⁵, N(R⁶)₂, (CH₂)ₘR⁷, or R¹ and R² together with the atoms they are attached to form 5-8 membered heterocycle which can be optionally substituted;
R² and R³ are each independently H, OR⁵, SR⁵, N(R⁵)₂, or R² and R³ together with the atoms they are attached to form 5-8 membered heterocyclyl which can be optionally substituted;
R⁴ is H, halogen, CN, N₂, OR⁵, SR⁵, N(R⁵)₂, optionally substituted C₁-C₆ alkyl, optionally substituted C₂-C₆ alkenyl, optionally substituted C₂-C₆ alkynyl, optionally substituted aryl, optionally substituted heteroaryl, optionally substituted cyclyl, optionally substituted heterocyclyl, optionally substituted arylalkyl, or optionally substituted heteroarylalkyl;
R⁵ is independently for each occurrence H, C(O)R⁷, C(O)OR⁷, C(O)N(R⁷)₂, optionally substituted C₁-C₆ alkyl, optionally substituted C₂-C₆ alkenyl, optionally substituted C₂-C₆ alkynyl, optionally substituted aryl, optionally substituted heteroaryl, optionally substituted cyclyl, optionally substituted heterocyclyl, optionally substituted arylalkyl, optionally substituted heteroarylalkyl, or the two R⁵ taken together with the nitrogen atom to which they are attached form a 5-to-7 membered ring optionally comprising 1-3-additional heteroatoms selected from N, O or S;
R⁶ is R⁵, OR⁵, SR⁵, N(R⁵)₂, N₂, CN, halogen, or

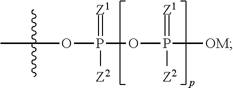

R⁷ is independently for each occurrence H, optionally substituted C₁-C₆ alkyl, optionally substituted C₂-C₆ alkenyl, optionally substituted C₂-C₆ alkynyl, optionally substituted aryl, optionally substituted heteroaryl, optionally substituted cyclyl, optionally substituted heterocyclyl, optionally substituted arylalkyl, or optionally substituted heteroarylalkyl;
X is O, S, NH, or CH₂;
Y and Z are each independently N or CR⁸;
R⁸ is independently for each occurrence H, halogen, CN, C(O)R⁷, C(O)OR⁷, C(O)N(R⁷)₂, optionally substituted C₁-C₆ alkyl, optionally substituted C₂-C₆ alkenyl, optionally substituted C₂-C₆ alkynyl, optionally substituted aryl, optionally substituted heteroaryl, optionally substituted cyclyl, optionally substituted heterocyclyl, optionally substituted arylalkyl, or optionally substituted heteroarylalkyl;
Z¹ is independently for each occurrence O or S;
Z² is independently for each occurrence OM, SM, OR⁵, SR⁵, N(R⁵)₂, optionally substituted C₁-C₆ alkyl, optionally substituted C₂-C₆ alkenyl, optionally substituted C₂-C₆ alkynyl, optionally substituted aryl, optionally substituted heteroaryl, optionally substituted cyclyl, optionally substituted heterocyclyl, optionally substituted aryl alkyl, or optionally substituted heteroaryl alkyl;
M is an alkali metal cation;
m is 1, 2, 3, or 4;
n is 0, 1, or 2; and
pharmaceutically acceptable salts and amides thereof.
3. The method of paragraph 1, wherein the inhibitor of adenosine kinase is of formula (II):

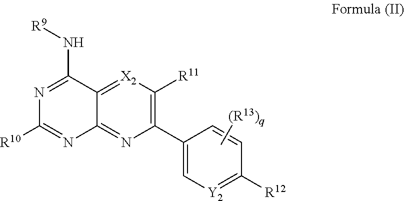

wherein:
each R⁹ is independently H, optionally substituted C₁-C₆ alkyl, optionally substituted C₂-C₆ alkenyl, optionally substituted C₂-C₆ alkynyl, optionally substituted aryl, optionally substituted heteroaryl, optionally substituted cyclyl, optionally substituted heterocyclyl, optionally substituted arylalkyl, optionally substituted heteroarylalkyl, or the two R⁹ taken together with the nitrogen atom to which they are attached form a 5-to-7 membered ring which optionally comprises 1-3-additional heteroatoms selected from N, O or S;
R¹⁰, R¹¹ and R¹² are each independently H, OR¹⁴, N(R¹⁴)₂, N₂, NO₂, CN, halogen, optionally substituted C₁-C₆ alkyl, optionally substituted C₂-C₆ alkenyl, optionally substituted C₂-C₆ alkynyl, optionally substituted aryl, optionally substituted heteroaryl, optionally substituted cyclyl, optionally substituted heterocyclyl, optionally substituted arylalkyl, or optionally substituted heteroarylalkyl;
R¹³ is independently for each occurrence halogen, CN, NH₂, or optionally substituted C₁-C₆ alkyl;
R¹⁴ is H, optionally substituted C₁-C₆ alkyl, optionally substituted C₂-C₆ alkenyl, optionally substituted C₂-C₆ alkynyl, optionally substituted aryl, optionally substituted heteroaryl, optionally substituted cyclyl, optionally substituted heterocyclyl, optionally substituted arylalkyl, optionally substituted heteroarylalkyl, or the two R¹⁴ taken together with the nitrogen atom to which they are attached form a 5-to-7 membered ring which optionally comprises 1-3-additional heteroatoms selected from N, O or S;
X₂ is N or CR¹⁵;
R¹⁵ is NHR¹⁶, optionally substituted C₁-C₆ alkyl, optionally substituted C₂-C₆ alkenyl, optionally substituted C₂-C₆ alkynyl, optionally substituted aryl, optionally substituted heteroaryl, optionally substituted cyclyl, optionally substituted heterocyclyl, optionally substituted arylalkyl, or optionally substituted heteroarylalkyl;
R¹⁶ is H, optionally substituted C₁-C₆ alkyl, optionally substituted C₂-C₆ alkenyl, optionally substituted C₂-C₆ alkynyl, optionally substituted aryl, optionally substituted heteroaryl, optionally substituted cyclyl, optionally substituted heterocyclyl, optionally substituted arylalkyl, or optionally substituted heteroarylalkyl;
Y₂ is N or CH;
q is 0, 1, 2, or 3; and
pharmaceutically acceptable salts and amides thereof.
4. The method of any of paragraphs 1-3, wherein IC50 of the ADK inhibitor is less than or equal to 500 nM, 250 nM, 200 nM, 100 nM, 75 nM, 50 nM, 25 nM, 10 nM, 1 nM, 0.1 nM, 0.01 nM, 0.001 nM.
5. The method of any of paragraphs 1-4, wherein the ADK activity is inhibited or lowered by at least 5%, 10%, 20%, 30%, 40%, 50%, 50%, 70%, 80%, 90%, 95%, 98%, or 100% (e.g. complete loss of activity) relative to an uninhibited control.
6. The method of any of paragraphs 1-5, wherein the ADK inhibitor is selected from the group consisting of aristeromycin, 5′-deoxyadenosine, 5′-aminoadenosine, 5′-deoxy-5-iodotubercidin, 5-iodotubercidin (A10), 7-deaza-7-iodo-2′,3′-dideoxyadenosine, nor-aristeromycin, nor-tubercidin, A-134974, Toyocamycin, GP-515 ((2R,3R,4S,5R)-2-(4-amino-3-bromo-1H-pyrazolo[3,4-d]pyrimidin-1-yl)-5-(aminomethyl)-tetrahydrofuran-3,4-diol), GP-3269 ((2R,3R,4S,5R)-2-(4-(4-fluorophenylamino)-5-phenyl-7H-pyrrolo[2,3-d]pyrimidin-7-yl)-tetrahydro-5-methylfuran-3,4-diol), GP-683 ((2R,3S,4R,5R)-tetrahydro-2-methyl-5-(5-phenyl-4-(phenylamino)-7H-pyrrolo[2,3-d]pyrimidin-7-yl)furan-3,4-diol), GP-947 ((2S,3S,4R,5R)-tetrahydro-2-methyl-5-(5-phenyl-4-(phenylamino)-7H-pyrrolo[2,3-d]pyrimidin-7-yl)furan-3,4-diol), ABT-702 (5-(3-bromophenyl)-7-(6-morpholinopyridin-3-yl)pyrido[2,3-d]pyrimidin-4-amine, B8), compound 1 ((2R,3R,4S,5R)-2-(4-amino-5-iodo-7H-pyrrolo[2,3-d]pyrimidin-7-yl)-5-(aminomethyl)-tetrahydrofuran-3,4-diol), compound 2 ((2R,3R,4S,5R)-2-(4-chloro-5-iodo-7H-pyrrolo[2,3-d]pyrimidin-7-yl)-5-(aminomethyl)-tetrahydrofuran-3,4-diol), compound 3 ((1S,2R,3S,5R)-3-amino-5-(6-amino-9H-purin-9-yl)cyclopentane-1,2-diol), compound 4 ((1S,2R,3S,5R)-3-amino-5-(7-amino-3H-[1,2,3]triazolo[4,5-d]pyrimidin-3-yl)cyclopentane-1,2-diol), compound 5 ((1S,2R,3S,4R)-4-(4-amino-5-iodo-7H-pyrrolo[2,3-d]pyrimidin-7-yl)cyclopentane-1,2,3-triol), compound 6 (7-(4-(dimethylamino)phenyl)pteridin-4-amine), compound 7 (5-(3-bromophenyl)-7-(4-(dimethylamino)phenyl)pyrido[2,3-d]pyrimidin-4-amine), compound 8 (5-(2-bromobenzyl)-7-(6-morpholinopyridin-3-yl)pyrido[2,3-d]pyrimidin-4-amine), compound 9 (5-cyclohexyl-7-(6-morpholinopyridin-3-yl)pyrido[2,3-d]pyrimidin-4-amine), compound 10 (5-(tetrahydro-2H-pyran-4-yl)-7-(6-morpholinopyridin-3-yl)pyrido[2,3-d]pyrimidin-4-amine), compound 11 (5-(1-(2-bromophenyl)ethyl)-7-(6-morpholinopyridin-3-yl)pyrido[2,3-d]pyrimidin-4-amine), compound 12 (5-(2-methylpent-4-en-2-yl)-7-(6-morpholinopyridin-3-yl)pyrido[2,3-d]pyrimidin-4-amine), compound 13 (N5-((1H-indol-3-yl)methyl)-7-(6-morpholinopyridin-3-yl)pyrido[2,3-d]pyrimidine-4,5-diamine), and combinations thereof.
7. The method of paragraph 1, wherein the activator of AMPK is of formula (III):

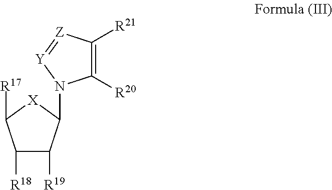

wherein:
R¹⁷ is H, optionally substituted C₁-C₆ alkyl, optionally substituted C₂-C₆ alkenyl, optionally substituted C₂-C₆ alkynyl, OR²², SR²², N(R²²)₂, (CH₂)ₘR²³, or R¹⁷ and R¹⁸ together with the atoms they are attached to form 5-8 membered heterocycle which can be optionally substituted;
R¹⁸ and R¹⁹ are each independently H, OR²², SR²², N(R²²)₂, O or R¹⁸ and R¹⁹ together with the atoms they are attached to form 5-8 membered heterocycle which can be optionally substituted;
R²⁰ and R²¹ are each independently halogen, CN, N₂, OR²², SR²², N(R²²)₂, C(O)R²⁴, C(O)OR²⁴, C(O)N(R²⁴)₂, optionally substituted C₁-C₆ alkyl, optionally substituted C₂-C₆ alkenyl, optionally substituted C₂-C₆ alkynyl, optionally substituted aryl, optionally substituted heteroaryl, optionally substituted cyclyl, optionally substituted heterocyclyl, optionally substituted arylalkyl, or optionally substituted heteroarylalkyl;
R²² is independently for each occurrence H, C(O)R²⁴, C(O)OR²⁴, C(O)N(R²⁴)₂, optionally substituted C₁-C₆ alkyl, optionally substituted C₂-C₆ alkenyl, optionally substituted C₂-C₆ alkynyl, optionally substituted aryl, optionally substituted heteroaryl, optionally substituted cyclyl, optionally substituted heterocyclyl, optionally substituted arylalkyl, or optionally substituted heteroarylalkyl;
R²³ is R²², OR²², SR²², N(R²²)₂, N₂, CN, halogen, or

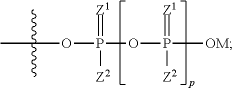

R²⁴ is independently for each occurrence H, optionally substituted C₁-C₆ alkyl, optionally substituted C₂-C₆ alkenyl, optionally substituted C₂-C₆ alkynyl, optionally substituted aryl, optionally substituted heteroaryl, optionally substituted cyclyl, optionally substituted heterocyclyl, optionally substituted arylalkyl, or optionally substituted heteroarylalkyl;
X is O, S, NH, or CH₂;
Y and Z are each independently N or CR²⁵;
R²⁵ is independently for each occurrence H, halogen, CN, C(O)R²⁴, C(O)OR²⁴, C(O)N(R²⁴)₂, optionally substituted C₁-C₆ alkyl, optionally substituted C₂-C₆ alkenyl, optionally substituted C₂-C₆ alkynyl, optionally substituted aryl, optionally substituted heteroaryl, optionally substituted cyclyl, optionally substituted heterocyclyl, optionally substituted arylalkyl, or optionally substituted heteroarylalkyl;
Z¹ is independently for each occurrence O or S;
Z² is independently for each occurrence H, OM, SM, OR²², SR²², N(R²²)₂, optionally substituted C₁-C₆ alkyl, optionally substituted C₂-C₆ alkenyl, optionally substituted C₂-C₆ alkynyl, optionally substituted aryl, optionally substituted heteroaryl, optionally substituted cyclyl, optionally substituted heterocyclyl, optionally substituted arylalkyl, or optionally substituted heteroarylalkyl;
M is an alkali metal cation;
m is 1, 2, 3, or 4;
n is 0, 1, or 2; and
pharmaceutically acceptable salts and amides thereof.
8. The method of paragraph 1 or 7, wherein activity of the AMP-activated kinase is increased by at least 5%, 10%, 20%, 30%, 40%, 50%, 50%, 70%, 80%, 90%, 1-fold, 1.1-fold, 1.5-fold, 2-fold, 3-fold, 4-fold, 5-fold or more relative to an unactivated control.
9. The method of any of paragraphs 1, 7 or 8, wherein EC50 of the AMPK activator is less than or equal to 500 nM, 250 nM, 100 nM, 50 nM, 10 nM, 1 nM, 0.1 nM, 0.01 nM or 0.001 nM.
10. The method of any of paragraphs 1, 7-9, wherein the AMPK activator is 5-aminoimidazole-4-carboxamide ribonucleoside (AICAR).
11. The method of paragraph 1, wherein IC50 of the SAHH inhibitor is less than or equal to 500 nM, 250 nM, 200 nM, 100 nM, 75 nM, 50 nM, 25 nM, 10 nM, 1 nM, 0.1 nM, 0.01 nM, 0.001 nM.
12. The method of paragraph 1 or 11, wherein the SAHH activity is inhibited or lowered by at least 5%, 10%, 20%, 30%, 40%, 50%, 50%, 70%, 80%, 90%, 95%, 98%, or 100% (e.g. complete loss of activity) relative to an uninhibited control.
13. The method of any of paragraphs 1, 11 or 12, wherein the SAHH inhibitor is selected from the group consisting of 9(S)-(2,3-dihydroxypropyl)adenine [(S)-DHPA]; D-eritadine; (R,S)-3-adenine-9-yl-2-hydroxypropanoic acid [(R,S)-AHPA]; adenosine (Ado) dialdehyde; 3-deazaadenosine (c3-Ado); aristeromycin (Ari); neplanocin A (NPA or NpcA); dihydroxycyclopentenyladenine (DHCeA); dihydroxycyclopentenyl-3-deazaadenine (c3-DHCeA); dihydroxycyclopentanyladenine (DHCaA); dihydroxycyclopentanyl-3-deazaadenine (c3-DHCaA); 3-deazaneplanocin A (c3-NpcA); 3-deazaaristeromycin (c3Ari); carbocyclic-3-deazaadenosine (C-c3Ado); 6′-Cmethylneplanocin A; 2′-deoxyadenosine; tubercidin; ribavirin; pyraazofurin; 2′-deoxy-2′-chloroadenosine; isopentenyladenosine; methylthioadenosine (MTA); 9-β-arabinofuranosyladenine (Ara-A, vidarabine); 2′-Deoxyadenosine; N-methylaristeromycin, 8-azaaristeromycin and 3-deazaaristeromycin, and their dialdehyde and diol derivatives; (±)-5Noraristeromycin and its 2,6-diamino-analogue; 2′-deoxy-aristeromycin; 3′-deoxy-aristeromycin; 3′-amino-3′-deoxy-aristeromycin; 3′-amino-3′-deoxyarabinofuranosyl-aristeromycin; 6′-hydroxy-aristeromycin; 6′-mercapto-aristeromycin; 8′-bromo-aristeromycin; 8-hydroxyaristeromycin, aristeromycin-3′-cyclic phosphate, aristeromycin-6′-cyclic phosphate; 2-fluoro-5-adenosylhomocysteine (2-FSAH); S-Adenosyl-L-homocysteine sulfoxide; S-Adenosyl-Lhomocysteine sulfone; S-aristeromycinyl-L-homocysteine; 5′-S-(3-carboxyl-4-nitrophenyl)thioadenosine; 5′-S(methyl)-5′-S-(butyl)thioadenosine; and any combinations thereof.
14. The method of any of paragraphs 1-13, wherein the pancreatic cells are from a subject, and wherein the subject is in need of additional β-cells.
15. The method of any of paragraphs 1-14, wherein the pancreatic cells are from a subject, and wherein the subject is not in need of additional β-cells.
16. The method of any of paragraphs 1-15, wherein the subject is a mammal.
17. The method of any of paragraphs 1-16, wherein subject is a human.
18. The method of any of paragraphs 1-16, wherein the subject is a mouse.
19. The method of any of paragraphs 1-18, wherein the pancreatic cells are primary pancreatic cells.
20. The method of any of paragraphs 1-19, wherein the pancreatic cells are derived from de-differentiated cells.
21. The method of any of paragraphs 1-20, wherein the contact is in vitro.
22. The method of any of paragraphs 1-20, wherein the contact is ex vivo.
23. The method of any of paragraphs 1-20, wherein the contact is in vivo.
24. The method of paragraph 23, wherein in vivo contact is in a mammal.
25. The method of paragraph 24, wherein in vivo contact is in a mouse.
26. The method of paragraph 24, wherein in vivo contact is in a human.
27. The method of paragraph 23, wherein the in vivo contact is in a subject, where the subject is in need of additional β-cells.
28. The method of paragraph 27, wherein the subject suffers from Type 1 diabetes.
29. The method of paragraph 27, wherein the subject suffers from Type 2 diabetes.
30. The method of paragraph 27, wherein the subject is a mammal.
31. The method of any of paragraph 27-30, wherein the subject is a human.
32. The method of any of paragraphs 1-31, wherein β-cell replication increases by at least 5%, 10%, 20%, 30%, 40%, 50%, 50%, 70%, 80%, 90%, 1-fold, 1.1-fold, 1.5-fold, 2-fold, 3-fold, 4-fold, 5-fold or more relative to a control.
33. A high throughput assay for screening compounds that increases β-cell replication in a population of pancreatic cells, the assay comprising:
   (a) contacting a population or preparation of pancreatic cells with a test compound, wherein the pancreatic cells are primary pancreatic cells;
   (b) assessing beta-cell replication or growth; and
   (c) selecting the compound that increases or enhances β-cell replication or growth.
34. The assay of paragraph 33, wherein the pancreatic cells are cultured in a cell culture vessel, surface of which is coated with conditioned media from rat bladder carcinoma cell line 804G.
35. The assay of any of paragraphs 33-34, wherein the step of assessing beta-cell replication comprises detecting a beta-cell marker and a cell-replication marker.
36. The assay of paragraph 35, wherein the cell-replication marker is Ki-67 or PH3.
37. The assay of any of paragraphs 35-36, wherein the β-cell marker is selected from the group consisting of PDX-1, insulin, c-peptide, amylin, E-cadherin, Hnf3β, PCI/3, Beta2, Nkx2.2, Nkx6.1, GLUT2, PC2, ZnT-8, MAFA, MAFB, and combinations thereof.
38. The assay of any of paragraphs 35-37, wherein the β-cell marker is PDX-1.
39. The assay of any of paragraphs 35-38, wherein the β-cell marker is not insulin.
40. The assay of any of paragraphs 33-39, wherein the test compound has a concentration in the range of 0.1 nM to 1000 mM.
41. The assay of any of paragraphs 33-40, wherein the assay is performed at a temperature in the range of about 15° C. to about 55° C.
42. The assay of any of paragraphs 33-41, wherein the test compound is contacted with the pancreatic cells for at least 1 hour.
43. The assay of any of paragraphs 30-42, wherein the test compound increases beta-cell replication or growth by at least 5%, 10%, 20%, 30%, 40%, 50%, 50%, 70%, 80%, 90%, 1-fold, 1.1-fold, 1.5-fold, 2-fold, 3-fold, 4-fold, 5-fold, 10-fold, 50-fold, 100-fold or more higher relative to an untreated control.
44. The assay of any of paragraphs 33-43, wherein the pancreatic cells are not pancreatic cell lines.
45. The assay of any of paragraphs 33-44, wherein the pancreatic cell population is an islet of Langerhan or a fragment thereof.
46. The assay of any of paragraphs 33-45, wherein the pancreatic cells are primary pancreatic β-cells.
47. The assay of any of paragraphs 33-46, wherein the pancreatic cells are allowed to adhere to the surface of the cell culture vessel for about 12 to 48 hours before contact with the test compound.
48. The assay of any of paragraphs 33-47, wherein the pancreatic cells are derived from de-differentiated cells.
49. The assay of any of paragraphs 33-48, wherein the de-differentiated cells are induced pluripotent stem cells.
50. The assay of any of paragraphs 33-49, the assay comprising:
   (a) trypsinizing islets of Langerhans into cellular clusters of 1-3 cells;
   (b) allowing the cells to recover overnight;
   (c) plating the cells from step (b) into the wells of a 96-well plate, wherein the wells are coated with 804G conditioned media and cellular plating density is 60 k cells/well;
   (d) allowing the cells to adhere to surface of the wells for at 48 hours;
   (e) contacting 1 μM of test compound with the beta-cells for 24 hours;
   (f) staining the cells with PDX-1 antibody and Ki-67 and/or PH3 antibody;
   (g) assessing beta-cell replication; and
   (h) selecting the compound that increases or enhances β-cell replication.

To the extent not already indicated, it will be understood by those of ordinary skill in the art that any one of the various embodiments herein described and illustrated may be further modified to incorporate features shown in any of the other embodiments disclosed herein.

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

### EXAMPLES

### Example 1

### Screening Assay

Rat islets were isolated as previously described in Gotoh M, Maki T, Kiyoizumi T, Satomi S, Monaco A P: An improved method for isolation of mouse pancreatic islets. Transplantation 40:437-438, 1985, contents of which are herein incorporated by reference in their entirety. Isolated islets were cultured overnight in a tissue culture incubator. The following morning, islets were trypsinized into cellular clusters of 1-3 cells, re-suspended in islet media (Mediatech 99-786-CV; 10% FBS serum (Valley Biomedial BS3033); 8.3 mM Glucose (Sigma G7528); 1× Penicillin/Streptomycin (Invitrogen 15070-063); 1× Glutamax (Invitrogen 35050-079)) and plated into the wells of a 96-well plate (Sigma CLS3904) that had been coated with 804G (a rat bladder carcinoma cell line) conditioned media. The cellular plating density was 60 k cells/well and greater than 95% viability was confirmed at the time of plating. The islet cells were allowed 48-hours to adhere at which time the media was changed (Mediatech 99-786-CV 2% serum, 5 mM glucose, 1× pen/strep, 1× glutamax) and the cells were treated with the test compounds. For screening, compounds were tested at 1 uM concentration in duplicate. After 24-hours of compound treatment, cells were fixed with fresh 4% PFA (Electron Microscopy Services 15710) for 20 minutes and washed with PBS (VWR 45000-446). Antigen retrieval was performed by heating the cells to 90 degrees Celsius in 0.1 mM EDTA (Ambion AM9260G) in PBS. Cells were then washed and permeabalized with PBS/0.3% Triton X-100 (J.T.Baker X198-07) for 15 minutes. Immunocytochemical staining was then performed as follows. Antigen blocking was performed with 5% normal donkey serum (Jackson ImmunoResearch 017-000-121) in PBS for an hour. Staining was performed by overnight incubation with primary antibody at 4 degrees Celsius. For the primary screen, PDX-1 antibody (R&D AF2419) was used to reveal beta-cells and ki-67 antibody (BD Bioscience 556003) to visualize proliferating cells. Without wishing to be bound by theory, the failure of prior beta-cell replication screens is primarily a consequence of using a cytoplasmic marker (Insulin) as the β-cell identifier. The use of a cytoplasmic marker prevents accurate attribution of nuclear replication to a specific cell identity i.e., in dense culture it is impossible to attribute a cytoplasm to a specific nucleus given the proximity to multiple nuclei. Additional Immunohistochemistry antibodies included Phosphohistone 3 (Millipore 06-570), Insulin (Dako A0564), Glucagon (Millipore 4031-01F) and Vimentin (Millipore ab5733). For western blotting anti-bodies included ADK (Abcam ab38010, ab64825) and RAN (BD Bioscience 610340) as a loading control.

Once compound-treated islet cells were stained, β-cell replication was assessed via automated image acquisition and analysis using a Cellomics ArrayScanVTI. The acquisition thresholds/parameters were established such that the computer-based calls of replication events were consistent with human-based calls. The parameters used were directed at the identification of (1) PDX-1+ cells and (2) ki-67 positive staining within the PDX-1 positive area. The algorithm was a three step process. First the nuclear area was predicted by the software based upon the expected PDX-1 staining pattern. Second PDX-1 positive cells (selected objects) were counted based upon the average staining intensity within the predicted nuclear area (Note: total signal intensity was essentially equal in quality but was not typically used). Third, the number of PDX-1 positive cells (selected objects) that simultaneously were Ki-67 positive was determined by determining the average fluorescence intensity of ki-67 staining within the predicted nuclear area (again, total intensity was also occasionally used with equal efficacy). The specific thresholds (fluorescence intensities) varied from experiment to experiment. In general, the basal replication rate was set at roughly 1% i.e., the algorithm was varied until 1% of DMSO treated PDX+ cells were reported to be double positive for PDX-1 and ki67. The high content libraries that were screened included a kinase inhibitor library (approximately 300 compounds), a cannabinoid library (80 compounds), a hormone library (80 compounds) and a phosphodiesterase inhibitor library (40 compounds). In total approximately 500 compounds were tested. Hit compounds were subsequently purchased (A10, 5-iodotubercidin (Calbiochem 407900); ABT-702 (B8, Sigma A2721)) and confirmed in dose curves and other studies. In the screening assay, a 2.3-fold induction of β-cell replication was seen for compound corresponding to A10 (data not shown). Although compounds D7 and F3 also showed more than 2 standard deviation increase in fluorescence, these two compounds were observed to be auto-fluorescing.

### Example 2

### PH3 Induction by A10 and B8

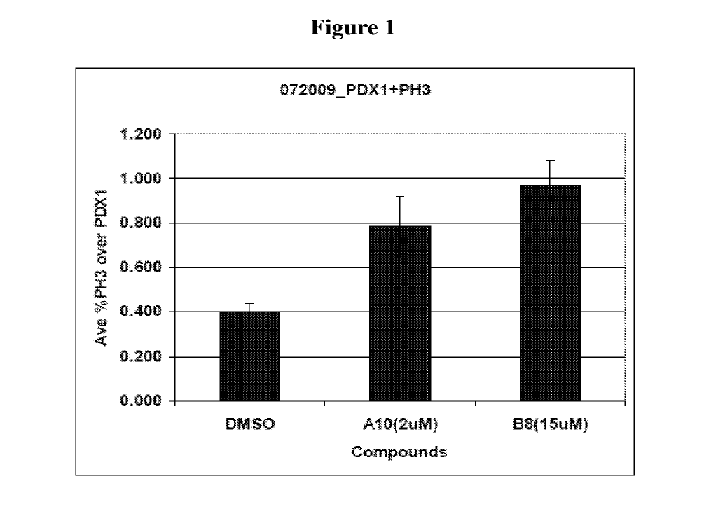

Using a protocol similar to as described in Example 1, β-cells were treated with A10 (2 μM), or B8 (15 μM). Phosphohistone 3 antibody (Millipore 06-570) was used to visualize proliferating cells. As can be seen in FIG. 1, both A10 and B8 increased ratio of PH3 over PDX-1 relative to a control DMSO treatment.

### Example 3

### A10 Specifically Increases Replication of β-Cells

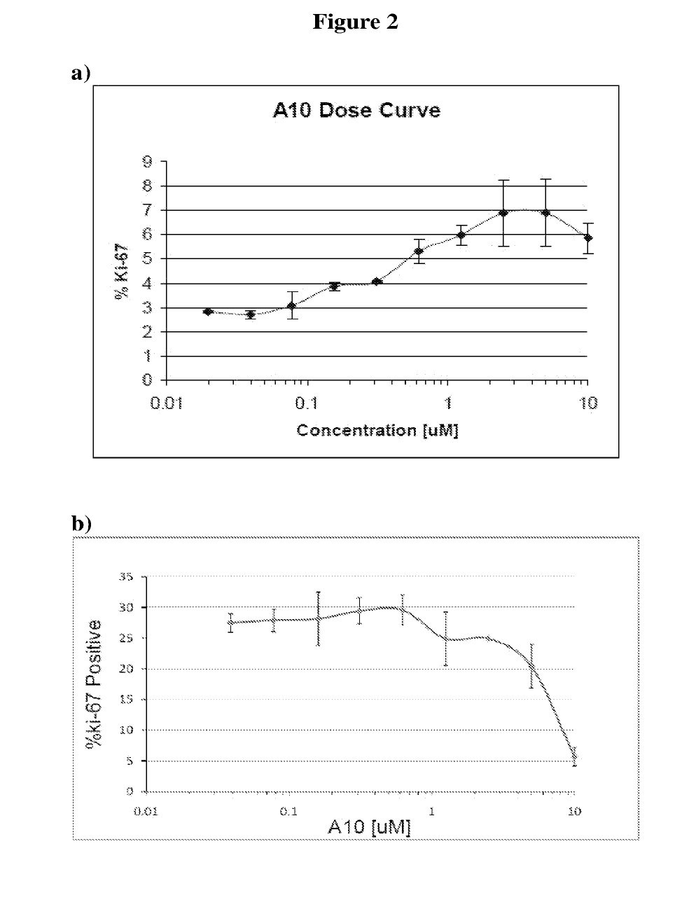

Using a protocol similar to as described in Example 1, β-cells or mouse dermal fibroblasts were treated with 10 uM, 5 uM, 2.5 uM, 1.25 um, 0.625 uM, 0.3125 uM, 0.156 uM, 0.078 uM, 0.04 uM, or 0.019 uM of A10. As seen in FIG. 2a, % of Ki-67 positive β-cells were seen to increase in a dose dependent manner. However, as seen in FIG. 2b, % Ki-67 positive mouse dermal fibroblasts did not change on treatment with A10. These results are consistent with the little or no ADK expression in mouse fibroblasts relative to mouse islets as seen from Western blot analysis (data not shown).

### Example 4

### Dose Curve of B8

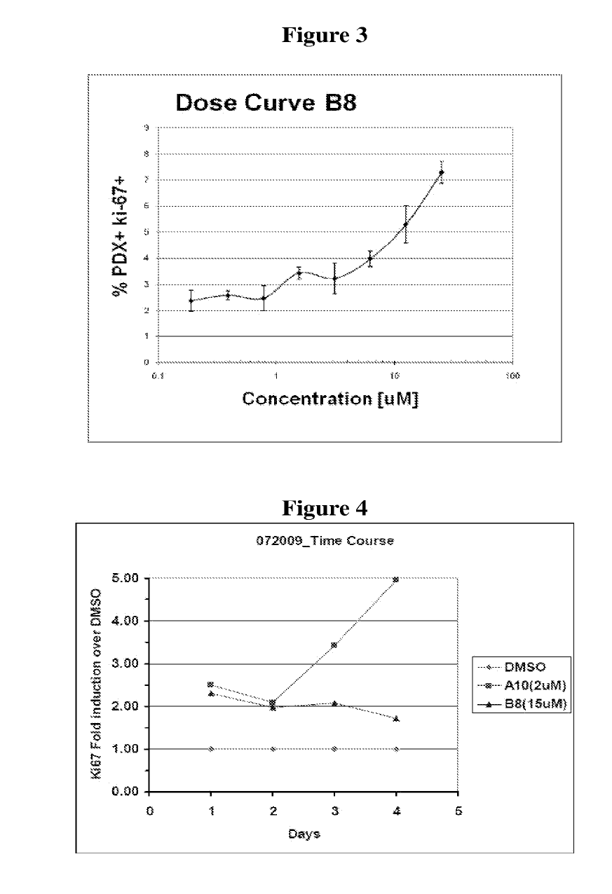

Using a protocol similar to as described in Example 1, β-cells o were treated with 30 uM, 15 uM, 7.5 uM, 3.75 uM, 1.875 uM, 0.94 uM, 0.47 uM, or 0.23 uM of B8. As seen in FIG. 3, % of PDX-1 and Ki-67 positive cells increased in a dose dependent manner on treatment with B8.

### Example 5

### Time Course of A10 and B8 Treatment

Using a protocol similar to as described in Example 1, β-cells were treated with A10 (2 μM) or B8 (15 μM). Induction of Ki-67 relative to DMSO control was measured at different time points. Compounds were added on day 0 and media was changed on day 2. Results are shown in FIG. 4.

### Example 6

### Effect of Serum Concentration on A10 Treatment

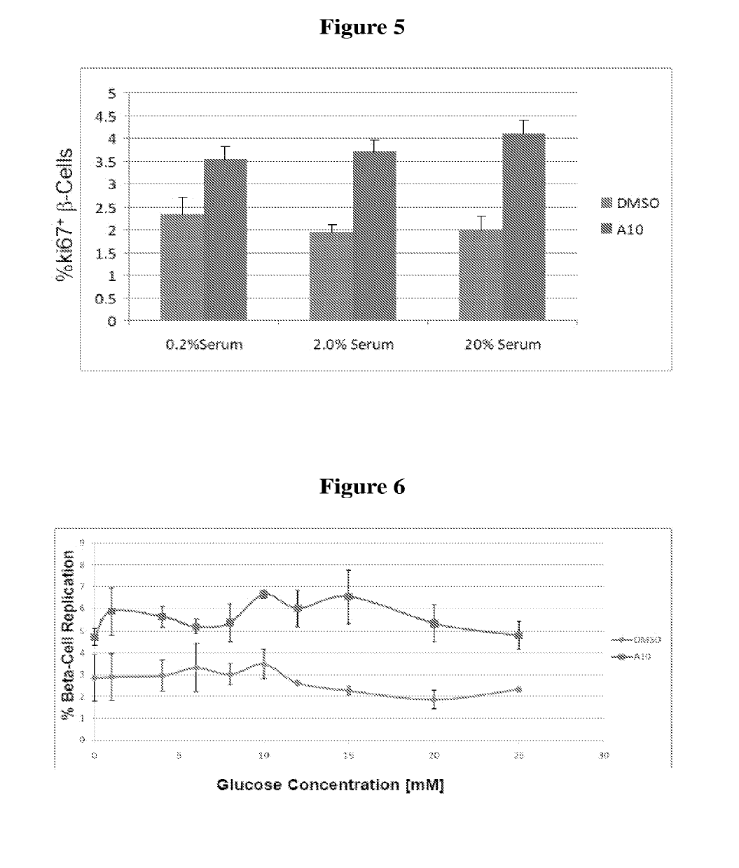

Using a protocol similar to as described in Example 1, β-cells were treated with A10 (2 μM) in culture media comprising different 0.2%, 2.0%, or 20% of serum. As seen in FIG. 5, in the A10 treated samples, % Ki-67 positive cells increased with increasing serum concentration. On the other hand, in the DMSO treated samples, % Ki-67 positive cells decreased with increasing serum concentration.

### Example 7

### Effect of Glucose on A10 Treatment

Using a protocol similar to as described in Example 1, β-cells were treated with 2 μM of A10 or DMSO in culture media comprising 25 mM, 20 mM, 15 mM, 12.5 mM, 10 mM, 8 mM, 6 mM, 4 mM, 1 mM, or 0 mM of glucose. As seen in FIG. 6, glucose concentration had little effect on % beta-cell replication in the A10 or DMSO treated samples.

### Example 8

### Effect of Adenosine on β-Cell Replication

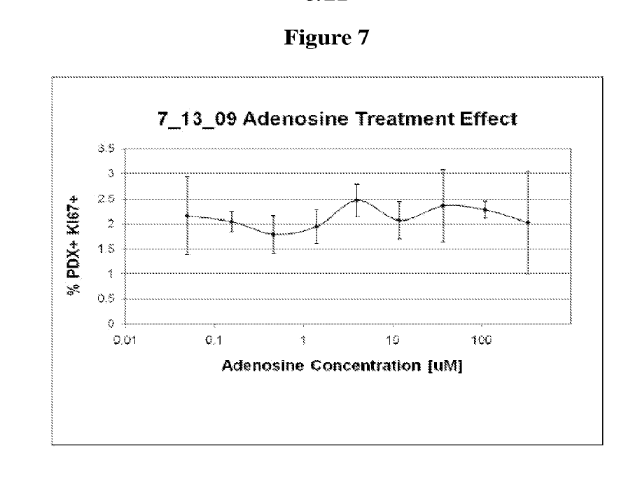

To test it A10 effect on β-cell replication was due to an increase in adenosine concentration in the cells rat islets were treated with 1000 uM, 330 uM, 110 uM, 37 uM, 12 uM, 4 uM, 1.4 uM, 0.46 uM, 0.156 uM, or 0.05 uM of adenosine. As seen in FIG. 7, adenosine had little effect on % PDX-1 and Ki-67 expressing cells.

### Example 9

### Effect of Adenosine Receptor Antagonists on β-Cell Replication Enhancement with A10

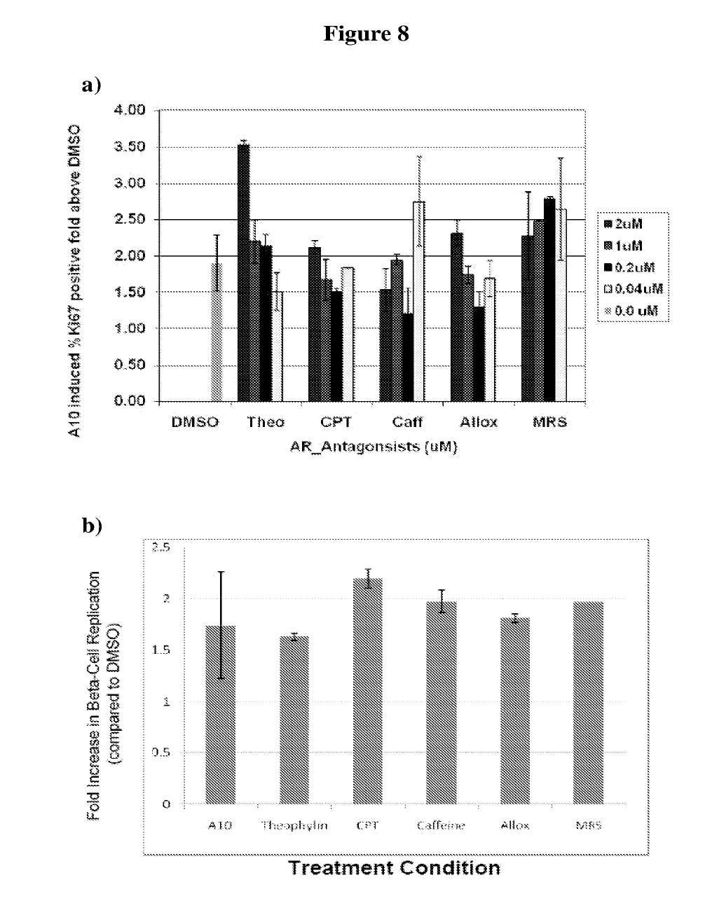

To test if A10 effect was mediated by adenosine receptor β-cells were treated with 5 uM, 1 uM, 0.2 uM, or 0.04 uM of A10 or DMSO in culture media comprising different concentrations of adenosine receptor antagonists Theophylin, CPT, caffine, Allox, and MRS. Results are shown in FIGS. 8a and 8b. As seen in FIG. 8b, adenosine antagonists at 0.2 μM had little effect on beta-cell replication fold increase with A10 treatment.

### Example 10

### AMPK Activation

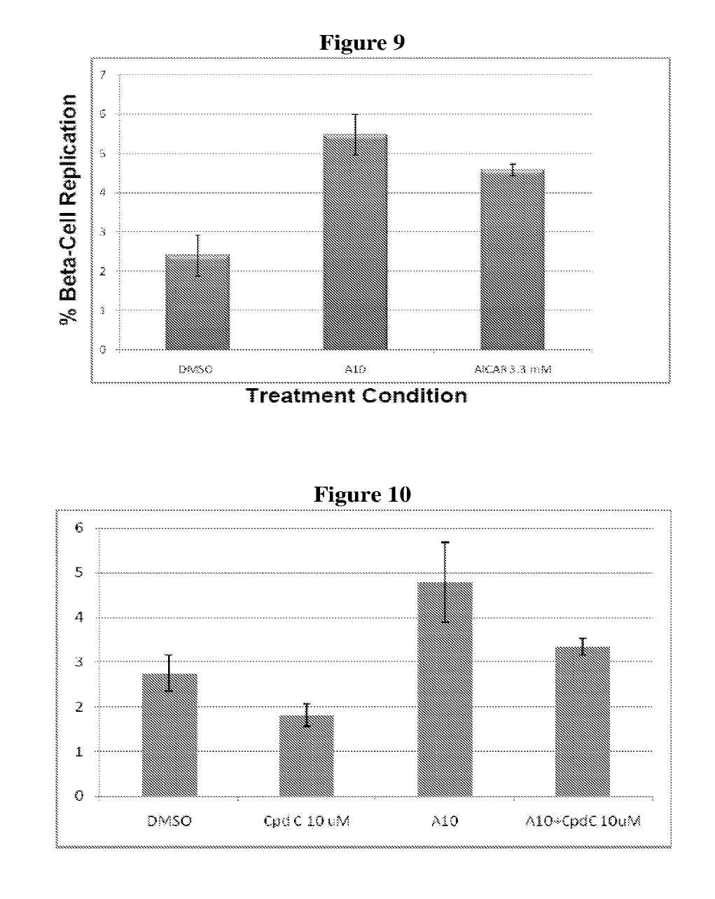

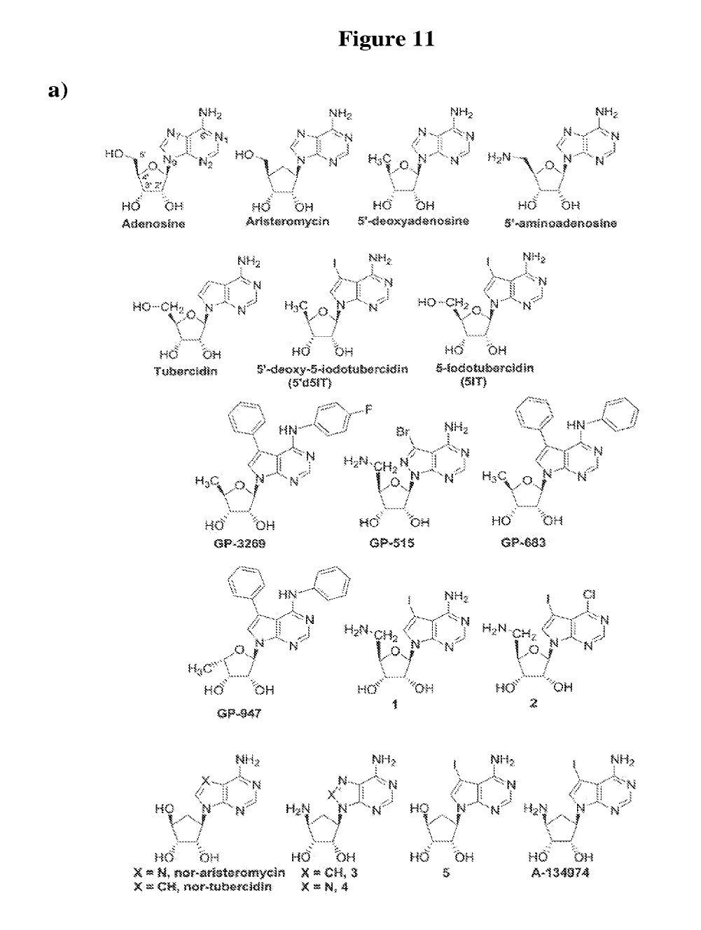

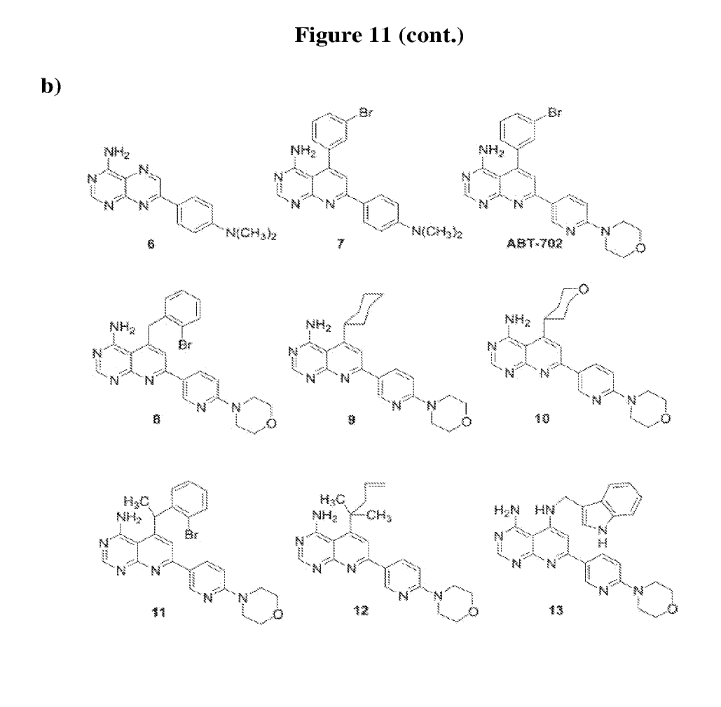

It was postulated that A10 leads to increase in β-cell replication by indirectly activating AMPK. To test this, β-cells were treated with A10 (2 μM), AMPK activator AICAR (3.3 μM). As seen in FIG. 9, activation of AMPK with AICAR lead to an increase in % β-cell replication.

### Example 11

### Effect of AMPK Inhibition on β-Cell Replication Enhancement with A10

Effect of AMPK inhibition with Cpd C was tested on β-cell replication enhancement with A10. As seen in FIG. 10, inhibition of AMPK with Cpd C (10 μM) lead to a decrease in beta-cell replication relative to the uninhibited controls.

### Example 12

### In Vivo β-Cell Replication

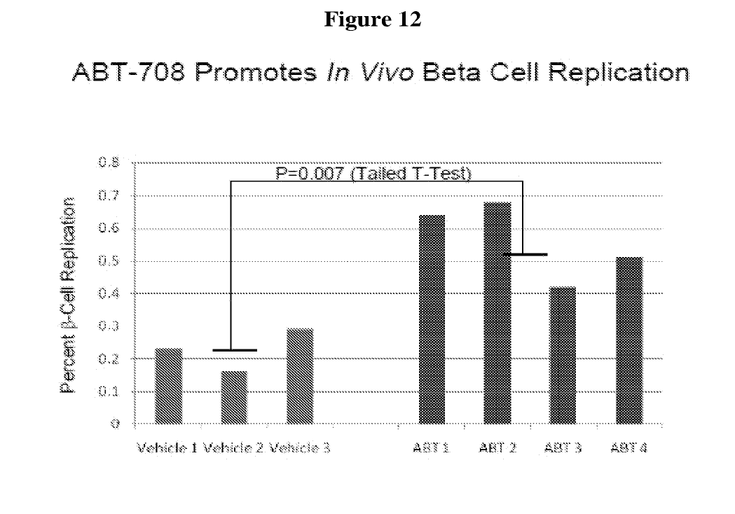

12-week old C57/B6 female animals were injected BRDU 10 ul/g (Sigma B5002) and either with ABT-702 (21 mg/kg) or DMSO vehicle. 24-hours post treatment, animals were sacrificed and the pancreas was fixed, sectioned and stained for PDX-1 and BRDU (Amaersham RPN202). Every fourth section was included in analysis and a minimum of 2000 PDX-1⁺ per animal were counted. Results are shown in FIG. 12.

### Example 13

### Adenosine Kinase Inhibition Selectively Promote Islet β-Cell Replication

Islet Isolation and Primary Screen Protocol:

Rat and mouse islets were isolated as previously described. (M. Gotoh, et al., Transplantation 43, 725-730 (1987)) All animal work was approved and carried out in accordance with our institutional animal care and use committee. Porcine islets were provided by VitaCyte (Indianapolis, Id.). Islets were incubated (37° C., 5% CO₂) overnight in islet media (Mediatech 99-786-CV; 10% FBS serum (Valley Biomedial BS3033); 8.3 mM Glucose (Sigma G7528); 1× Penicillin/Streptomycin (Invitrogen 15070-063); 1× Glutamax (Invitrogen 35050-079)). The following morning, islets were trypsinized into cellular clusters of 1-3 cells, re-suspended in islet media and plated into the wells of a 96-well plate (Sigma CLS3904) that had been coated with 804G (a rat bladder carcinoma cell line) conditioned media. The cellular plating density was 60 k cells/well and greater than 95% viability was confirmed at the time of plating. The islet cells were allowed 48-hours to adhere at which time the media was changed (as above, except 2% serum, 5 mM glucose) and the cells were compound treated. For screening, compounds were tested at 1 μM and 10 μM concentrations in duplicate on a single occasion. After 24-hours of compound treatment, cells were fixed with fresh 4% PFA. Antigen retrieval was performed by heating the cells to 90 degrees Celsius in 0.1 M EDTA in distilled water. Cells were then washed and permeabalized with PBS/0.3% Triton X-100 (J.T.Baker X198-07) for 15 minutes. Immunocytochemical staining was then performed as follows. Antigen blocking was performed with 5% normal donkey serum (Jackson ImmunoResearch 017-000-121) in PBS for an hour. Staining was performed by overnight incubation with primary antibody in PBS at 4 degrees Celsius. For the primary screen, PDX-1 antibody (R&D AF2419; 1:300) was used to reveal beta-cells and ki-67 antibody (BD Bioscience 556003, 1:300) to visualize proliferating cells.

β-cell replication was assessed via automated image acquisition and analysis using a Cellomics ArrayScanVTI. The acquisition thresholds/parameters were established such that the computer-based calls of replication events were consistent with human-based calls. The high content libraries that were screened included a kinase inhibitor library (approximately 300 compounds), a cannabinoid library (80 compounds), a hormone library (80 compounds), a phosphodiesterase inhibitor library (40 compounds) and a portion of the NIH Clinical Collection (250 compounds). All organic compounds were suspended in DMSO and used at a minimum dilution of 1:1000. Subsequently, compounds were purchased from a variety of vendors: 5-iodotubercidin (Calbiochem 407900); ABT-702 (Sigma A2721; Tocris); Aristeromycin (Sigma A0928) and dideoxy-7-iodo-deazadenosine (chem-ipex 15132). β-cell replication experiments with Exendin-4 (Sigma E7114) and GLP-1 (AbCam, ab50245) were performed as described above. β-cell replication experiments with glucose were performed as above with modification of the glucose concentration and culture duration. In all extended cultures, the media was changed every 48 h.

Immunohistochemistry:

A variety of cell populations were identified by immunohistochemical markers: β-cell (Guinea pig anti Swine Insulin; Dako, A0564); α-cells (Guinea pig anti Glucagon; Millipore, 4031-01F); PP-cells (Rabbit anti Pancreatic Polypeptide; Pierce, PAI-36141); δ-cells (Rabbit anti Somatostatin; Dako, A0566) and fibroblasts (Chicken anti Vimentin; Millipore, AB5733). Adenosine Kinase (ADK) with N-terminal specific antibody (Rabbit pAB to ADK; Abeam, ab38010-100) was used for adenosine kinase immunofluorescence. The ADK antibody used for immunofluorescence was validated by transient transfection of Cos-7 cells (ATCC; CRL-1651) with an ADK cDNA (Invitrogen;). Additional antibodies included Phosphohistone 3 antibody (Millipore 06-570), BRDU

Quantification of β-Cell Number:

To quantify the number of β-cells at 0 h, 96 h and 144 h of culture, cells were plated in 96-well plates as above in the presence of vehicle only or 5-IT and 15 mM glucose; final DMSO dilution was 1:1000 in all wells. Time Oh was after cells had been allowed 48 h to adhere. Each condition was represented by 16 separate wells per plate to accommodate small fluctuations in plating density. Media changes were performed every 48 h. Cells were fixed at the appropriate time point and stored to be stained simultaneously for PDX-1. Automated cell counting was performed using the Cellomics ArrayScanVTI which identified PDX-1⁺ cells.

BRDU Assay:

Islet cells were plated as above and allowed 48 h to adhere. The media was changed and included BRDU and DMSO or 5-IT (2 μM) or ABT-702 (15 μM). After 48 h, the cells were washed and regular media was added. Cultures were allowed another 48 h of growth before being fixed. Antigen retrieval was performed by heating to 90° C. for 10 minutes in 0.1M EDTA. Staining was performed using PDX-1 immunofluorescence (as above) to identify β-cells and a BRDU staining kit (Amersham, RPN20) to identify replicating cells. Quantitative image analysis was performed using a Cellomics ArrayScanVTI to count the percentage of PDX-1 cells that were also BRDU

ADK Knock-Down:

The Block-iT polII miR RNAi expression system with EmGFP (Invitrogen, K4938-00) was used for lentiviral knock-down of ADK. Viral concentration was performed using the Fast-Trap kit (Millipore, FTLV00003). Several ADK-directed miRNAs were tested using stable lentiviral transduction of the rat H4IIE hepatoma cell line (ATCC, CRL-1600) followed by evaluation of ADK levels by western blot. The most effective sequence (Top:5′-TGCTGAAGAACTGGCTAATGAACGGTGTTTTGGCCACTGAC TGACACCGTTCAAGCCAGTTCTT (SEQ ID NO: 1); Bottom: 5′-CCTGAAGAACTGGCTTGAACGGTGTCAGTCAGTGGCCAAAACACCGTTCATTAGC CAGTTCTTC (SEQ ID NO: 2)) was selected for use in islet cell experiments. The negative control sequence was (Top: 5′-TGCTGGAAATGTACTGCGCGTGGAGACGTTTTGGCCACTGACTGACGTCTCCACG CAGTACATTT (SEQ ID NO: 3); Bottom: 5′-CCTGGAAATGTACTGCGCGTGGAGACGTTTTGGCCACTGACTGACGTCTCCACGC AGTACATTTC (SEQ ID NO: 4)). Dispersed rat islet cell cultures (as above) were transduced in a 96-well format starting 48-hours after plating. Concentrated virus was used to serially infect islet cultures over the course of 48 h followed by a 4 day culture period prior to cell fixation. The Cellomics ArrayScanVTI was used to quantify fluorescence intensity and determine a variety of parameters: viral transduction efficiency (DAPI⁺,GFP⁺), the percentage of ADK expressing cells (DAPI⁺,ADK⁺), the replication rate of infected β-cells (GFP⁺, PDX-1⁺, ki-67⁺) and uninfected β-cells (GFP⁻, PDX-1⁺, ki-67⁺). ADK expression was determined by western blot using a C-terminal specific antibody to detect both the long and short isoform (Abgent, AP7091b, 1:100).

Hepatocyte Isolation and Proliferation:

Hepatocytes were isolated using a two step collagenase perfusion technique as previously described and cultured in William's E medium supplemented with 10% FBS, hEGF (40 ng/ml; R&D Systems 236-EG-200)) and mHGF (20 ng/ml; R&D Systems 2207-HG-025). (P. O. Seglen, Methods in cell biology 13, 29-83 (1976)) Cultures were compound treated overnight as indicated above and then fixed and stained. Total cell number was assessed with DAPI and replicating cells were ki-67⁺. The Cellomics ArrayScanVTI was used for image acquisition and analysis.

Quantitation of In Vivo Replication:

12-week old C57/B6 female animals were injected with BRDU (Sigma B5002; 10 ul/g) and with either ABT-702 (21 mg/kg) or DMSO vehicle. 24-hours post treatment, the animals were sacrificed and the relevant organs were harvested. All experiments were performed with a minimum of 4 animals per treatment group. Every fourth 12 μM section was used for analysis and a minimum of 2,000 β-cells, exocrine cells and hepatocytes per organ per animal were counted. Analysis was performed by manual picture acquisition and cell counting. β-cells were identified by either PDX-1 staining or insulin staining with similar results. Exocrine cells were approximated by counting all nuclei outside the islet structure. A minority of these cells were not exocrine cells. Hepatocytes were identified as DAPI⁺ albumin⁺ cells (Bethyl Laboratories, A90-234A). Dividing cells were BRDU⁺ (Amaersham RPN202).

Statistic:

The data are presented as the mean of multiple replicates (minimum of 4 wells per data point shown) performed simultaneously. All of the presented experimental results were repeated greater than twice. Error bars show the standard deviation unless otherwise specified. Results were compared using the two-tailed t test. The EC50 was calculated using nonlinear regression with the top replication rate constrained.

### Results

Development and Performance of a High-Throughput Primary β-cell Replication Assay:

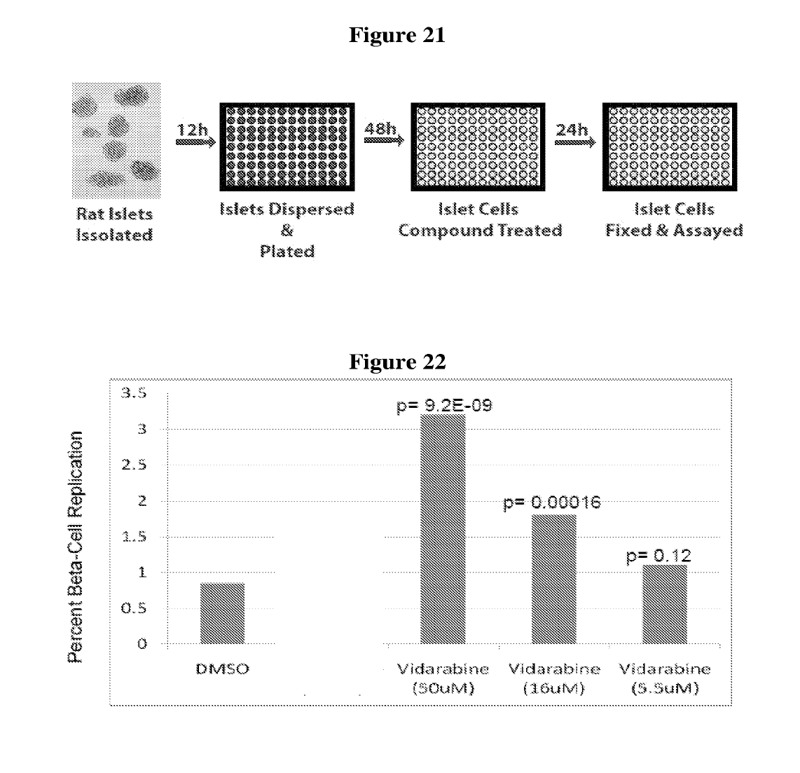

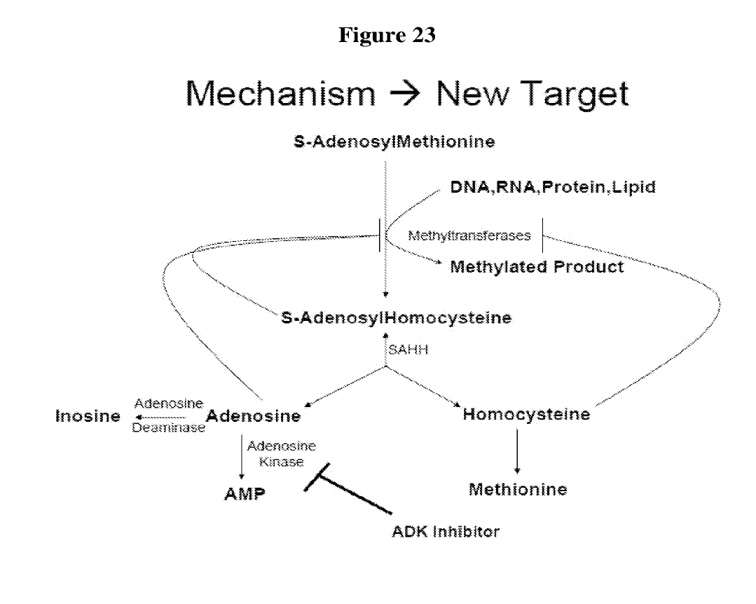

To identify compounds that increase β-cell replication the invention provides a screening platform using freshly isolated rat islet cells (FIG. 21). Although the use of primary cells limits the supply of β-cells and might be expected to introduce behavioral variability, this approach maximizes retention of in vivo metabolic characteristics which are pertinent to the mitotic behavior of β-cells. (Y. Zimmer, et al., FEBS letters 457, 65-70 (1999)) These dispersed cultures contained ˜75% β-cells (PDX-1⁺), ˜18% α-cells (glucagon⁺), ˜3% fibroblasts (vimentin⁺) and ˜5% other cell types. Dispersed rat islet cells were immunofluorescently stained for the β-cell transcription factor PDX-1, alpha cell hormone glucagon, fibroblast marker vimentin and DNA in combination (data not shown). Percentages were obtained by automated image acquisition and analysis (n=80 images from a representative rat islet preparation). The standard deviation for all values was less than 10%. PDX-1 positive cells expressed either insulin or somatostatin (data not shown). The analysis of multiple cell populations in these primary islet cultures allows one to check for β-cell specificity in the replication assay.

Isolated islets were recovered overnight in a tissue culture incubator prior to being dispersed and plated the following day. To allow cell adhesion, islet cells were incubated for 48 hours prior to addition of fresh media and compound treatment. After 24 hours of compound treatment, the cells were fixed, stained and assayed. PDX-1 is a transcription factor expressed by β-cells and δ-cells. (P. Serup, et al., The Biochemical journal 310 (Pt 3), 997-1003 (1995)) Among the PDX-1 population, >90% of the cells are insulin⁺ β-cells (data not shown) (J. Suckale, et al., Front Biosci 13, 7156-7171 (2008)). Nuclear PDX-1 staining was used as the primary β-cell marker because islet-cells grow in dense irregular clusters that cause a cytoplasmic stain, such as insulin, to be ambiguously associated with multiple nuclei. As a result of this ambiguity, ki-67⁺ nuclei from rapidly replicating cells such as fibroblasts have the potential to be incorrectly attributed to insulin cells. The basal in vitro β-cell replication rate showed moderate inter-experiment variability (0.4-3.5%), and was typically higher than the in vivo β-cell replication rate (0.8±0.2%) of similar aged animals as determined by the percentage of PDX-1⁺ cells that co-expressed ki-67.

ADK-Is Promote β-Cell Replication:

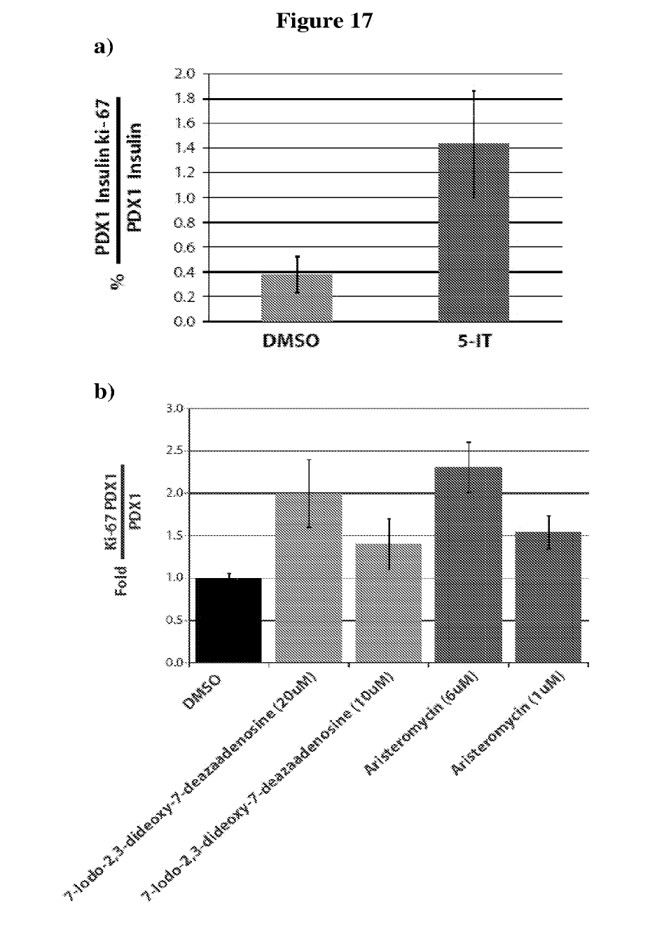

Approximately 750 compounds from a carefully selected library of cell permeable bioactive compounds were screened for their ability to increase β-cell replication. By using PDX-1 immunostaining to identify β-cells and ki-67 immunostaining to identify dividing cells, replicating PDX-1⁺ cells were easily distinguished from dividing PDX-1⁻ cells by the co-localization of PDX-1 and ki-67 (data not shown). Two compounds were identified; both well characterized adenosine kinase (ADK) inhibitors (ADK-Is). 5-iodotubercidin (5-IT; CAS 24386-93-4) and ABT-702 (CAS 214697-26-4), increased the percentage of dividing β-cells 2-3 fold above the background after 24 hours, and have a significant effect on β-cell number (see below). Independent of the variable baseline β-cell replication rate, the ADK-Is consistently caused a 2 to 3-fold increase in the β-cell replication rate. In the displayed experiment, the hit compounds increased the β-cell replication rate from ˜2.5% to ˜6.5%. To confirm our results using PDX-1 to identify β-cells, we performed similar experiments using PDX-1 and insulin co-staining to identify β-cells (FIG. 17a).

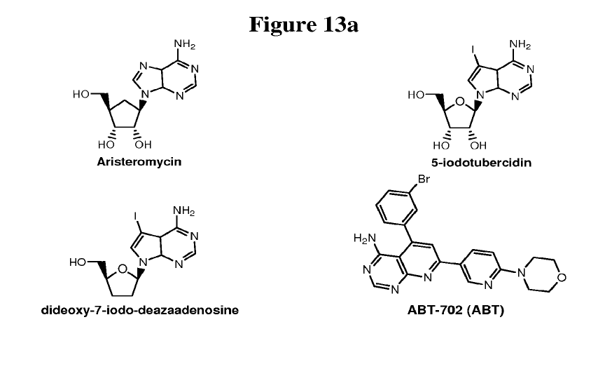

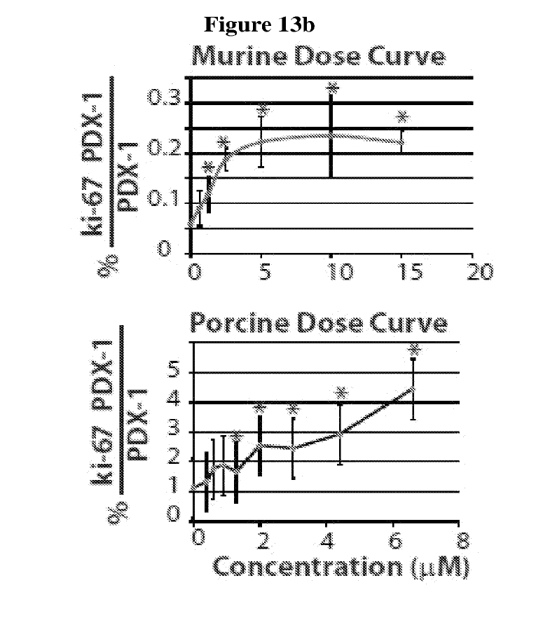

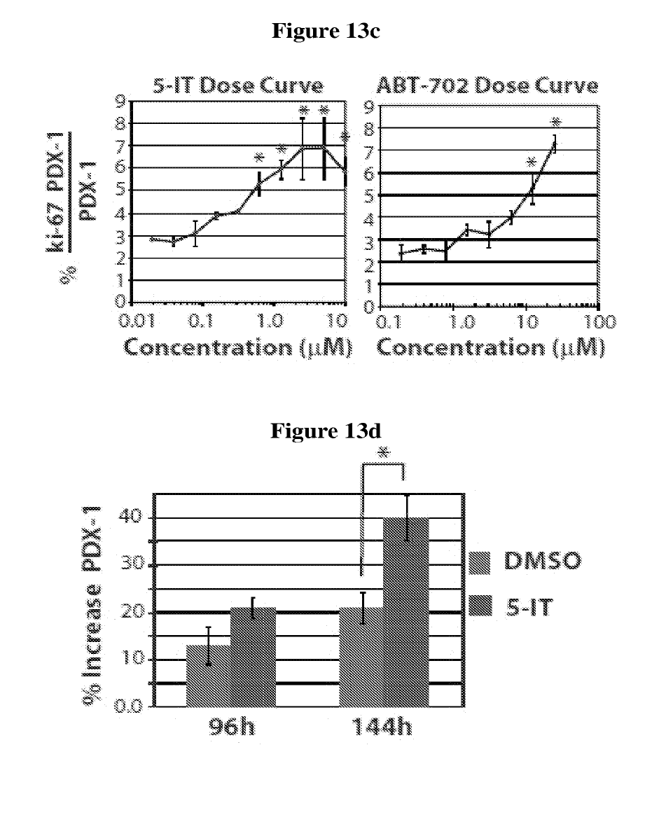

Additional ADK-Is for their ability to promote β-cell replication were tested next. Two additional ADK-Is demonstrated similar efficacy to the primary hit compounds (FIG. 17b) and are shown in FIG. 13a. (E. A. Kowaluk and M. F. Jarvis, Expert opinion on investigational drugs 9, 551-564 (2000)) The effect of ABT-702 on murine and porcine islets was also tested. Healthy cultures of dispersed islet cells from these species were established and the same antibodies previously used for the rat islet cell cultures effectively identified both β-cells (PDX-1) and replicating cells (ki-67) (data not shown).

Compound treatment of islet cells from both of these species caused an induction of β-cell replication similar to that observed for rat islet cells, albeit with somewhat different potencies (FIGS. 13b and 13c). Both compounds demonstrated increasing efficacy with increasing dose. Although 5-IT was more potent than ABT-702 both compounds had a maximum induction of ˜2-3-fold. One notable observation is that the analysis of ABT-702 at concentrations above ˜20 μM was limited by background fluorescence.

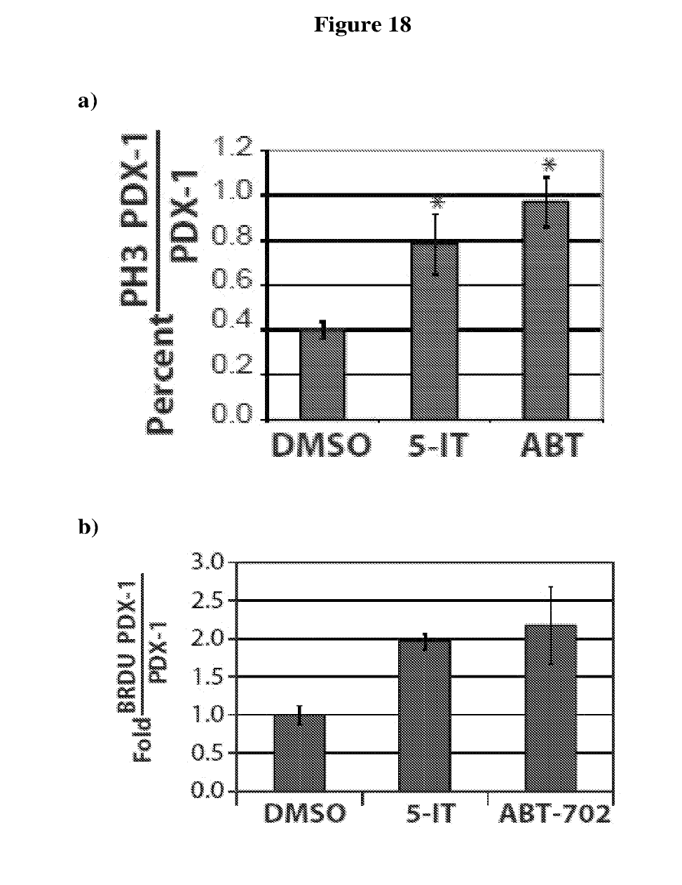

The replicative effect of these compounds was confirmed by assessing the percentage of β-cells that expressed the mitotic phase marker phosphohistone-H3 (PH3) in response to compound treatment (data not shown). Using PH3 a similar 2 to 3-fold-increase in β-cell replication was observed (FIG. 18a). In addition, a pulse-chase experiment was performed using BRDU to mark S-phase cells. Islet cells were cultured in the presence of BRDU and vehicle or compound for two days followed by a two day wash-out period. Islet cultures treated with the ADK-Is demonstrated a 2-fold increase in the percentage of β-cells that incorporated BRDU cells (FIG. 18b). The ability of the ADK-Is to cause a similar induction of a broad cell cycle marker (ki-67) and narrow mitotic phase marker (PH3) as well as increase BRDU incorporation all suggest that these compounds increase mitotic activity.

To test whether ADK-Is can increase the absolute number of β-cells, islet cells were cultured in the presence of 5-IT or DMSO for several days and then the total number of β-cells was counted and compared to the number of β-cells present at time 0. Only a small difference in β-cell number was evident after 96-hours, but there was a sizable increase in β-cell number after 144-hours (FIG. 13d). At day 6, the number of β-cells in the 5-IT-treated wells had increased by 40% compared to a 20% increase in the control wells. The increase which resulted from ADK-1 treatment was consistent with a 2-fold increase in β-cell replication and suggests a change from a basal replication rate of ˜3% per day to ˜6% per day in our cultures.

ADK is Expressed by β-Cells and Negatively Regulates β-Cell Replication:

ADK is a member of the sugar kinase group of enzymes, composed of three metabolic families (hexokinases, ribokinases and galactokinases) which play important roles in cellular metabolism. (P. Bork, C. Sander and A. Valencia, Protein Sci 2, 31-40 (1993)) ADK is a ribokinase that regulates the intracellular and extracellular adenosine levels through its ability to catalyze the phosphorylation of adenosine to AMP using ATP as the phosphate donor. (T. A. Krenitsky, R. L. Miller & J. A. Fyfe, Biochemical pharmacology 23, 70-72 (1974) and J. Park &R. S. Gupta, Cell Mol Life Sci 65, 2875-2896 (2008)). While this enzyme is broadly expressed, it is highly expressed in the liver and pancreas. (M. Andres & I. H. Fox, The Journal of biological chemistry 254, 11388-11393 (1979)) ADK has two known forms, a long nuclear isoform and a short cytoplasmic isoform. (X. A. Cui, et al., Biochemical and biophysical research communications 388, 46-50 (2009)) The cytoplasmic form participates in the purine salvage pathway, whereas the long form is a global regulator of methyltransferase reactions via adenosine's feedback regulation of S-adenosylhomocysteine hydrolase activity. (N. M. Kredich & D. V. Martin, Jr., Cell 12, 931-938 (1977) and Boison, L. et al., Proceedings of the National Academy of Sciences of the United States of America 99, 6985-6990 (2002)) Thus, inhibition of ADK has the dual effect of increasing extra-cellular adenosine and inhibiting methyltransferase reactions.

ADK immunostaining revealed nuclear expression of ADK in β-cells (data not shown). In contrast, ADK staining was in the cytoplasm, not the nucleus, in fibroblasts and α-cells (data not shown). Although ADK localization in δ-cells (somatostatin⁺-cells) was variable, ADK was generally present in the nucleus of these cells (data not shown). To ensure that staining was specific, antibody used was validated by transient transfection of COS cells with a full length ADK cDNA which demonstrated strong nuclear expression pattern and confirmed the specificity of our islet-cell staining (data not shown). The presence of nuclear staining in β-cells, but not α-cells, suggests that the long form of ADK is expressed in β-cells and not α-cells.

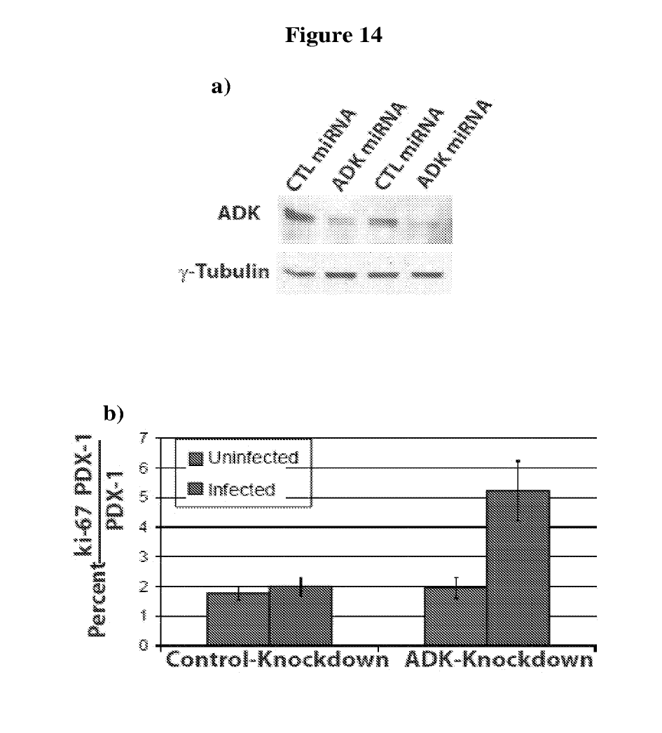

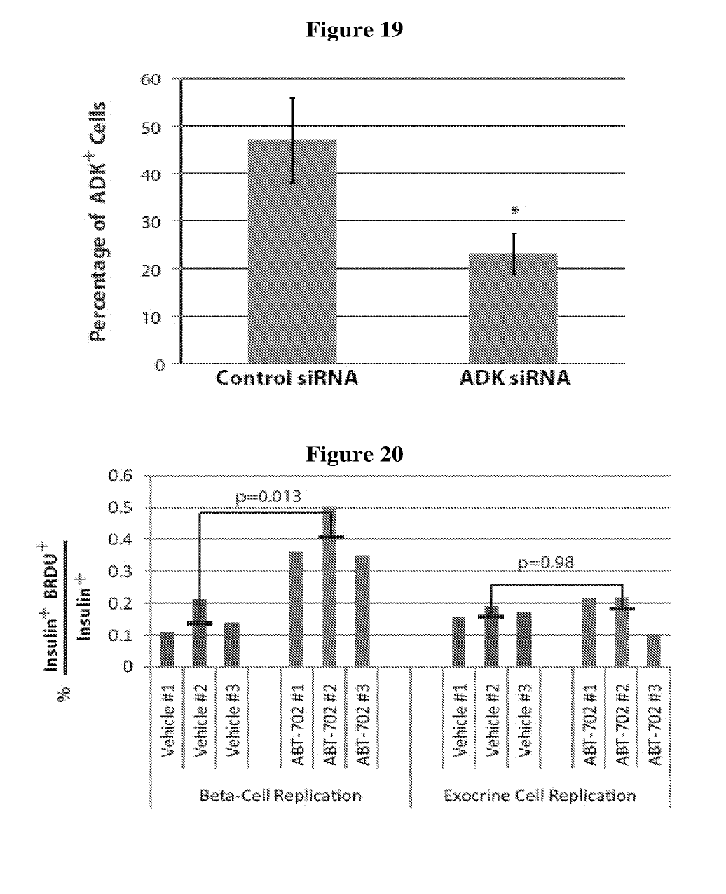

Whether the ADK in β-cells acts as a negative regulator of replication was tested next. We used lentiviral infection to direct the expression of GFP and either a non-specific inhibitory RNA (siRNA) or one that was targeted to ADK. The ability of the ADK-directed siRNA to knock-down ADK protein levels was confirmed by western blot and immunostaining (FIGS. 14a and 19). By infecting approximately half of the islet cell culture, as determined by GFP expression, the β-cell replication rate of infected and non-infected cells within the same well could be separately analyzed. It was reasoned that if ADK acts as a cell autonomous regulator of β-cell replication then β-cells that receive the negative control plasmid and β-cells that remained uninfected would all have the same replication rate whereas β-cells infected with the ADK-targeted siRNA virus would have an increased replication rate. Indeed, the results confirmed this prediction (FIG. 14b). The uninfected β-cells and the control infected β-cells all had the basal proliferation rate of ˜2%. In contrast, β-cells that received the ADK-directed siRNA demonstrated a 2.5-fold increase in their replication rate. This result indicated that ADK is a cell-autonomous negative regulator of β-cell replication and can be the molecular target of the ADK-Is.

ADK-Is and Glucose or GLP-1R Agonists have an Additive Effect on β-Cell Replication:

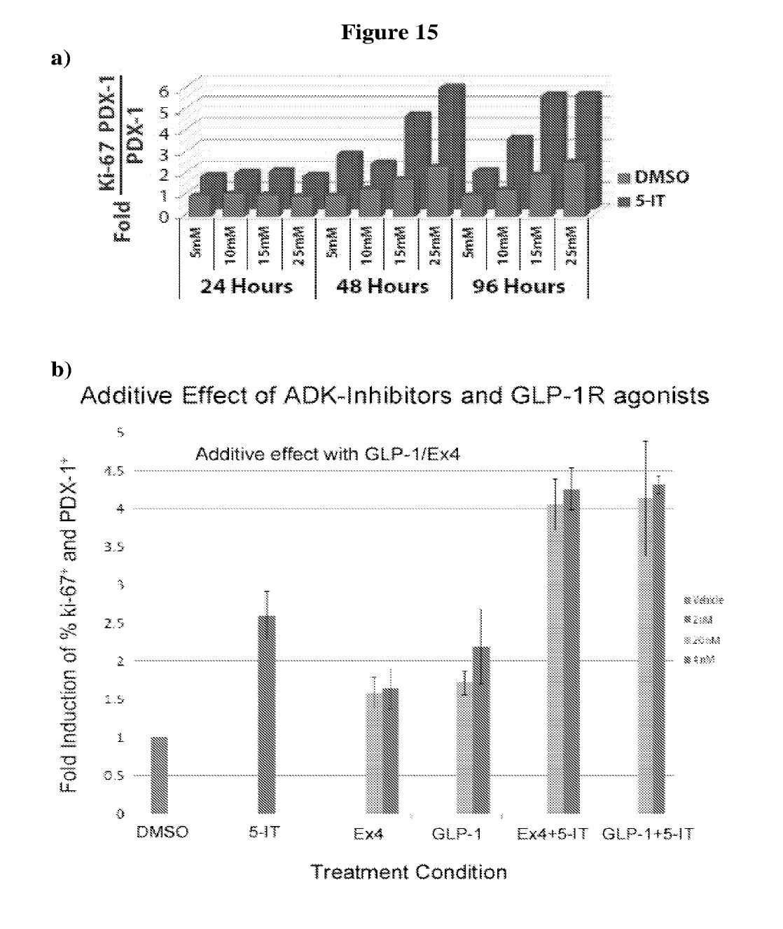

Hyperglycemia is considered to be the primary physiologic driver of β-cell proliferation despite relatively little in vitro evidence to support this widely accepted principle. (S. Bonner-Weir, et al., Diabetes 38, 49-53 (1989) and L. C. Alonso, et al., Diabetes 56, 1792-1801 (2007)) Taking advantage of the β-cell replication platform described herein, it was demonstrated that glucose has a concentration-dependent effect on β-cell proliferation (FIG. 15a). However, the response kinetics appeared to be different from that of AKD-Is. ADK-Is increased ki-67 staining by 24 h whereas an effect of glucose was not seen until later (48 h). Without wishing to be bound by a theory, a distinct mechanism of action for glucose and ADK-Is is supported by the additive effect of 5-IT at all of the tested glucose concentrations (FIG. 15a). The combination of high glucose and 5-IT caused a 5-fold induction of the replication rate above the base-line rate. The results of this experiment indicated that the addition of ADK-Is to a hyperglycemic environment can significantly increase β-cell growth.

GLP-1R agonists have received attention for their ability to promote insulin release, improve glycemic control and increase β-cell replication. (J. H. Nielsen, et al., Diabetes 50 Suppl 1, S25-29 (2001) and D. J. Drucker & M. A. Nauck, Lancet 368, 1696-1705 (2006)) Given that they act by augmenting the β-cell response to glucose, these agonists can also augment the β-cell replication effect of the ADK-Is. Accordingly, rat islet cultures were incubated with GLP-1R agonists (Glucagon-like peptide-1 (GLP-1) or Exenatide-4 (Ex-4)) with and without 5-IT. While the GLP-1R agonists only modestly increased β-cell replication (˜1.5-fold) compared to 5-IT (˜2.5-fold), the addition of 5-IT to the GLP-1R agonists led to an additive replication effect (˜4-fold) (FIG. 15b). These results were consistent with ADK-Is acting in a GLP-1-independent manner and demonstrate the potential for modulating these two pathways in combination to increase β-cell replication.

ADK-Is Selectively Promote β-Cell Replication:

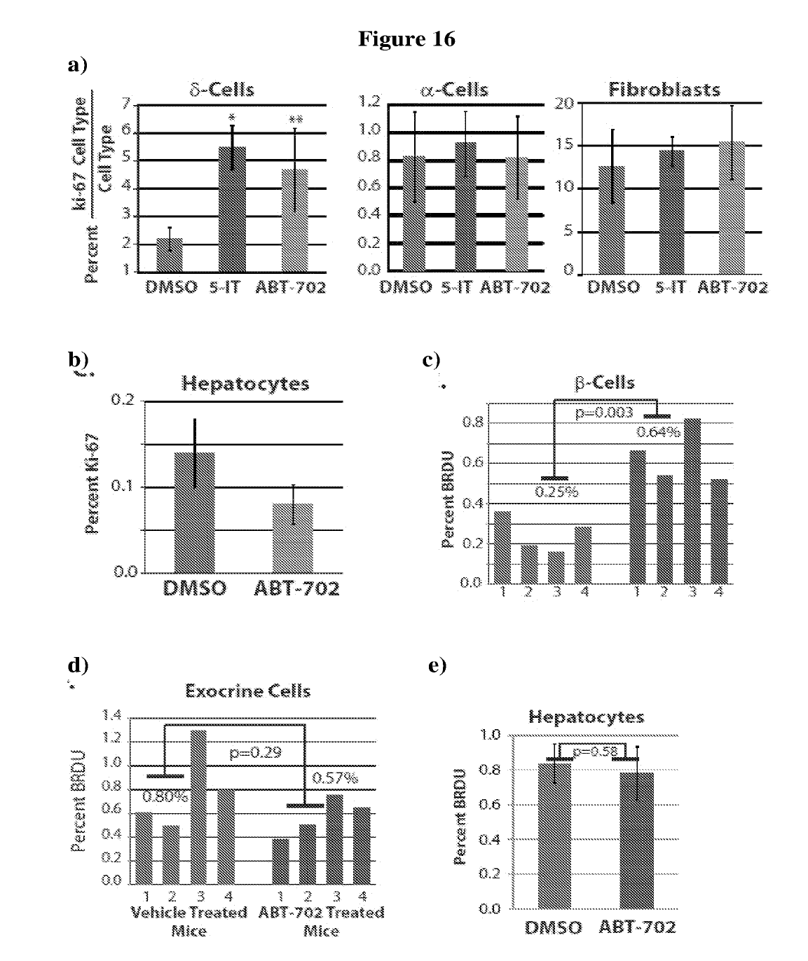

The replication rate of multiple cells was assessed in the islet culture: PP-cells, α-cells, δ-cells and fibroblasts (FIG. 16a). δ-cells can also show an increase replication in response to ADK-Is because like β-cells, δ-cells express nuclear ADK, and share the physiologic property of secreting its hormone in response to glucose. (M. Braun, et al., Diabetologia 52, 1566-1578 (2009)) Indeed, δ-cells did demonstrate a significant increase in replication in response to ADK-I treatment whereas fibroblasts, α-cells and PP-cells do not (FIG. 16a). The replication rate of PP-cells is not shown because their division is extraordinarily rare under the culture conditions used herein.

In addition to β-cells, hepatocytes also express high levels of ADK and therefore can be expected to proliferate in response to ADK-Is. (Boison, L. et al., Proceedings of the National Academy of Sciences of the United States of America 99, 6985-6990 (2002)) To test this possibility, murine hepatocytes were isolated, placed in culture and treated with ABT-702 (5-IT was not well tolerated by the hepatocytes). The replication rate of these cells did not increase in response to drug treatment (FIG. 16b). Therefore, the replication effect of ADK-Is was selective.

ADK-Is Promote β-Cell Replication In Vivo:

Encouraged by the in vitro results, the ability of ABT-702 to selectively promote β-cell replication in vivo was tested. ABT-702 was selected because of its longer half-life compared to 5-IT. (G. Z. Zheng, et al., Bioorganic & medicinal chemistry letters 11, 2071-2074 (2001)) Indeed, a single intraperitoneal injection of ABT-702 resulted in a 2-fold increase in BRDU incorporation by β-cells (FIG. 16c). The results were confirmed in a separate cohort of animals in which β-cells were identified by the presence of insulin rather than PDX-1 (FIG. 20). Notably, treatment with ABT-702 did not increase the replication rate of exocrine cells, again highlighting the selectivity of ADK-Is (FIGS. 16d and 20). In addition, BRDU incorporation by hepatocytes in response to ABT-702 treatment in the same cohorts of animals was also examined (FIG. 16e). Hepatocytes did not show an increased rate of cell division in response to drug treatment. Therefore, ABT-702 selectively promoted β-cell replication in vitro and in vivo.

### Discussion

DM2 is a global epidemic in need of improved treatment. While disease prevention through modification of dietary and behavioral practices remains paramount, these strategies have not been broadly successful. Historically, DM2 therapies have attempted to augment insulin secretion (sulfonylureas) or reduce insulin demand (biguanides, thiazolidinediones) by lowering peripheral resistance. However, DM2 patients appear to have a limited capacity for adaptive β-cell growth and these approaches do not address this deficiency. Consequently, most diabetic patients demonstrate progressive β-cell failure. Accordingly, the invention provides a platform to identify pharmacologic agents that promote increased β-cell division. While the present study focused upon a single class of agents, adenosine kinase inhibitors, the screening assay described herein can be used to discover additional compounds.

The application of growth promoting agents to enhance β-cell mass is primarily limited by their specificity. A broad spectrum of factors that promote β-cell replication have been identified including cell cycle regulators such as cyclin2, signaling molecules like AKT2, growth factors and hormones including hepatocyte growth factor, growth hormone and prolactin and several small molecules (S. Georgia & A. Bhushan, The Journal of clinical investigation 114, 963-968 (2004); S. Fatrai, et al., Diabetes 55, 318-325 (2006); J. H. Nielsen, et al., Diabetes 50 Suppl 1, S25-29 (2001); R. C. Vasavada, et al., The international journal of biochemistry & cell biology 38, 931-950 (2006); and W. Wang, et al., Proceedings of the National Academy of Sciences of the United States of America 106, 1427-1432 (2009)). However, most of these entities demonstrate pleiotropic effects that limit their utility or have failed to consistently increase β-cell replication. The invention provides ADK-Is as a new class of agents which are capable of promoting β-cell replication in vitro and in vivo. Of critical importance is that these compounds have a selective proliferation effect on β-cells and not α-cells, hepatocytes, exocrine cells or fibroblasts. Additionally, results presented herein show that ADK-Is have the attractive quality of augmenting the β-cell replication effect of GLP-1R agonists and hyperglycemia.

The identification of ADK as a regulator of β-cell replication was an unexpected finding which highlights the value of using chemical screening to reveal new biology. In nearly all other cell types that have been treated with adenosine or an ADK-Is, growth inhibition is observed. In cardiac fibroblasts, aortic smooth muscle cells, glomerular mesangial cells, chondrocytes, astrocytes, lymphocytes, colon cancer cell lines, breast cancer cell lines, gastric cancer cell lines, hepatoma cell lines and thyroid cell lines, to name a few, the effect of ADK-Is is to inhibit, not stimulate, replication. See for example, R. K. Dubey, et al., Circulation 96, 2656-2666 (1997); R. K. Dubey, et al., Hypertension 31, 516-521 (1998); R. K. Dubey, et al., Hypertension 46, 628-634 (2005); Mistry, M. G. Chambers & R. M. Mason, Osteoarthritis and cartilage/OARS, Osteoarthritis Research Society 14, 486-495 (2006); J. Faller, et al., Metabolism: clinical and experimental 33, 369-374 (1984); M. Saito, et al., Cancer letters 290, 211-215; Y. Yasuda, et al., Journal of gastroenterology 44, 56-65 (2009); M. Hashemi, et al., Cell proliferation 38, 269-285 (2005); K. Sai, et al., Neurotoxicology 27, 458-467 (2006); M. Saitoh, et al., Biochemical pharmacology 67, 2005-2011 (2004); L. T. Wen & A. F. Knowles, British journal of pharmacology 140, 1009-1018 (2003); L. F. Wu, et al., Acta pharmacologica Sinica 27, 477-484 (2006); and M. Vainio, P. Saarinen & K. Tornquist, Journal of cellular physiology 171, 336-342 (1997). These prior results are consistent with our observations that hepatocytes, fibroblasts, exocrine cells and α-cells do not proliferate in response ADK-Is.

Without wishing to be bound by a theory, the unusual growth promoting effect of ADK-Is on PDX-1⁺ cells is unlikely to be mediated by extracellular adenosine signaling for three reasons. First, a cell autonomous increase in β-cell proliferation in response to ADK knock-down was observed. If this effect were mediated by extracellular adenosine, a paracrine effect would be anticipated. Second, the addition of adenosine or adenosine receptor antagonists had no effect on β-cell proliferation in the assay (data not shown). Third, β-cells express primarily the nuclear isoform of ADK is thought to primarily regulate transmethylation activity. Without wishing to be bound by a theory, inhibition of ADK activity prevents methylation reactions that act to prevent β-cell replication. For example, menin is an important negative regulator of β-cell replication that functions as part of a histone methyltransferase complex. (S. K. Karnik, et al., Proceedings of the National Academy of Sciences of the United States of America 102, 14659-14664 (2005)). Accordingly, menin activity can be inhibited by ADK-inhibition.

ADK-Is appear to be well tolerated by animals and are in development as a therapeutic for a variety of conditions including epilepsy, cerebral ischemia, pain and inflammation. (E. A. Kowaluk & M. F. Jarvis, Expert opinion on investigational drugs 9, 551-564 (2000)) The invention provides a previously unrecognized regulator of β-cell replication with a selective activity on β-cell replication. Thus, ADK can be a therapeutic target for the treatment and prevention of diabetes. Furthermore, ex vivo expansion of β-cells allows overcoming the limited availability of cadaveric islets used for treating type 1 diabetes.

### REFERENCES

1. R. C. Turner, C. A. Cull, V. Frighi, R. R. Holman, Glycemic control with diet, sulfonylurea, metformin, or insulin in patients with type 2 diabetes mellitus: progressive requirement for multiple therapies (UKPDS 49). UK Prospective Diabetes Study (UKPDS) Group. Jama 281, 2005-2012 (1999).
2. S. M. Haffner, S. Lehto, T. Ronnemaa, K. Pyorala, M. Laakso, Mortality from coronary heart disease in subjects with type 2 diabetes and in nondiabetic subjects with and without prior myocardial infarction. The New England journal of medicine 339, 229-234 (1998).
3. Juutilainen, S. Lehto, T. Ronnemaa, K. Pyorala, M. Laakso, Type 2 diabetes as a “coronary heart disease equivalent”: an 18-year prospective population-based study in Finnish subjects. Diabetes care 28, 2901-2907 (2005).
4. E. S. Huang, A. Basu, M. O'Grady, J. C. Capretta, Projecting the future diabetes population size and related costs for the U.S. Diabetes care 32, 2225-2229 (2009).
5. J. P. Boyle, T. J. Thompson, E. W. Gregg, L. E. Barker, D. F. Williamson, Projection of the year 2050 burden of diabetes in the US adult population: dynamic modeling of incidence, mortality, and prediabetes prevalence. Population health metrics 8, 29.
6. D. M. Muoio, C. B. Newgard, Mechanisms of disease: molecular and metabolic mechanisms of insulin resistance and beta-cell failure in type 2 diabetes. Nat Rev Mol Cell Biol 9, 193-205 (2008).
7. B. L. Wajchenberg, beta-cell failure in diabetes and preservation by clinical treatment. Endocrine reviews 28, 187-218 (2007).
8. S. O'Rahilly, Human genetics illuminates the paths to metabolic disease. Nature 462, 307-314 (2009).
9. S. C. Elbein, S. J. Hasstedt, K. Wegner, S. E. Kahn, Heritability of pancreatic beta-cell function among nondiabetic members of Caucasian familial type 2 diabetic kindreds. The Journal of clinical endocrinology and metabolism 84, 1398-1403 (1999).
10. E. Butler, J. Janson, S. Bonner-Weir, R. Ritzel, R. A. Rizza, P. C. Butler, Beta-cell deficit and increased beta-cell apoptosis in humans with type 2 diabetes. Diabetes 52, 102-110 (2003).
11. H. Mokdad, E. S. Ford, B. A. Bowman, W. H. Dietz, F. Vinicor, V. S. Bales, J. S. Marks, Prevalence of obesity, diabetes, and obesity-related health risk factors, 2001. Jama 289, 76-79 (2003).
12. R. N. Kulkarni, U.S. Jhala, J. N. Winnay, S. Krajewski, M. Montminy, C. R. Kahn, PDX-1 haploinsufficiency limits the compensatory islet hyperplasia that occurs in response to insulin resistance. The Journal of clinical investigation 114, 828-836 (2004).
13. R. A. Ritzel, A. E. Butler, R. A. Rizza, J. D. Veldhuis, P. C. Butler, Relationship between beta-cell mass and fasting blood glucose concentration in humans. Diabetes care 29, 717-718 (2006).
14. Y. Dor, J. Brown, O. I. Martinez, D. A. Melton, Adult pancreatic beta-cells are formed by self-duplication rather than stem-cell differentiation. Nature 429, 41-46 (2004).
15. K. Brennand, D. Huangfu, D. Melton, All beta Cells Contribute Equally to Islet Growth and Maintenance. PLoS biology 5, e163 (2007).
16. S. Georgia, A. Bhushan, Beta cell replication is the primary mechanism for maintaining postnatal beta cell mass. The Journal of clinical investigation 114, 963-968 (2004).
17. M. Teta, M. M. Rankin, S. Y. Long, G. M. Stein, J. A. Kushner, Growth and regeneration of adult beta cells does not involve specialized progenitors. Developmental cell 12, 817-826 (2007).
18. T. Nir, D. A. Melton, Y. Dor, Recovery from diabetes in mice by beta cell regeneration. The Journal of clinical investigation 117, 2553-2561 (2007).
19. D. A. Cano, I. C. Rulifson, P. W. Heiser, L. B. Swigart, S. Pelengaris, M. German, G. I. Evan, J. A. Bluestone, M. Hebrok, Regulated-cell regeneration in the adult mouse pancreas. Diabetes (2007).
20. P. Collombat, X. Xu, P. Ravassard, B. Sosa-Pineda, S. Dussaud, N. Billestrup, O. D. Madsen, P. Serup, H. Heimberg, A. Mansouri, The ectopic expression of Pax4 in the mouse pancreas converts progenitor cells into alpha and subsequently beta cells. Cell 138, 449-462 (2009).
21. Inada, C. Nienaber, H. Katsuta, Y. Fujitani, J. Levine, R. Morita, A. Sharma, S. Bonner-Weir, Carbonic anhydrase II-positive pancreatic cells are progenitors for both endocrine and exocrine pancreas after birth. Proceedings of the National Academy of Sciences of the United States of America 105, 19915-19919 (2008).
22. X. Xu, J. D'Hoker, G. Stange, S. Bonne, N. De Leu, X. Xiao, M. Van de Casteele, G. Mellitzer, Z. Ling, D. Pipeleers, L. Bouwens, R. Scharfmann, G. Gradwohl, H. Heimberg, Beta cells can be generated from endogenous progenitors in injured adult mouse pancreas. Cell 132, 197-207 (2008).
23. F. Thorel, V. Nepote, I. Avril, K. Kohno, R. Desgraz, S. Chera, P. L. Herrera, Conversion of adult pancreatic alpha-cells to beta-cells after extreme beta-cell loss. Nature 464, 1149-1154 (2010).
24. F. A. Van Assche, L. Aerts, F. De Prins, A morphological study of the endocrine pancreas in human pregnancy. British journal of obstetrics and gynaecology 85, 818-820 (1978).
25. G. Xu, D. A. Stoffers, J. F. Habener, S. Bonner-Weir, Exendin-4 stimulates both beta-cell replication and neogenesis, resulting in increased beta-cell mass and improved glucose tolerance in diabetic rats. Diabetes 48, 2270-2276 (1999).
26. Y. Li, T. Hansotia, B. Yusta, F. R is, P. A. Halban, D. J. Drucker, Glucagon-like peptide-1 receptor signaling modulates beta cell apoptosis. The Journal of biological chemistry 278, 471-478 (2003).
27. D. J. Drucker, M. A. Nauck, The incretin system: glucagon-like peptide-1 receptor agonists and dipeptidyl peptidase-4 inhibitors in type 2 diabetes. Lancet 368, 1696-1705 (2006).
28. W. Wang, J. R. Walker, X. Wang, M. S. Tremblay, J. W. Lee, X. Wu, P. G. Schultz, Identification of small-molecule inducers of pancreatic beta-cell expansion. Proceedings of the National Academy of Sciences of the United States of America 106, 1427-1432 (2009).
29. N. Fleischer, C. Chen, M. Surana, M. Leiser, L. Rossetti, W. Pralong, S. Efrat, Functional analysis of a conditionally transformed pancreatic beta-cell line. Diabetes 47, 1419-1425 (1998).
30. Y. Zimmer, D. Milo-Landesman, A. Svetlanov, S. Efrat, Genes induced by growth arrest in a pancreatic beta cell line: identification by analysis of cDNA arrays. FEBS letters 457, 65-70 (1999).
31. P. Serup, H. V. Petersen, E. E. Pedersen, H. Edlund, J. Leonard, J. S. Petersen, L. I. Larsson, O. D. Madsen, The homeodomain protein IPF-1/STF-1 is expressed in a subset of islet cells and promotes rat insulin 1 gene expression dependent on an intact E1 helix-loop-helix factor binding site. The Biochemical journal 310 (Pt 3), 997-1003 (1995).
32. J. Suckale, M. Solimena, Pancreas islets in metabolic signaling—focus on the beta-cell. Front Biosci 13, 7156-7171 (2008).
33. E. A. Kowaluk, M. F. Jarvis, Therapeutic potential of adenosine kinase inhibitors. Expert opinion on investigational drugs 9, 551-564 (2000).
34. P. Bork, C. Sander, A. Valencia, Convergent evolution of similar enzymatic function on different protein folds: the hexokinase, ribokinase, and galactokinase families of sugar kinases. Protein Sci 2, 31-40 (1993).
35. T. A. Krenitsky, R. L. Miller, J. A. Fyfe, Levels of nucleoside and nucleotide kinases in rhesus monkey tissues. Biochemical pharmacology 23, 70-72 (1974).
36. J. Park, R. S. Gupta, Adenosine kinase and ribokinase—the RK family of proteins. Cell Mol Life Sci 65, 2875-2896 (2008).
37. M. Andres, I. H. Fox, Purification and properties of human placental adenosine kinase. The Journal of biological chemistry 254, 11388-11393 (1979).
38. X. A. Cui, B. Singh, J. Park, R. S. Gupta, Subcellular localization of adenosine kinase in mammalian cells: The long isoform of AdK is localized in the nucleus. Biochemical and biophysical research communications 388, 46-50 (2009).
39. N. M. Kredich, D. V. Martin, Jr., Role of S-adenosylhomocysteine in adenosinemediated toxicity in cultured mouse T lymphoma cells. Cell 12, 931-938 (1977).
40. Boison, L. Scheurer, V. Zumsteg, T. Rulicke, P. Litynski, B. Fowler, S. Brandner, H. Mohler, Neonatal hepatic steatosis by disruption of the adenosine kinase gene. Proceedings of the National Academy of Sciences of the United States of America 99, 6985-6990 (2002).
41. M. Braun, R. Ramracheya, S. Amisten, M. Bengtsson, Y. Moritoh, Q. Zhang, P. R. Johnson, P. Rorsman, Somatostatin release, electrical activity, membrane currents and exocytosis in human pancreatic delta cells. Diabetologia 52, 1566-1578 (2009).
42. S. Bonner-Weir, D. Deery, J. L. Leahy, G. C. Weir, Compensatory growth of pancreatic beta-cells in adult rats after short-term glucose infusion. Diabetes 38, 49-53 (1989).
43. L. C. Alonso, T. Yokoe, P. Zhang, D. K. Scott, S. K. Kim, C. P. O'Donnell, A. Garcia-Ocana, Glucose infusion in mice: a new model to induce beta-cell replication. Diabetes 56, 1792-1801 (2007).
44. J. H. Nielsen, E. D. Galsgaard, A. Moldrup, B. N. Friedrichsen, N. Billestrup, J. A. Hansen, Y. C. Lee, C. Carlsson, Regulation of beta-cell mass by hormones and growth factors. Diabetes 50 Suppl 1, S25-29 (2001).
45. G. Z. Zheng, C. Lee, J. K. Pratt, R. J. Perner, M. Q. Jiang, A. Gomtsyan, M. A. Matulenko, Y. Mao, J. R. Koenig, K. H. Kim, S. Muchmore, H. Yu, K. Kohlhaas, K. M. Alexander, S. McGaraughty, K. L. Chu, C. T. Wismer, J. Mikusa, M. F. Jarvis, K. Marsh, E. A. Kowaluk, S. S. Bhagwat, A. O. Stewart, Pyridopyrimidine analogues as novel adenosine kinase inhibitors. Bioorganic & medicinal chemistry letters 11, 2071-2074 (2001).
46. S. Fatrai, L. Elghazi, N. Balcazar, C. Cras-Meneur, I. Krits, H. Kiyokawa, E. Bernal-Mizrachi, Akt induces beta-cell proliferation by regulating cyclin D1, cyclin D2, and p21 levels and cyclin-dependent kinase-4 activity. Diabetes 55, 318-325 (2006).
47. R. C. Vasavada, J. A. Gonzalez-Pertusa, Y. Fujinaka, N. Fiaschi-Taesch, I. Cozar-Castellano, A. Garcia-Ocana, Growth factors and beta cell replication. The international journal of biochemistry & cell biology 38, 931-950 (2006).
48. R. K. Dubey, D. G. Gillespie, Z. Mi, E. K. Jackson, Exogenous and endogenous adenosine inhibits fetal calf serum-induced growth of rat cardiac fibroblasts: role of A2B receptors. Circulation 96, 2656-2666 (1997).
49. R. K. Dubey, D. G. Gillespie, Z. Mi, E. K. Jackson, Adenosine inhibits growth of human aortic smooth muscle cells via A2B receptors. Hypertension 31, 516-521 (1998).
50. R. K. Dubey, D. G. Gillespie, Z. Mi, E. K. Jackson, Adenosine inhibits PDGF-induced growth of human glomerular mesangial cells via A(2B) receptors. Hypertension 46, 628-634 (2005).
51. Mistry, M. G. Chambers, R. M. Mason, The role of adenosine in chondrocyte death in murine osteoarthritis and in a murine chondrocyte cell line. Osteoarthritis and cartilage/OARS, Osteoarthritis Research Society 14, 486-495 (2006).
52. J. Faller, T. D. Palella, P. Dean, I. H. Fox, Altered cell cycle distributions of cultured human lymphoblasts during cytotoxicity related to adenosine deaminase inhibition. Metabolism: clinical and experimental 33, 369-374 (1984).
53. M. Saito, T. Yaguchi, Y. Yasuda, T. Nakano, T. Nishizaki, Adenosine suppresses CW2 human colonic cancer growth by inducing apoptosis via A(1) adenosine receptors. Cancer letters 290, 211-215.
54. Y. Yasuda, M. Saito, T. Yamamura, T. Yaguchi, T. Nishizaki, Extracellular adenosine induces apoptosis in Caco-2 human colonic cancer cells by activating caspase-9/-3 via A(2a) adenosine receptors. Journal of gastroenterology 44, 56-65 (2009).
55. M. Hashemi, F. Karami-Tehrani, S. Ghavami, S. Maddika, M. Los, Adenosine and deoxyadenosine induces apoptosis in oestrogen receptor-positive and -negative human breast cancer cells via the intrinsic pathway. Cell proliferation 38, 269-285 (2005).
56. K. Sai, D. Yang, H. Yamamoto, H. Fujikawa, S. Yamamoto, T. Nagata, M. Saito, T. Yamamura, T. Nishizaki, A(1) adenosine receptor signal and AMPK involving caspase-9/-3 activation are responsible for adenosine-induced RCR-1 astrocytoma cell death. Neurotoxicology 27, 458-467 (2006).
57. M. Saitoh, K. Nagai, K. Nakagawa, T. Yamamura, S. Yamamoto, T. Nishizaki, Adenosine induces apoptosis in the human gastric cancer cells via an intrinsic pathway relevant to activation of AMP-activated protein kinase. Biochemical pharmacology 67, 2005-2011 (2004).
58. L. T. Wen, A. F. Knowles, Extracellular ATP and adenosine induce cell apoptosis of human hepatoma Li-7A cells via the A3 adenosine receptor. British journal of pharmacology 140, 1009-1018 (2003).
59. L. F. Wu, G. P. Li, J. L. Feng, Z. J. Pu, Molecular mechanisms of adenosine-induced apoptosis in human HepG2 cells. Acta pharmacologica Sinica 27, 477-484 (2006).
60. M. Vainio, P. Saarinen, K. Tornquist, Adenosine inhibits DNA synthesis stimulated with TSH, insulin, and phorbol 12-myristate 13-acetate in rat thyroid FRTL-5 cells. Journal of cellular physiology 171, 336-342 (1997).
61. S. K. Karnik, C M. Hughes, X. Gu, O. Rozenblatt-Rosen, G. W. McLean, Y. Xiong, M. Meyerson, S. K. Kim, Menin regulates pancreatic islet growth by promoting histone methylation and expression of genes encoding p27Kip1 and p18INK4c. Proceedings of the National Academy of Sciences of the United States of America 102, 14659-14664 (2005).
62. M. Gotoh, T. Maki, S. Satomi, J. Porter, S. Bonner-Weir, C. J. O'Hara, A. P. Monaco, Reproducible high yield of rat islets by stationary in vitro digestion following pancreatic ductal or portal venous collagenase injection. Transplantation 43, 725-730 (1987).
63. P. O. Seglen, Preparation of isolated rat liver cells. Methods in cell biology 13, 29-83 (1976).

All patents and other publications identified in the specification and examples are expressly incorporated herein by reference for all purposes. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1. A method of increasing β-cell replication in a population of pancreatic cells, the method comprising: contacting a population of pancreatic cells with an inhibitor of adenosine kinase (ADK), an inhibitor of S-Adenosylhomocysteine hydrolase (SAHH), or an activator of AMP activated protein kinase (AMPK).

2. The method of claim 1, wherein the inhibitor of adenosine kinase is of formula (I): 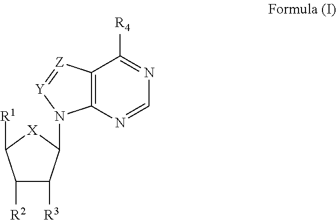
wherein:
R¹ is H, optionally substituted C₁-C₆ alkyl, optionally substituted C₂-C₆ alkenyl, optionally substituted C₂-C₆ alkynyl, OR⁵, SR⁵, N(R⁶)₂, (CH₂)ₘR⁷, or R¹ and R² together with the atoms they are attached to form 5-8 membered heterocycle which can be optionally substituted;
R² and R³ are each independently H, OR⁵, SR⁵, N(R⁵)₂, or R² and R³ together with the atoms they are attached to form 5-8 membered heterocyclyl which can be optionally substituted;
R⁴ is H, halogen, CN, N₂, OR⁵, SR⁵, N(R⁵)₂, optionally substituted C₁-C₆ alkyl, optionally substituted C₂-C₆ alkenyl, optionally substituted C₂-C₆ alkynyl, optionally substituted aryl, optionally substituted heteroaryl, optionally substituted cyclyl, optionally substituted heterocyclyl, optionally substituted arylalkyl, or optionally substituted heteroarylalkyl;
R⁵ is independently for each occurrence H, C(O)R⁷, C(O)OR⁷, C(O)N(R⁷)₂, optionally substituted C₁-C₆ alkyl, optionally substituted C₂-C₆ alkenyl, optionally substituted C₂-C₆ alkynyl, optionally substituted aryl, optionally substituted heteroaryl, optionally substituted cyclyl, optionally substituted heterocyclyl, optionally substituted arylalkyl, optionally substituted heteroarylalkyl, or the two R⁵ taken together with the nitrogen atom to which they are attached form a 5-to-7 membered ring optionally comprising 1-3-additional heteroatoms selected from N, O or S;
R⁶ is R⁵, OR⁵, SR⁵, N(R⁵)₂, N₂, CN, halogen, or 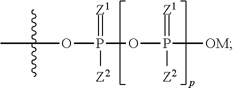
R⁷ is independently for each occurrence H, optionally substituted C₁-C₆ alkyl, optionally substituted C₂-C₆ alkenyl, optionally substituted C₂-C₆ alkynyl, optionally substituted aryl, optionally substituted heteroaryl, optionally substituted cyclyl, optionally substituted heterocyclyl, optionally substituted arylalkyl, or optionally substituted heteroarylalkyl;
X is O, S, NH, or CH₂;
Y and Z are each independently N or CR⁸;
R⁸ is independently for each occurrence H, halogen, CN, C(O)R⁷, C(O)OR⁷, C(O)N(R⁷)₂, optionally substituted C₁-C₆ alkyl, optionally substituted C₂-C₆ alkenyl, optionally substituted C₂-C₆ alkynyl, optionally substituted aryl, optionally substituted heteroaryl, optionally substituted cyclyl, optionally substituted heterocyclyl, optionally substituted arylalkyl, or optionally substituted heteroarylalkyl;
Z¹ is independently for each occurrence O or S;
Z² is independently for each occurrence OM, SM, OR⁵, SR⁵, N(R⁵)₂, optionally substituted C₁-C₆ alkyl, optionally substituted C₂-C₆ alkenyl, optionally substituted C₂-C₆ alkynyl, optionally substituted aryl, optionally substituted heteroaryl, optionally substituted cyclyl, optionally substituted heterocyclyl, optionally substituted arylalkyl, or optionally substituted heteroarylalkyl;
M is an alkali metal cation;
m is 1, 2, 3, or 4;
n is 0, 1, or 2; and
pharmaceutically acceptable salts and amides thereof.

3. The method of claim 1, wherein the inhibitor of adenosine kinase is of formula (II): 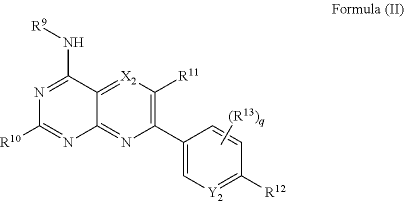
wherein:
each R⁹ is independently H, optionally substituted C₁-C₆ alkyl, optionally substituted C₂-C₆ alkenyl, optionally substituted C₂-C₆ alkynyl, optionally substituted aryl, optionally substituted heteroaryl, optionally substituted cyclyl, optionally substituted heterocyclyl, optionally substituted arylalkyl, optionally substituted heteroarylalkyl, or the two R⁹ taken together with the nitrogen atom to which they are attached form a 5-to-7 membered ring which optionally comprises 1-3-additional heteroatoms selected from N, O or S;
R¹⁰, R¹¹ and R¹² are each independently H, OR¹⁴, N(R¹⁴)₂, N₂, NO₂, CN, halogen, optionally substituted C₁-C₆ alkyl, optionally substituted C₂-C₆ alkenyl, optionally substituted C₂-C₆ alkynyl, optionally substituted aryl, optionally substituted heteroaryl, optionally substituted cyclyl, optionally substituted heterocyclyl, optionally substituted arylalkyl, or optionally substituted heteroarylalkyl;
R¹³ is independently for each occurrence halogen, CN, NH₂, or optionally substituted C₁-C₆ alkyl;
R¹⁴ is H, optionally substituted C₁-C₆ alkyl, optionally substituted C₂-C₆ alkenyl, optionally substituted C₂-C₆ alkynyl, optionally substituted aryl, optionally substituted heteroaryl, optionally substituted cyclyl, optionally substituted heterocyclyl, optionally substituted arylalkyl, optionally substituted heteroarylalkyl, or the two R¹⁴ taken together with the nitrogen atom to which they are attached form a 5-to-7 membered ring which optionally comprises 1-3-additional heteroatoms selected from N, O or S;
X₂ is N or CR¹⁵;
R¹⁵ is NHR¹⁶, optionally substituted C₁-C₆ alkyl, optionally substituted C₂-C₆ alkenyl, optionally substituted C₂-C₆ alkynyl, optionally substituted aryl, optionally substituted heteroaryl, optionally substituted cyclyl, optionally substituted heterocyclyl, optionally substituted arylalkyl, or optionally substituted heteroarylalkyl;
R¹⁶ is H, optionally substituted C₁-C₆ alkyl, optionally substituted C₂-C₆ alkenyl, optionally substituted C₂-C₆ alkynyl, optionally substituted aryl, optionally substituted heteroaryl, optionally substituted cyclyl, optionally substituted heterocyclyl, optionally substituted arylalkyl, or optionally substituted heteroarylalkyl;
Y₂ is N or CH;
q is 0, 1, 2, or 3; and
pharmaceutically acceptable salts and amides thereof.

4. (canceled)

5. (canceled)

6. The method of claim 1, wherein the ADK inhibitor is selected from the group consisting of aristeromycin, 5′-deoxyadenosine, 5′-aminoadenosine, 5′-deoxy-5-iodotubercidin, 5-iodotubercidin (A10), 7-deaza-7-iodo-2′,3′-dideoxyadenosine, nor-aristeromycin, nor-tubercidin, A-134974, Toyocamycin, GP-515 ((2R,3R,4S,5R)-2-(4-amino-3-bromo-1H-pyrazolo[3,4-d]pyrimidin-1-yl)-5-(aminomethyl)-tetrahydrofuran-3,4-diol), GP-3269 ((2R,3R,4S,5R)-2-(4-(4-fluorophenylamino)-5-phenyl-7H-pyrrolo[2,3-d]pyrimidin-7-yl)-tetrahydro-5-methylfuran-3,4-diol), GP-683 ((2R,3S,4R,5R)-tetrahydro-2-methyl-5-(5-phenyl-4-(phenylamino)-7H-pyrrolo[2,3-d]pyrimidin-7-yl)furan-3,4-diol), GP-947 ((2S,3S,4R,5R)-tetrahydro-2-methyl-5-(5-phenyl-4-(phenylamino)-7H-pyrrolo[2,3-d]pyrimidin-7-yl)furan-3,4-diol), ABT-702 (5-(3-bromophenyl)-7-(6-morpholinopyridin-3-yl)pyrido[2,3-d]pyrimidin-4-amine, B8), compound 1 ((2R,3R,4S,5R)-2-(4-amino-5-iodo-7H-pyrrolo[2,3-d]pyrimidin-7-yl)-5-(aminomethyl)-tetrahydrofuran-3,4-diol), compound 2 ((2R,3R,4S,5R)-2-(4-chloro-5-iodo-7H-pyrrolo[2,3-d]pyrimidin-7-yl)-5-(aminomethyl)-tetrahydrofuran-3,4-diol), compound 3 ((1S,2R,3S,5R)-3-amino-5-(6-amino-9H-purin-9-yl)cyclopentane-1,2-diol), compound 4 ((1S,2R,3S,5R)-3-amino-5-(7-amino-3H-[1,2,3]triazolo[4,5-d]pyrimidin-3-yl)cyclopentane-1,2-diol), compound 5 ((1S,2R,3S,4R)-4-(4-amino-5-iodo-7H-pyrrolo[2,3-d]pyrimidin-7-yl)cyclopentane-1,2,3-triol), compound 6 (7-(4-(dimethylamino)phenyl)pteridin-4-amine), compound 7 (5-(3-bromophenyl)-7-(4-(dimethylamino)phenyl)pyrido[2,3-d]pyrimidin-4-amine), compound 8 (5-(2-bromobenzyl)-7-(6-morpholinopyridin-3-yl)pyrido[2,3-d]pyrimidin-4-amine), compound 9 (5-cyclohexyl-7-(6-morpholinopyridin-3-yl)pyrido[2,3-d]pyrimidin-4-amine), compound 10 (5-(tetrahydro-2H-pyran-4-yl)-7-(6-morpholinopyridin-3-yl)pyrido[2,3-d]pyrimidin-4-amine), compound 11 (5-(1-(2-bromophenyl)ethyl)-7-(6-morpholinopyridin-3-yl)pyrido[2,3-d]pyrimidin-4-amine), compound 12 (5-(2-methylpent-4-en-2-yl)-7-(6-morpholinopyridin-3-yl)pyrido[2,3-d]pyrimidin-4-amine), compound 13 (N5-((1H-indol-3-yl)methyl)-7-(6-morpholinopyridin-3-yl)pyrido[2,3-d]pyrimidine-4,5-diamine), and combinations thereof.

7. The method of claim 1, wherein the activator of AMPK is of formula (III): 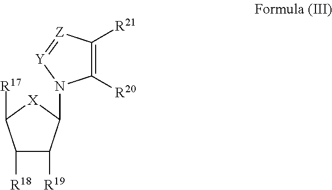
wherein:
R¹⁷ is H, optionally substituted C₁-C₆ alkyl, optionally substituted C₂-C₆ alkenyl, optionally substituted C₂-C₆ alkynyl, OR²², SR²², N(R²²)₂, (CH₂)ₘR²³, or R¹⁷ and R¹⁸ together with the atoms they are attached to form 5-8 membered heterocycle which can be optionally substituted;
R¹⁸ and R¹⁹ are each independently H, OR²², SR²², N(R²²)₂, O or R¹⁸ and R¹⁹ together with the atoms they are attached to form 5-8 membered heterocycle which can be optionally substituted;
R²⁰ and R²¹ are each independently halogen, CN, N₂, OR²², SR²², N(R²²)₂, C(O)R²⁴, C(O)OR²⁴, C(O)N(R²⁴)₂, optionally substituted C₁-C₆ alkyl, optionally substituted C₂-C₆ alkenyl, optionally substituted C₂-C₆ alkynyl, optionally substituted aryl, optionally substituted heteroaryl, optionally substituted cyclyl, optionally substituted heterocyclyl, optionally substituted arylalkyl, or optionally substituted heteroarylalkyl;
R²² is independently for each occurrence H, C(O)R²⁴, C(O)OR²⁴, C(O)N(R²⁴)₂, optionally substituted C₁-C₆ alkyl, optionally substituted C₂-C₆ alkenyl, optionally substituted C₂-C₆ alkynyl, optionally substituted aryl, optionally substituted heteroaryl, optionally substituted cyclyl, optionally substituted heterocyclyl, optionally substituted arylalkyl, or optionally substituted heteroarylalkyl;
R²³ is R²², OR²², SR²², N(R²²)₂, N₂, CN, halogen, or 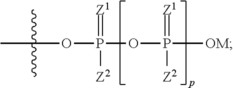
R²⁴ is independently for each occurrence H, optionally substituted C₁-C₆ alkyl, optionally substituted C₂-C₆ alkenyl, optionally substituted C₂-C₆ alkynyl, optionally substituted aryl, optionally substituted heteroaryl, optionally substituted cyclyl, optionally substituted heterocyclyl, optionally substituted arylalkyl, or optionally substituted heteroarylalkyl;
X is O, S, NH, or CH₂;
Y and Z are each independently N or CR²⁵;
R²⁵ is independently for each occurrence H, halogen, CN, C(O)R²⁴, C(O)OR²⁴, C(O)N(R²⁴)₂, optionally substituted C₁-C₆ alkyl, optionally substituted C₂-C₆ alkenyl, optionally substituted C₂-C₆ alkynyl, optionally substituted aryl, optionally substituted heteroaryl, optionally substituted cyclyl, optionally substituted heterocyclyl, optionally substituted arylalkyl, or optionally substituted heteroarylalkyl;
Z¹ is independently for each occurrence O or S;
Z² is independently for each occurrence H, OM, SM, OR²², SR²², N(R²²)₂, optionally substituted C₁-C₆ alkyl, optionally substituted C₂-C₆ alkenyl, optionally substituted C₂-C₆ alkynyl, optionally substituted aryl, optionally substituted heteroaryl, optionally substituted cyclyl, optionally substituted heterocyclyl, optionally substituted arylalkyl, or optionally substituted heteroarylalkyl;
M is an alkali metal cation;
m is 1, 2, 3, or 4;
n is 0, 1, or 2; and
pharmaceutically acceptable salts and amides thereof.

8. (canceled)

9. (canceled)

10. The method of claim 1, wherein the AMPK activator is 5-aminoimidazole-4-carboxamide ribonucleoside (AICAR).

11. (canceled)

12. (canceled)

13. The method of claim 1, wherein the SAHH inhibitor is selected from the group consisting of 9(S)-(2,3-dihydroxypropyl)adenine [(S)-DHPA]; D-eritadine; (R,S)-3-adenine-9-yl-2-hydroxypropanoic acid [(R,S)-AHPA]; adenosine (Ado) dialdehyde; 3-deazaadenosine (c3-Ado); aristeromycin (Ari); neplanocin A (NPA or NpcA); dihydroxycyclopentenyladenine (DHCeA); dihydroxycyclopentenyl-3-deazaadenine (c3-DHCeA); dihydroxycyclopentanyladenine (DHCaA); dihydroxycyclopentanyl-3-deazaadenine (c3-DHCaA); 3-deazaneplanocin A (c3-NpcA); 3-deazaaristeromycin (c3Ari); carbocyclic-3-deazaadenosine (C-c3Ado); 6′-Cmethylneplanocin A; 2′-deoxyadenosine; tubercidin; ribavirin; pyraazofurin; 2′-deoxy-2′-chloroadenosine; isopentenyladenosine; methylthioadenosine (MTA); 9-β-arabinofuranosyladenine (Ara-A, vidarabine); 2′-Deoxyadenosine; N-methylaristeromycin, 8-azaaristeromycin and 3-deazaaristeromycin, and their dialdehyde and diol derivatives; (±)-5Noraristeromycin and its 2,6-diamino-analogue; 2′-deoxy-aristeromycin; 3′-deoxy-aristeromycin; 3′-amino-3′-deoxy-aristeromycin; 3′-amino-3′-deoxyarabinofuranosyl-aristeromycin; 6′-hydroxy-aristeromycin; 6′-mercapto-aristeromycin; 8′-bromo-aristeromycin; 8-hydroxyaristeromycin, aristeromycin-3′-cyclic phosphate, aristeromycin-6′-cyclic phosphate; 2-fluoro-5-adenosylhomocysteine (2-FSAH); S-Adenosyl-L-homocysteine sulfoxide; S-Adenosyl-Lhomocysteine sulfone; S-aristeromycinyl-L-homocysteine; 5′-S-(3-carboxyl-4-nitrophenyl)thioadenosine; 5′-S(methyl)-5′-S-(butyl)thioadenosine; and any combinations thereof.

14. The method of claim 1, wherein the pancreatic cells are from a subject.

15. (canceled)

16. (canceled)

17. (canceled)

18. (canceled)

19. The method of claim 1, wherein the pancreatic cells are primary pancreatic cells.

20. The method of claim 1, wherein the pancreatic cells are derived from de-differentiated cells.

21. The method of claim 1, wherein the contact is in vitro, ex vivo, or in vivo.

22. (canceled)

23. (canceled)

24. (canceled)

25. (canceled)

26. (canceled)

27. (canceled)

28. The method of claim 14, wherein the subject suffers from Type 1 or Type II diabetes.

29. (canceled)

30. (canceled)

31. (canceled)

32. (canceled)

33. A high throughput assay for screening compounds that increases β-cell replication in a population of pancreatic cells, the assay comprising:
(a) contacting a population or preparation of pancreatic cells with a test compound, wherein the pancreatic cells are primary pancreatic cells;
(b) assessing beta-cell replication or growth; and
(c) selecting the compound that increases or enhances β-cell replication or growth.

34. The assay of claim 33, wherein the pancreatic cells are cultured in a cell culture vessel, surface of which is coated with conditioned media from rat bladder carcinoma cell line 804G.

35. The assay of claim 33, wherein the step of assessing beta-cell replication comprises detecting a beta-cell marker and a cell-replication marker.

36. The assay of claim 35, wherein the cell-replication marker is Ki-67 or PH3.

37. The assay of claim 33, wherein the β-cell marker is selected from the group consisting of PDX-1, insulin, c-peptide, amylin, E-cadherin, Hnf3β, PCI/3, Beta2, Nkx2.2, Nkx6.1, GLUT2, PC2, ZnT-8, MAFA, MAFB, and any combinations thereof.

38. The assay of claim 33, wherein the β-cell marker is PDX-1.

39. (canceled)

40. (canceled)

41. (canceled)

42. (canceled)

43. (canceled)

44. (canceled)

45. (canceled)

46. The assay of claim 33, wherein the pancreatic cells are primary pancreatic β-cells.

47. (canceled)

48. The assay of claim 33, wherein the pancreatic cells are derived from de-differentiated cells.

49. (canceled)

50. The assay of claim 33, the assay comprising:
(a) trypsinizing islets of Langerhans into cellular clusters of 1-3 cells;
(b) allowing the cells to recover overnight;
(c) plating the cells from step (b) into the wells of a 96-well plate, wherein the wells are coated with 804G conditioned media and cellular plating density is 60 k cells/well;
(d) allowing the cells to adhere to surface of the wells for at 48 hours;
(e) contacting 1 μM of test compound with the beta-cells for 24 hours;
(f) staining the cells with PDX-1 antibody and Ki-67 and/or PH3 antibody;
(g) assessing beta-cell replication; and
selecting the compound that increases or enhances β-cell replication.
